(19) United States
(12) Patent Application Publication
(10) Pub. No.: US 2010/0166734 A1
(43) Pub. Date: Jul. 1, 2010

(54) ORAL DELIVERY OF POLYPEPTIDES

(76) Inventor: Edward Dolk, Utrecht (NL)

Correspondence Address: WOLF GREENFIELD & SACKS, P.C., 600 ATLANTIC AVENUE, BOSTON, MA, 02210-2206, US

(21) Appl. No.: 12/520,272

(22) PCT Filed: Dec. 20, 2001
(86) PCT No.: PCT/EP07/64344

§ 371 Date: Jan. 1, 2010

(30) Foreign Application Priority Data
Dec. 20, 2006 (US) 60875990

(51) Int. Cl. A61K 38/00; A61K 39/395

(52) U.S. Cl. 424/130.1; 514/2

(57) ABSTRACT

The present invention relates to the oral delivery of therapeutic polypeptides comprising one or more single variable domain(s).

The present invention relates to the oral delivery of polypeptides. More particularly, the present invention relates to the oral delivery of polypeptides comprising a single variable domain such as a Nanobody®, a domain antibody, a single domain antibody, a “dAb” or formatted version thereof, e.g. Polypeptides comprising Nanobodies having multivalent or multimeric binding properties (herein Polypeptides of the Invention, see also further description herein for a more detailed description). The present invention provides compositions suitable for oral delivery of said Polypeptides of the Invention. The invention also relates to methods for the treatment of a subject comprising the delivery of said. Polypeptides of the Invention to said subject by the oral route and to methods-of enhancing bioavailability of such Polypeptides of the Invention when administered orally.

Other aspects, embodiments, advantages and applications of the invention will become clear from the further description herein.

Administration of conventional low molecular weight drugs by non-invasive routes has been a well established practice. Therapeutic peptides and proteins, however, are often unstable, have large molecular weights and are polar in nature. These properties lead to poor permeability through biological membranes. When administered orally, they are susceptible to proteolytic degradation in the gastrointestinal tracts and only pass with difficulty into the body fluids. For this reason, therapeutic peptides and proteins have hitherto been administered mostly by injection, infusion or oral delivery.

However, injection, infusion and oral administration are significantly less convenient than, and involve more patient discomfort than, oral administration. Often this inconvenience or discomfort results in substantial patient non-compliance with the treatment regimen. Thus, there is a need in the art for more effective and reproducible oral administration of polypeptides like e.g. single variable domains and/or construct thereof.

Proteolytic enzymes of both the stomach and intestines may degrade polypeptides, rendering them inactive before they can be absorbed into the bloodstream. Any amount of polypeptides that survives proteolytic degradation by proteases of the stomach (typically having acidic pH optima) is later confronted with proteases of the small intestine and enzymes secreted by the pancreas (typically having neutral to basic pH optima). Specific difficulties arising from the oral administration of a polypeptide involve the relatively large size of the molecule, and the charge distribution it carries. This may make it more difficult for a polypeptide to penetrate the mucus along intestinal walls or to cross the intestinal brush border membrane into the blood.

Oral administration of polypeptides has 2 main challenges that are a) degradation by proteolytic enzymes in the stomach and intestine and b) poor absorption, i.e. poor transport of said polypeptide from the apical to the basolateral side of the intestine and release into the blood. Improving oral effectiveness, i.e. increase of the bioavailability of oral polypeptidic drugs, is a clear unmet medical need and important for several reasons.

First, peptides and proteins are expensive to manufacture either by chemical synthesis or recombinant DNA technologies. Therefore, the more one increases bioavailability, the lesser the amounts that will be required in an oral formulation of a therapeutic drug (economic issue).

Second, the greater the bioavailability of an oral peptide, the less the variability in the dosage absorbed by an individual on a day to day basis (safety issue).

Third, the greater the bioavailability of an oral peptide, the less the concern about breakdown products of the peptide since such breakdown products can act as agonists or antagonists of the receptors where the peptide binds to elicit biological activity (safety issue).

Accordingly delivery of therapeutic polypeptides through the oral route receives great attention; it has not been successful and is considered a big hurdle to biological drugs. The main reasons are intrinsic poor permeability of intestinal wall and fast proteolytic degradation in stomach and gut. There is as of today no oral delivery of larger polypeptides (of 100 amino acids and more) approved for human use and there is no established procedure or know how in the art how to formulate a polypeptide for oral to gut-local or systemic delivery, in particular to systemic delivery.

The present inventors have now found that a certain class of therapeutic polypeptides, i.e. the Polypeptides of the Invention (and further described herein below), generally also including peptides but preferably polypeptides that are larger than 100 amino acids in length, can be delivered into the bloodstream via the oral route. The Polypeptides of the Invention can be conveniently administered to a subject by the oral route by means of a composition comprising said Polypeptides of the Invention with the relevant strategies as disclosed herein. Said Polypeptides of the Invention are characterized and partly shown to be one of the following a) more protease resistant than conventional biologics, e.g. conventional antibodies, b) have typically a higher pH stability or as shown herein (can bind in a pH dependent manner), c) have typically a high temperature stability (i.e. having advantages during processes requiring high T. i.e. in processes of formulation, i.e. compaction and/or granulation), d) have typically a high stability to organic solvents, i.e. may show a superior stability profile to e.g. PLGA solvent exposure (PLGA or poly(lactic-co-glycolic acid) is an Food and Drug Administration (FDA) approved copolymer which is used in a host of therapeutic devices), e) have shown to have long time stability, f) are typically small globular domains (e.g. in a monovalent form are about 10 times smaller than conventional antibodies) allowing for high loading capacity of matrix or implant, and/or g) have typically high solubility allowing for high loading and highly concentrated doses.

The invention provides one or more of the following main strategies to achieve orally administered polypeptide delivery: a) inhibit proteolytic activity that degrades polypeptides in stomach and gut, b) develop protease-resistant polypeptide analogs that retain biological activity, c) stabilize the polypeptide by conjugation to shielding molecules, d) protect the polypeptide from proteolytic degradation by e.g. enteric coating, e) improve passive polypeptide transport (diffusion) through the epithelial membrane of the intestine, f) improve active (e.g. receptor mediated or M-cell mediated) trans-epithelial transport of the polypeptides, and/or g) increase half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life.

In one of the embodiments of the Polypeptides of the Invention, e.g. Nanobodies or dAbs, preferably Nanobodies, systemic and/or local (i.e. topical gut) delivery is provided through oral administration by protecting said polypeptides from proteolytic degradation by e.g. enteric coatings known to the skilled person in the art, e.g. Eudragit L30D-55 (Roehm Pharma Polymers).

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art, e.g. Eudragit L30D-55 (Roehm Pharma Polymers); and b) improving active (e.g. receptor mediated) trans-epithelial transport of said polypeptides, e.g. by pIgR, FcRn, and/or VitB12 receptor mediated trans-epithelial transport, preferably pIgR and/or FcRn, more preferably FcRn mediated trans-epithelial transport.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art, e.g. Eudragit L30D-55 (Roehm Pharma Polymers); and b) improving active (e.g. receptor mediated) trans-epithelial transport of said polypeptides, e.g. by pIgR, FcRn, and/or VitB12 receptor mediated trans-epithelial transport, preferably pIgR and/or FcRn, more preferably FcRn mediated trans-epithelial transport; and c) increasing half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life, e.g. fused Fc fragment, albumin, albumin binder, FcRn binder, and/or serum protein binder. In a preferred embodiment, the unit extending half-life is also able to improve active (e.g. receptor mediated) trans-epithelial transport of said polypeptides, e.g. a FcRn binding unit is able to prolong half/life and improve active receptor mediated trans-epithelial transport in the gut.

In another embodiment of the Polypeptides of the Invention, e.g. Nanobodies or dAbs, preferably Nanobodies, systemic and/or local (i.e. topical gut) delivery is provided through oral administration provided by a) protecting said polypeptides from proteolytic degradation by e.g. enteric coatings known to the skilled person in the art, e.g. Eudragit L30D-55 (Roehm Pharma Polymers); and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport.

In another embodiment of the Polypeptides of the Invention, e.g. Nanobodies or dAbs, preferably Nanobodies, systemic and/or local (i.e. topical gut) delivery is provided through oral administration provided by a) protecting said polypeptides from proteolytic degradation by e.g. enteric coatings known to the skilled person in the art, e.g. Eudragit L30D-55 (Roehm Pharma Polymers); and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport; and c) increasing half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life, e.g. fused Fc fragment, albumin, albumin binder, FcRn binder, and/or serum protein binder. In a preferred embodiment, the unit extending half-life is also able to improve active (e.g. receptor mediated) trans-epithelial transport of said polypeptides, e.g. a FcRn binding unit is able to prolong half/life and improve active receptor mediated trans-epithelial transport in the gut.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art, e.g. Eudragit L30D-55 (Roehm Pharma Polymers); and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport; and [c) inhibit proteolytic activity that degrades polypeptides in stomach and gut by e.g. protease inhibitors such as e.g. organic acids; and/or d) improve passive polypeptide transport (diffusion) through the mucus and epithelial membrane by e.g. permeation enhancer such as acylcarnitine and/or Eligen® carrier technology].

In another embodiment of the Polypeptides of the Invention, e.g. Nanobodies or dAbs, preferably Nanobodies, systemic and/or local (i.e. topical gut) delivery is provided through oral administration provided by a) protecting said polypeptides from proteolytic degradation by e.g. enteric coatings known to the skilled person in the art, e.g. Eudragit L30D-55 (Roehm Pharma Polymers); and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport; and c) increasing half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life, e.g. fused Fc fragment, albumin, albumin binder, FcRn binder, and/or serum protein binder; and [d) inhibit proteolytic activity that degrades polypeptides in stomach and gut by e.g. protease inhibitors such as e.g. organic acids; and/or e) improve passive polypeptide transport (diffusion) through the mucus and epithelial membrane by e.g. permeation enhancer such as acylcarnitine and/or Eligen® carrier technology].

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art, e.g. Eudragit L30D-55 (Roehm Pharma Polymers); and b) improving active (e.g. receptor mediated) trans-epithelial transport of said polypeptides, e.g. by pIgR, FcRn, and/or VitB12 receptor mediated trans-epithelial transport, preferably pIgR and/or FcRn, more preferably FcRn mediated trans-epithelial transport; and [c) inhibit proteolytic activity that degrades polypeptides in stomach and gut by e.g. protease inhibitors such as e.g. organic acids; and/or d) improve passive polypeptide transport (diffusion) through the mucus and epithelial membrane by e.g. permeation enhancer such as acylcarnitine and/or Eligen® carrier technology].

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art, e.g. Eudragit L30D-55 (Roehm Pharma Polymers); and b) providing continuous local (topical in gut) delivery by bacterial system, e.g. lactit acid bacteria.

The present invention, accordingly, relates to a method for the delivery or administration (both terms are used interchangeably throughout the invention) of a Polypeptide of the Invention to the bloodstream and/or other organ and/or tissue (e.g. the kidney, heart, liver, bladder, lung and/or brain) of a subject without being substantially inactivated (i.e. maintaining to a large part its functionality or delivery is such that a safe and efficacious delivery to the target side is provided), comprising the step of administering to said subject by the oral route a composition comprising said Polypeptide of the Invention. The present invention provides a pharmaceutical composition (hereafter referred to as the Pharmaceutical Composition of the Invention) comprising the Polypeptide of the Invention, wherein said polypeptide is designed at least partly in such a way as disclosed herein. The Polypeptide of the Invention has an amino acid sequence that at least comprises one or more single variable domain(s), e.g. a Nanobody, a domain antibody, a single domain antibody or a “dAb”. In a preferred embodiment, the Polypeptide of the Invention has an amino acid sequence essentially consisting of one or more single variable domain(s), e.g. a Nanobody, a domain antibody, a single domain antibody or a “dAb”. In a further preferred embodiment, the Polypeptide of the Invention has an amino acid sequence essentially consisting of one or more single variable domain(s), e.g. a Nanobody (which is also called the Nanobody of the Invention). In a further embodiment, the Nanobody, domain antibody, single domain antibody or “dAb” is derived from a V_{H} or V_{HH}. As described further in the detailed description, the Polypeptide of the Invention comprises a single amino acid chain that can be considered to comprise “framework sequences” or “FR's” and “complementarity determining regions” or “CDR's”.

It is also within the scope of the invention to use parts, fragments, analogs, mutants, variants, alleles and/or derivatives of the Polypeptides of the Invention, and/or to use Polypeptides of the Invention comprising or essentially consisting of the same, as long as these are suitable for the uses envisaged herein. Such parts, fragments, analogs, mutants, variants, alleles, and derivatives will be described in the further description herein.

According to a specific, but non-limiting embodiment, the amino acid sequences of the Nanobodies and/or Polypeptides of the Invention can be “humanized”, “camelized” or modified as further described herein.

Generally, Polypeptides of the Invention that comprise or essentially consist of a single variable domain, e.g. a single Nanobody, domain antibody, single domain antibody or “dAb” will be referred to herein also as “monovalent” polypeptides or as “monovalent constructs”. Polypeptides of the Invention that comprise or essentially consist of two or more single variable domains, e.g. Nanobodies, domain antibodies, single domain antibodies or “dAb's” will be referred to herein also as “multivalent” polypeptides or as “multivalent constructs”, and these may provide certain advantages compared to the corresponding monovalent single variable domains, e.g. Nanobodies, domain antibodies, single domain antibodies or “dAb's”.

According to one specific, but non-limiting embodiment, Polypeptides of the Invention comprise or essentially consist of at least two single variable domains, e.g. Nanobodies, domain antibodies, single domain antibodies or “dAb's”, such as two or three, preferably two, single variable domains, e.g. Nanobodies, domain antibodies, single domain antibodies or “dAb's”. As further described herein, such multivalent constructs can provide certain advantages compared to a polypeptide comprising or essentially consisting of a single variable domain, such as a single Nanobody, domain antibody, single domain antibody or “dAb”, such as a much improved affinity and/or specificity for its antigen. It will be clear for the skilled person how to make such a multivalent constructs from the disclosure herein. According to another specific, but non-limiting embodiment, Polypeptides of the Invention comprise or essentially consist of at least one Nanobody, domain antibody, single domain antibody or “dAb” directed against one epitope, antigen, target, protein or polypeptide and at least one other Nanobody, domain antibody, single domain antibody or “dAb” directed against another epitope of the same target, antigen, target, protein or polypeptide. Such polypeptides are also referred to herein as “multispecific” polypeptides or as ‘multispecific constructs”, and these may provide certain advantages compared to the corresponding monovalent or monospecific Nanobodies, domain antibodies, single domain antibodies or “dAb's”. It will be clear for the skilled person how to make such multispecific constructs from the disclosure herein.

According to yet another specific, but non-limiting embodiment, Polypeptides of the Invention comprise or essentially consist of at least one Nanobody, domain antibody, single domain antibody or “dAb”, optionally one or more further Nanobodies, domain antibodies, single domain antibodies or “dAb's” and at least one other amino acid sequence that adds at least one desired property to the Nanobody, domain antibody, single domain antibody or “dAb” and/or to a resulting fusion protein. Again, such fusion proteins may provide certain advantages compared to the corresponding monovalent Nanobodies, domain antibodies, single domain antibodies or “dAbs”. Some non-limiting examples of such amino acid sequences and of such fusion constructs will become clear from the further description herein. In a specific embodiment, said at least one other amino acid sequence provides an increased half-life to the Polypeptides of the Invention without said other amino acid sequence. In another specific embodiment, said at least one other amino acid sequence, e.g. Fc polypeptide, allows the Polypeptides of the Invention to be directed towards, penetrate and/or cross the mucosal membrane and/or the blood brain barrier.

In the above constructs, the one or more Nanobodies, domain antibodies, single domain antibodies or “dAbs” and/or other amino acid sequences may be directly linked or linked via one or more linker sequences. Some suitable but non-limiting examples of such linkers will become clear from the further description herein. For example, when the one or more groups, residues, moieties or binding units are amino acid sequences, the linkers may also be amino acid sequences, e.g. Ala-Ala-Ala, Gly-Gly-Gly (3-Gly), 9-Gly, or 30-Gly sequence, so that the resulting compound or construct is a fusion (protein) or fusion (polypeptide).

Preferably, Polypeptides of the Invention either comprise one or more Nanobodies, domain antibodies, single domain antibodies or “dAb's”, optionally linked via one or two linkers, or is a multispecific polypeptide, comprising one or more Nanobodies, domain antibodies, single domain antibodies or “dAb's” and at least one Nanobody, domain antibody, single domain antibody or “dAb” that provides an increased half-life following delivery to the subject, particularly providing extended metabolic persistence in an active state within the physiological environment (e.g., in the stomach, at the mucosal surface, in the bloodstream, and/or within another selected physiological compartment, tissue and/or organ such as e.g. the kidney, bladder, lung and/or brain). Examples are a Nanobody, domain antibody, single domain antibody or “dAb” directed against a FcRn, and in particular human FcRn, serum protein, and in particular against a human serum protein, such as against human serum albumin, in which said Nanobodies, domain antibodies, single domain antibodies or “dAb's” again optionally linked via one or more linkers. It will be clear for the skilled person how to make such constructs from the disclosure herein.

In one preferred embodiment of the invention, a Polypeptide of the Invention comprises one or more (such as two or preferably one) Nanobodies, domain antibodies, single domain antibodies or “dAb's” linked (optionally via one or more suitable linker sequences) to one or more (such as two and preferably one) amino acid sequences that allow the resulting Polypeptide of the Invention to be cross the intestine wall of the gut. In particular, said one or more amino acid sequences that allow the resulting Polypeptides of the Invention to cross the intestine wall may be one or more Nanobodies, domain antibodies, single domain antibodies or “dAb's” directed against an M-cell-specific molecule on the epithelial membrane, wherein said Nanobodies, domain antibodies, single domain antibodies or “dAb's” cross the mucosal membrane upon binding to said epithelial transmembrane protein. Mucosa-associated lymphoid tissue in the digestive tracts are covered by a specialized epithelium, the follicle-associated epithelium, which includes M cells, which are specialized for the uptake and transcytosis of macromolecules and microorganisms. Following transcytosis, antigens are released to cells of the immune system in lymphoid aggregates beneath the epithelium where antigen processing and presentation and stimulation of specific B and T lymphocytes are achieved. Circulation of the lymphoid cells enables their homing to their original, and other, mucosal sites where they exert the effector function. Such a response may be dominated by secretory immunoglobulin A release and may include cytotoxic T lymphocyte action. Binding of particles to the apical M cell membrane may be nonspecific or due to specific interaction between molecules such as integrins and lectins. Exploiting the specific binding to M cells is an aim for example to increase the efficiency of uptake of an orally delivered polypeptide by its conjugation to an M-cell-specific molecule. Furthermore, said one or more amino acid sequences that allow the resulting Polypeptides of the Invention to cross the intestine wall may be one or more Nanobodies, domain antibodies, single domain antibodies or “dAb's” directed against the human polymeric immunoglobulin receptor, hpIgR, and/or FcRn, in particular human FcRn.

In another preferred embodiment, a Polypeptide of the Invention comprises one or more (such as two or preferably one) Nanobodies, domain antibodies, single domain antibodies or “dAb's” linked (optionally via one or more suitable linker sequences) to one or more (such as two and preferably one) amino acid sequences that confer an increased half-life in vivo to the resulting Polypeptide of the Invention, in particular, that provides extended metabolic persistence in an active state within the physiological environment (e.g. at the gut epithelial surface, in the bloodstream and/or within another selected physiological compartment, tissue and/or organ such as e.g. the kidney, bladder, lung and/or brain). In particular, said amino acid sequences that confer an increased half-life in vivo to the resulting Polypeptide of the Invention may be one or more (such as two and preferably one) Nanobodies, domain antibodies, single domain antibodies or “dAb's”, and in particular Nanobodies, domain antibodies, single domain antibodies or “dAb's” directed against a human serum protein such as human serum albumin. Examples of suitable Nanobodies against mouse or human serum albumin are described in the applications WO 03/035694, WO 04/041865 and WO 06/122825.

In yet another preferred embodiment, a polypeptide or protein of the invention comprises one or more (such as two or preferably one) Nanobodies, domain antibodies, single domain antibodies or “dAb's”, one or more (such as two and preferably one) amino acid sequences that allow the resulting polypeptide of the invention to be directed towards, penetrate and/or cross the mucosal membrane, and one or more (such as two and preferably one) amino acid sequences that confer an increased half-life in vivo to the resulting polypeptide of the invention, in particular, that provides extended metabolic persistence in an active state within the physiological environment (e.g. at the gut mucosal surface, in the bloodstream and/or within another selected physiological compartment, tissue and/or organ such as e.g. the kidney, bladder, lung and/or brain) (optionally linked via one or more suitable linker sequences). Again, said one or more amino acid sequences that allow the resulting polypeptides of the invention to be directed towards, penetrate and/or cross the mucosal membrane or to cross the blood brain barrier may be one or more (such as two and preferably one) Nanobodies, domain antibodies, single domain antibodies or “dAb's” (as mentioned herein), and said amino acid sequences that confer an increased half-life in vivo to the resulting polypeptide of the invention may be one or more (such as two and preferably one) Nanobodies, domain antibodies, single domain antibodies or “dAb's” (also as mentioned herein).

The compositions of the present invention are formulated for oral administration. Accordingly, in addition to the Polypeptides of the invention, e.g. constructs comprising single variable domains such as e.g. Nanobodies binding to a target molecule and to e.g. FcRn, pIgR or/and VitB12 receptors, the composition of the invention may also comprise a pharmaceutically acceptable oral carrier and, optionally, other therapeutic ingredients or pharmaceutically acceptable additives and/or agents.

Thus, in a further aspect, the present invention relates to a composition that comprises at least a Polypeptide of the Invention (e.g. humanized and e.g. formatted with human FcRn binding unit), optionally an enteric coating (in order to protect said polypeptide from proteolytic), preferably an enteric coating, and at least one further excipient selected from the group consisting of: a) protease inhibitor such as an organic acid); b) proton pump inhibitor such as omeprazole or any other −zoles; c) tonicifiers, d) osmolytes, and/or without being limiting e) surfactants. The use of such additional excipients is well known to those skilled in the art of pharmacology. Some non-limiting examples of such additional excipients are found in e.g. Remington: The Science and Practice of Pharmacy (Remington the Science and Practice of Pharmacy) 21^{st} edition.

Optionally, the composition of the invention also comprises other additives and/or agents. Accordingly, in another embodiment of the invention, a composition is provided comprising a Polypeptide of the Invention (e.g. humanized and e.g. formatted with human FcRn binding unit), optionally an enteric coating (in order to protect said polypeptide from proteolytic), preferably an enteric coating, and one or more pharmaceutically acceptable additives and/or agents. The use of additives such as preservatives, buffering agents, antioxidants, bulking agents and/or viscosity builders are known to those skilled in the art of pharmacology and are also further described e.g. Remington: The Science and Practice of Pharmacy (Remington the Science and Practice of Pharmacy) 21^{st} edition.

In various embodiments, the composition of the invention may comprise a Polypeptide of the Invention (e.g. humanized and e.g. formatted with human FcRn binding unit), optionally an enteric coating (in order to protect said polypeptide from proteolytic), preferably an enteric coating, and optionally, one or more additives and/or agents.

In another embodiment of the invention, the composition may additionally comprise one or more further therapeutic ingredient (or active substances). These combinations of therapeutic ingredients and/or active substances (e.g. also including constructs covalently linking the Polypeptides of the Invention) will also become clear from the further description herein.

The present invention also provides methods for the preparation of a Composition of the Invention. Those methods will also become clear from the further description herein.

The Compositions of the invention are capable of providing a systemic therapeutic or biological activity of the Polypeptide of the Invention, preferably a Polypeptide of the Invention comprising at least a Nanobody and/or dAbs, more preferably a Nanobody, in a subject, following oral administration of said composition comprising said Polypeptide of the Invention to said subject. In an embodiment of the invention, the Polypeptide of the Invention comprising at least a Nanobody and/or dAbs, more preferably a Nanobody, reaches a Cmax in blood of at least 1 ng of Polypeptide comprising at least a Nanobody and/or dAbs, more preferably a Nanobody, per ml of blood. In another embodiment, the Polypeptide of the Invention comprising at least a Nanobody and/or dAbs, more preferably a Nanobody, reaches a Cmax in blood of at least 1 ng of Polypeptide of the Invention comprising at least a Nanobody and/or dAbs, more preferably a Nanobody, per ml of blood following oral administration of a dose of 5 mg/kg body weight of said Polypeptide of the Invention comprising at least a Nanobody and/or dAbs, more preferably a Nanobody. In a further embodiment of the invention, the Polypeptide of the Invention comprising at least a Nanobody and/or dAbs, more preferably a Nanobody, reaches the bloodstream with a Tmax of less than 120 minutes. In another further embodiment, the Polypeptide comprising at least a Nanobody and/or dAbs, more preferably a Nanobody, reaches a Cmax in blood of at least 1 ng of Polypeptide of the Invention comprising at least a Nanobody and/or dAbs, more preferably a Nanobody, per ml of blood within less than 120 minutes following oral administration of the composition comprising said Polypeptide of the Invention comprising at least a Nanobody and/or dAbs, more preferably a Nanobody. In another further embodiment, the Polypeptide of the Invention comprising at least a Nanobody and/or dAbs, more preferably a Nanobody, reaches a Cmax in blood of at least 1 ng of Polypeptide of the Invention comprising at least a Nanobody and/or dAbs, more preferably a Nanobody, per ml of blood within less than 120 minutes following oral administration of a dose of 5 mg/kg body weight of said Polypeptide of the Invention comprising at least a Nanobody and/or dAbs, more preferably a Nanobody. In an embodiment of the invention, the AUC for the Polypeptide of the Invention comprising at least a Nanobody and/or dAbs, more preferably a Nanobody, in blood following oral administration of a composition comprising said Polypeptide of the Invention comprising at least a Nanobody and/or dAbs, more preferably a Nanobody, is at least 500 ng/ml/minute Polypeptide of the Invention comprising at least a Nanobody and/or dAbs, more preferably a Nanobody. In another embodiment of the invention, the AUC for the Polypeptide of the Invention comprising at least a Nanobody and/or dAbs, more preferably a Nanobody, in blood following oral administration of a dose of 5 mg/kg body weight of said Polypeptide of the Invention comprising at least a Nanobody and/or dAbs, more preferably a Nanobody, is at least 500 ng/ml/minute Polypeptide comprising at least a Nanobody and/or dAbs, more preferably a Nanobody. In another further embodiment of the invention, the bioavailability for the Polypeptide of the Invention comprising at least a Nanobody and/or dAbs, more preferably a Nanobody, in blood following oral administration of a composition comprising said Polypeptide comprising at least a Nanobody and/or dAbs, more preferably a Nanobody, is at least 1%, preferably 2%, 3% or 4%, more preferably 5%, most preferred 10%, compared to parenteral administration of said Polypeptide of the Invention comprising at least a Nanobody and/or dAbs, more preferably a Nanobody.

In yet another aspect, the Composition of the Invention is capable of providing a therapeutic or biological activity of the Polypeptide of the Invention comprising at least a Nanobody and/or dAbs, more preferably a Nanobody, in the blood of a subject, following oral administration to said subject of a composition comprising said Polypeptide of the Invention comprising at least a Nanobody and/or dAbs, more preferably a Nanobody. In an embodiment of the invention, the bioavailability for the Polypeptide of the Invention comprising at least a Nanobody and/or dAbs, more preferably a Nanobody in the blood following oral administration of a composition comprising said Polypeptide of the Invention comprising at least a Nanobody and/or dAbs, more preferably a Nanobody is at least 1%, preferably 2%, 3% or 4%, more preferably 5%, most preferred 10%, compared to parenteral administration of said Polypeptide of the Invention comprising at least a Nanobody and/or dAbs, more preferably a Nanobody.

The invention further provides a method for delivering a Polypeptide of the Invention comprising at least a Nanobody and/or dAbs, more preferably a Nanobody, to the bloodstream of a subject without being significantly inactivated or to only such an extent to still fulfill its biological function, said method comprising the step of orally administering a Composition comprising a Polypeptide of the Invention comprising at least a Nanobody and/or dAbs, more preferably a Nanobody, to said subject.

The present invention also provides methods for the prevention and/or treatment of a subject in need of a Polypeptide of the Invention comprising at least a Nanobody and/or dAbs, more preferably a Nanobody, comprising the step of orally administering to said subject a composition as described above and/or below. Further therapeutic applications of the compositions of the invention are described in detail hereafter.

### FIGURES

FIG. 1: hFcRn HC binding assay at different pH for a selection of clones. Negative controls are addition of irrelevant phage selected against a viral antigen and no phage addition.

### DETAILED DESCRIPTION

The above and other aspects, embodiments and advantages of the invention will become clear from the further description herein below.

### DEFINITIONS

a) By the term “Target Molecule” or “Target Molecules” or “target” is meant a protein with a biological function in an organism, preferably animal, more preferably mammal most preferred human, wherein said biological function may be involved in the initiation or progression or maintenance of a disease. Preferably said protein is selected from the group consisting of: human growth hormone(hGH), N-methionyl human growth hormone, bovine growth hormone, parathyroid hormone, thyroxine, insulin A-chain, insulin B-chain, proinsulin, relaxin A-chain, relaxin B-chain, prorelaxin, glycoprotein hormones such as follicle stimulating hormones(FSH), thyroid stimulating hormone(TSH), and leutinizing hormone(LH), glycoprotein hormone receptors, calcitonin, glucagon, factor VIII, an antibody, a Nanobody, a molecule which is well tolerated by mammals in particularly humans and has a long half life when given systemically and/or locally, e.g. poly glycol chains of different size, e.g. PEG-20, PEG-30 or PEG40, lung surfactant, urokinase, streptokinase, human tissue-type plasminogen activator (t-PA), bombesin, factor IX, thrombin, hemopoietic growth factor, tumor necrosis factor-alpha and -beta, enkephalinase, human serum albumin, mullerian-inhibiting substance, mouse gonadotropin-associated peptide, a microbial protein, such as betalactamase, tissue factor protein, inhibin, activin, vascular endothelial growth factor, receptors for hormones or growth factors; integrin, thrombopoietin, protein A or D, rheumatoid factors, nerve growth factors such as NGF-β, platelet-growth factor, transforming growth factors (TGF) such as TGF-alpha and TGF-beta, insulin-like growth factor-I and -II, insulin-like growth factor binding proteins, CD-4, DNase, latency associated peptide, erythropoietin, osteoinductive factors, interferons such as interferon-alpha, -beta, and -gamma, colony stimulating factors (CSFs) such as M-CSF, GF-CSF, and G-CSF, interleukins (ILs) such as IL-1, IL-2, IL-3, IL-4, IL-5, IL-6, van Willebrand factor, superoxide dismutase; decay accelerating factor, viral antigen, HIV envelope proteins such as GP120, GP140, atrial natriuretic peptides A, B or C, immunoglobulins, and fragments or variants of any of the above-listed proteins. More preferably, said Target Molecule is a multimeric protein and even more preferred is a multimeric protein which subunits are selected from the group consisting of: van Willebrand Factor (vWF), IL-6, tumor necrosis factor-alpha and -beta and many others. A multimeric protein is a protein which is associated (typically by non-covalent interactions) in biological organism such as humans with others as subunits in a multimeric structure and typically only in the multimeric format is able to unfold its biological function. This is also called the quaternary structure of the protein. This association can also be stabilized by disulfide bonds and by noncovalent interactions with reacting substrates or cofactors.
b) The single variable domains that are present in the constructs of the invention may be any variable domain that forms a single antigen binding unit. Generally, such single variable domains will be amino acid sequences that essentially consist of 4 framework regions (FR1 to FR4 respectively) and 3 complementarity determining regions (CDR1 to CDR3 respectively); or any suitable fragment of such an amino acid sequence (which will then usually contain at least some of the amino acid residues that form at least one of the CDR's, as further described herein). Such single variable domains and fragments are most preferably such that they comprise an immunoglobulin fold or are capable for forming, under suitable conditions, an immunoglobulin fold. As such, the single variable domain may for example comprise a light chain variable domain sequence (e.g. a V_{L}-sequence) or a suitable fragment thereof; or a heavy chain variable domain sequence (e.g. a V_{H}-sequence or V_{HH} sequence) or a suitable fragment thereof; as long as it is capable of forming a single antigen binding unit (i.e. a functional antigen binding unit that essentially consists of the single variable domain, such that the single antigen binding domain does not need to interact with another variable domain to form a functional antigen binding unit, as is for example the case for the variable domains that are present in for example conventional antibodies and ScFv fragments that need to interact with another variable domain—e.g. through a V_{H}/V_{L} interaction—to form a functional antigen binding domain).
   For example, the single variable domain may be a domain antibody (or an amino acid sequence that is suitable for use as a domain antibody), a single domain antibody (or an amino acid sequence that is suitable for use as a single domain antibody), a “dAb” or dAb (or an amino acid sequence that is suitable for use as a dAb) or a Nanobody™ (as defined herein, and including but not limited to a V_{HH} sequence); other single variable domains, or any suitable fragment of any one thereof. For a general description of (single) domain antibodies, reference is also made to the prior art cited above, as well as to EP 0 368 684. For the term “dAb's”, reference is for example made to Ward et al. (Nature 1989 Oct. 12; 341 (6242): 544-6), to Holt et al., Trends Biotechnol., 2003, 21(11):484-490; as well as to for example WO 04/068820, WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called “IgNAR domains”, see for example WO 05/18629).
   In particular, the amino acid sequence of the invention may be a Nanobody™ or a suitable fragment thereof. [Note: Nanobody™, Nanobodies™ and Nanoclone™ are trademarks of Ablynx N.V.] For a further description of V_{HH}'s and Nanobodies, reference is made to the review article by Muyldermans in Reviews in Molecular Biotechnology 74 (2001), 277-302; as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. Reference is also made to the further prior art mentioned in these applications, and in particular to the list of references mentioned on pages 41-43 of the International application WO 06/040153, which list and references are incorporated herein by reference. As described in these references, Nanobodies (in particular V_{HH} sequences and partially humanized Nanobodies) can in particular be characterized by the presence of one or more “Hallmark residues” in one or more of the framework sequences. A further description of the Nanobodies, including humanization and/or camelization of Nanobodies, as well as other modifications, parts or fragments, derivatives or “Nanobody fusions”, multivalent constructs (including some non-limiting examples of linker sequences) and different modifications to increase the half-life of the Nanobodies and their preparations can be found e.g. in WO07/104,529.
c) By “high affinity” as used herein is meant a dissociation constant for a monovalent binding Nanobody of (Kd) of <100 nM and preferably 10 nM and more preferably 1 nM and even more preferably 100 pM and most preferred 10 pM under physiological conditions and measured by standard procedures in the art.
d) By “high avidity” as used herein is meant a dissociation constant for a bi- or multivalent binding Nanobody of (Kd) of <100 nM and preferably 10 nM and more preferably 1 nM and even more preferably 100 pM and most preferred 10 pM under physiological conditions and measured by standard procedures in the art.
e) By “rigid secondary structure” as used herein is meant any polypeptide segment exhibiting a regular repeated structure such as is found in; α-helices, 310 helices, π-helices, parallel and antiparallel β-sheets, and reverse turns. Certain “non-ordered” structures that lack recognizable geometric order are also included in the definition of rigid secondary structure provided they form a domain or “patch” of amino acid residues capable of interaction with a target and that the overall shape of the structure is not destroyed by replacement of an amino acid within the structure. It is believed that some non-ordered structures are combinations of reverse turns. The geometry of these rigid secondary structures is well defined by φ and .psi. torsional angles about the α-carbons of the peptide “backbone”. The requirement that the secondary structure be exposed to the surface of the polypeptide is to provide a domain or “patch” of amino acid residues that can be exposed to and bind with a target molecule. It is primarily these amino acid residues that are replaced by mutagenesis that form the “library” of structurally related (mutant) binding polypeptides that are displayed on the surface of the phage and from which novel polypeptide ligands are selected. Mutagenesis or replacement of amino acid residues directed toward the interior of the polypeptide is generally avoided so that the overall structure of the rigid secondary structure is preserved. Some replacement of amino acids on the interior region of the rigid secondary structures, especially with hydrophobic amino acid residues, may be tolerated since these conservative substitutions are unlikely to distort the overall structure of the polypeptide.
f) By “leader sequence” as used herein is meant a particular section of messenger RNA (mRNA) and the DNA that codes for it. It starts at the +1 position (where transcription begins) and ends just before the start codon (usually AUG) of the coding region. It usually contains a ribosome binding site (RBS), in bacteria also known as the Shine-Delgarno sequence (AGGAGGU). The 5′ UTR may be a hundred or more nucleotides long, and the 3′ UTR may be even longer (up to several kilobases in length) (Molecular Cell Biology, 5th edition, Lodish et al. p113, chapter 4.2). Unless indicated or defined otherwise, all terms used have their usual meaning in the art, which will be clear to the skilled person. Reference is for example made to the standard handbooks, such as Sambrook et al. “Molecular Cloning: A Laboratory Manual” (2nd.Ed.), Vols. 1-3, Cold Spring Harbor Laboratory Press (1989); F. Ausubel et al, eds., “Current protocols in molecular biology”, Green. Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, New York (2005), as well as to the general background art cited herein;
g) Unless indicated otherwise, the term “immunoglobulin sequence”—whether used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L}, domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acids or nucleotide sequences encoding the same, unless the context requires a more limited interpretation. Also, the term “nucleotide sequence” as used herein also encompasses a nucleic acid molecule with said nucleotide sequence, so that the terms “nucleotide sequence” and “nucleic acid” should be considered equivalent and are used interchangeably herein;
h) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2(1): 49-57; Irving et al., J. Immunol. Methods, 2001, 248(1-2), 31-45; Schmitz et al., Placenta, 2000, 21 Suppl. A, S106-12, Gonzales et al., Tumour Biol., 2005, 26(1), 31-43, which describe techniques for protein engineering, such as affinity maturation and other techniques for improving the specificity and other desired properties of proteins such as immunoglobulins.
i) Amino acid residues will be indicated according to the standard three-letter or one-letter amino acid code, as mentioned in Table A-2;

TABLE A-2
one-letter and three-letter amino acid code

| | | | |
|---|---|---|---|
| Nonpolar, | Alanine | Ala | A |
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residue is greatly dependant upon even small shifts in pH, but a His residu can generally be considered essentially uncharged at a pH of about 6.5.

j) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position). Alternatively, the degree of sequence identity between two or more nucleotide sequences may be calculated using a known computer algorithm for sequence alignment such as NCBI Blast v2.0, using standard settings. Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A. Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
k) For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence. Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.
   Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J Molec. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
l) Amino acid sequences and nucleic acid sequences are said to be “exactly the same” if they have 100% sequence identity (as defined herein) over their entire length;
m) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
n) When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the firstmentioned nucleotide sequence or amino acid sequence, respectively, but more usually this generally means that the firstmentioned nucleotide sequence or amino acid sequence comprises within its sequence a stretch of nucleotides or amino acid residues, respectively, that has the same nucleotide sequence or amino acid sequence, respectively, as the latter sequence, irrespective of how the firstmentioned sequence has actually been generated or obtained (which may for example be by any suitable method described herein). By means of a non-limiting example, when a Nanobody of the invention is said to comprise a CDR sequence, this may mean that said CDR sequence has been incorporated into the Nanobody of the invention, but more usually this generally means that the Nanobody of the invention contains within its sequence a stretch of amino acid residues with the same amino acid sequence as said CDR sequence, irrespective of how said Nanobody of the invention has been generated or obtained. It should also be noted that when the latter amino acid sequence has a specific biological or structural function, it preferably has essentially the same, a similar or an equivalent biological or structural function in the firstmentioned amino acid sequence (in other words, the firstmentioned amino acid sequence is preferably such that the latter sequence is capable of performing essentially the same, a similar or an equivalent biological or structural function). For example, when a Nanobody of the invention is said to comprise a CDR sequence or framework sequence, respectively, the CDR sequence and framework are preferably capable, in said Nanobody, of functioning as a CDR sequence or framework sequence, respectively. Also, when a nucleotide sequence is said to comprise another nucleotide sequence, the firstmentioned nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the firstmentioned, larger nucleotide sequence).
o) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
p) The term “domain” as used herein generally refers to a globular region of an amino acid sequence (such as an antibody chain, and in particular to a globular region of a heavy chain antibody), or to a polypeptide that essentially consists of such a globular region. Usually, such a domain will comprise peptide loops (for example 3 or 4 peptide loops) stabilized, for example, as a sheet or by disulfide bonds. The term “binding domain” refers to such a domain that is directed against an antigenic determinant (as defined herein);
q) The term “antigenic determinant” refers to the epitope on the antigen recognized by the antigen-binding molecule (such as a Nanobody or a polypeptide of the invention) and more in particular by the antigen-binding site of said molecule. The terms “antigenic determinant” and “epitope” may also be used interchangeably herein.
r) An amino acid sequence (such as a Nanobody, an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
s) The term “specificity” refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the amino acid sequences, Nanobodies and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e,g, of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].
   The affinity denotes the strength or stability of a molecular interaction. The affinity is commonly given as by the K_{D}, or dissociation constant, which has units of mol/liter (or M). The affinity can also be expressed as an association constant, K_{A}, which equals 1/K_{D} and has units of (mol/liter)⁻¹ (or M⁻¹). In the present specification, the stability of the interaction between two molecules (such as an amino acid sequence, Nanobody or polypeptide of the invention and its intended target) will mainly be expressed in terms of the K_{D} value of their interaction; it being clear to the skilled person that in view of the relation K_{A}=1/K_{D}, specifying the strength of molecular interaction by its K_{D} value can also be used to calculate the corresponding K_{A} value. The K_{D}-value characterizes the strength of a molecular interaction also in a thermodynamic sense as it is related to the free energy (DG) of binding by the well known relation DG=RT.1n(K_{D}) (equivalently DG=−RT.1n(K_{A})), where R equals the gas constant, T equals the absolute temperature and ln denotes the natural logarithm.
   The K_{D} for biological interactions which are considered meaningful (e.g. specific) are typically in the range of 10⁻¹⁰M (0.1 nM) to 10⁻⁵M (10000 nM). The stronger an interaction is, the lower is its K_{D}. The K_{D} can also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹. The on-rate may vary between 10² M⁻¹S⁻¹ to about 10⁷ M⁻¹s⁻¹, approaching the diffusion-limited association rate constant for bimolecular interactions. The off-rate is related to the half-life of a given molecular interaction by the relation t_{1/2}=1n(2)/k_{off}. The off-rate may vary between 10⁻⁶ s⁻¹ (near irreversible complex with a t_{1/2} of multiple days) to 1 s⁻¹ (t_{1/2}=0.69 s).
   The affinity of a molecular interaction between two molecules can be measured via different techniques known per se, such as the well known surface plasmon resonance (SPR) biosensor technique (see for example Ober et al., Intern. Immunology, 13, 1551-1559, 2001) where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIACORE instruments. It will also be clear to the skilled person that the measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule. In such situation the measured affinity may be affected by the avidity of the interaction by the two molecules.
   Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artefacts relating to adsorption of one of the molecules on a support such as plastic.
   However, the accurate measurement of K_{D} may be quite labor-intensive and as consequence, often apparent K_{D} values are determined to assess the binding strength of two molecules. It should be noted that as long all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance.
   Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labelled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of A is varied for a given concentration or amount of B. As a result an IC₅₀ value is obtained corresponding to the concentration of A at which the signal measured for C in absence of A is halved. Provided K_{D ref}, the K_{D} of the reference molecule, is known, as well as the total concentration c_{ref} of the reference molecule, the apparent K_{D} for the interaction A-B can be obtained from following formula: K_{D}=IC₅₀/(1+c_{ref}/K_{D ref}). Note that if c_{ref}<<K_{D ref}, K_{D}≈IC₅₀. Provided the measurement of the IC₅₀ is performed in a consistent way (e.g. keeping c_{ref} fixed) for the binders that are compared, the strength or stability of a molecular interaction can be assessed by the IC₅₀ and this measurement is judged as equivalent to K_{D} or to apparent K_{D} throughout this text.
t) The half-life of an amino acid sequence, compound or polypeptide of the invention can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally involve the steps of suitably administering to a warm-blooded animal (i.e. to a human or to another suitable mammal, such as a mouse, rabbit, rat, pig, dog or a primate, for example monkeys from the genus Macaca (such as, and in particular, cynomologus monkeys (Macaca fascicularis) and/or rhesus monkeys (Macaca mulatta)) and baboon (Papio ursinus)) a suitable dose of the amino acid sequence, compound or polypeptide of the invention; collecting blood samples or other samples from said animal; determining the level or concentration of the amino acid sequence, compound or polypeptide of the invention in said blood sample; and calculating, from (a plot of) the data thus obtained, the time until the level or concentration of the amino acid sequence, compound or polypeptide of the invention has been reduced by 50% compared to the initial level upon dosing. Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982).
   As will also be clear to the skilled person (see for example pages 6 and 7 of WO 04/003019 and in the further references cited therein), the half-life can be expressed using parameters such as the t½-alpha, t½-beta and the area under the curve (AUC). In the present specification, an “increase in half-life” refers to an increase in any one of these parameters, such as any two of these parameters, or essentially all three these parameters. As used herein “increase in half-life” or “increased half-life” in particular refers to an increase in the t½-beta, either with or without an increase in the t½-alpha and/or the AUC or both.
u) In the context of the present invention, “modulating” or “to modulate” generally means either reducing or inhibiting the activity of, or alternatively increasing the activity of, a target or antigen, as measured using a suitable in vitro, cellular or in vivo assay. In particular, “modulating” or “to modulate” may mean either reducing or inhibiting the activity of, or alternatively increasing a (relevant or intended) biological activity of, a target or antigen, as measured using a suitable in vitro, cellular or in vivo assay (which will usually depend on the target or antigen involved), by at least 1%, preferably at least 5%, such as at least 10% or at least 25%, for example by at least 50%, at least 60%, at least 70%, at least 80%, or 90% or more, compared to activity of the target or antigen in the same assay under the same conditions but without the presence of the construct of the invention. As will be clear to the skilled person, “modulating” may also involve effecting a change (which may either be an increase or a decrease) in affinity, avidity, specificity and/or selectivity of a target or antigen for one or more of its ligands, binding partners, partners for association into a homomultimeric or heteromultimeric form, or substrates; and/or effecting a change (which may either be an increase or a decrease) in the sensitivity of the target or antigen for one or more conditions in the medium or surroundings in which the target or antigen is present (such as pH, ion strength, the presence of co-factors, etc.), compared to the same conditions but without the presence of the construct of the invention. As will be clear to the skilled person, this may again be determined in any suitable manner and/or using any suitable assay known per se, depending on the target or antigen involved.
   “Modulating” may also mean effecting a change (i.e. an activity as an agonist, as an antagonist or as a reverse agonist, respectively, depending on the target or antigen and the desired biological or physiological effect) with respect to one or more biological or physiological mechanisms, effects, responses, functions, pathways or activities in which the target or antigen (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, depending on the target or antigen involved. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the construct of the invention. Modulating may for example also involve allosteric modulation of the target or antigen; and/or reducing or inhibiting the binding of the target or antigen to one of its substrates or ligands and/or competing with a natural ligand, substrate for binding to the target or antigen. Modulating may also involve activating the target or antigen or the mechanism or pathway in which it is involved. Modulating may for example also involve effecting a change in respect of the folding or confirmation of the target or antigen, or in respect of the ability of the target or antigen to fold, to change its confirmation (for example, upon binding of a ligand), to associate with other (sub)units, or to disassociate. Modulating may for example also involve effecting a change in the ability of the target or antigen to transport other compounds or to serve as a channel for other compounds (such as ions). Modulating may be reversible or irreversible, but for pharmaceutical and pharmacological purposes will usually be in a reversible mariner.
v) In respect of a target or antigen, the term “interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen. More generally, an “interaction site” can be any site, epitope, antigenic determinant, part, domain or stretch of amino acid residues on the target or antigen to which an amino acid sequence or polypeptide of the invention can bind such that the target or antigen (and/or any pathway, interaction, signalling, biological mechanism or biological effect in which the target or antigen is involved) is modulated (as defined herein).
w) An amino acid sequence or polypeptide is said to be “specific for” a first target or antigen compared to a second target or antigen when is binds to the first antigen with an affinity (as described above, and suitably expressed as a K_{D} value, K_{A} value, K_{off} rate and/or Kₒₙ rate) that is at least 10 times, such as at least 100 times, and preferably at least 1000 times, and up to 10.000 times or more better than the affinity with which said amino acid sequence or polypeptide binds to the second target or polypeptide. For example, the first antigen may bind to the target or antigen with a K_{D} value that is at least 10 times less, such as at least 100 times less, and preferably at least 1000 times less, such as 10.000 times less or even less than that, than the K_{D} with which said amino acid sequence or polypeptide binds to the second target or polypeptide. Preferably, when an amino acid sequence or polypeptide is “specific for” a first target or antigen compared to a second target or antigen, it is directed against (as defined herein) said first target or antigen, but not directed against said second target or antigen.
x) The terms “cross-block”, “cross-blocked” and “cross-blocking” are used interchangeably herein to mean the ability of an amino acid sequence or other binding agents (such as a polypeptide of the invention) to interfere with the binding of other amino acid sequences or binding agents of the invention to a given target. The extend to which an amino acid sequence or other binding agents of the invention is able to interfere with the binding of another to [target], and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between amino acid sequence or another binding agents in terms of their binding to the target.
   The following generally describes a suitable Biacore assay for determining whether an amino acid sequence or other binding agent cross-blocks or is capable of cross-blocking according to the invention. It will be appreciated that the assay can be used with any of the amino acid sequence or other binding agents described herein. The Biacore machine (for example the Biacore 3000) is operated in line with the manufacturer's recommendations. Thus in one cross-blocking assay, the target protein is coupled to a CM5 Biacore chip using standard amine coupling chemistry to generate a surface that is coated with, the target. Typically 200-800 resonance units of the target would be coupled to the chip (an amount that gives easily measurable levels of binding but that is readily saturable by the concentrations of test reagent being used). Two test amino acid sequences (termed A* and B*) to be assessed for their ability to cross-block each other are mixed at a one to one molar ratio of binding sites in a suitable buffer to create the test mixture. When calculating the concentrations on a binding site basis the molecular weight of an amino acid sequence is assumed to be the total molecular weight of the amino acid sequence divided by the number of target binding sites on that amino acid sequence. The concentration of each amino acid sequence in the test mix should be high enough to readily saturate the binding sites for that amino acid sequence on the target molecules captured on the Biacore chip. The amino acid sequences in the mixture are at the same molar concentration (on a binding basis) and that concentration would typically be between 1.00 and 1.5 micromolar (on a binding site basis). Separate solutions containing A* alone and B* alone are also prepared. A* and B* in these solutions should be in the same buffer and at the same concentration as in the test mix. The test mixture is passed over the target-coated Biacore chip and the total amount of binding recorded. The chip is then treated in such a way as to remove the bound amino acid sequences without damaging the chip-bound target. Typically this is done by treating the chip with 30 mM HCl for 60 seconds. The solution of A* alone is then passed over the target-coated surface and the amount of binding recorded. The chip is again treated to remove all of the bound amino acid sequences without damaging the chip-bound target. The solution of B* alone is then passed over the target-coated surface and the amount of binding recorded. The maximum theoretical binding of the mixture of A* and B* is next calculated, and is the sum of the binding of each amino acid sequence when passed over the target surface alone. If the actual recorded binding of the mixture is less than this theoretical maximum then the two amino acid sequences are cross-blocking each other. Thus, in general, a cross-blocking amino acid sequence or other binding agent according to the invention is one which will bind to the target in the above Biacore cross-blocking assay such that during the assay and in the presence of a second amino acid sequence or other binding agent of the invention the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two amino acid sequences or binding agents in combination. The Biacore assay described above is a primary assay used to determine if amino acid sequences or other binding agents cross-block each other according to the invention. On rare occasions particular amino acid sequences or other binding agents may not bind to target coupled via amine chemistry to a CM5 Biacore chip (this usually occurs when the relevant binding site on target is masked or destroyed by the coupling to the chip). In such cases cross-blocking can be determined using a tagged version of the target, for example a N-terminal His-tagged version (R & D Systems, Minneapolis. Minn., USA; 2005 cat #1406-ST-025). In this particular format, an anti-His amino acid sequence would be coupled to the Biacore chip and then the His-tagged target would be passed over the surface of the chip and captured by the anti-His amino acid sequence. The cross blocking analysis would be carried out essentially as described above, except that after each chip regeneration cycle, new His-tagged target would be loaded back onto the anti-His amino acid sequence coated surface. In addition to the example given using N-terminal His-tagged [target], C-terminal His-tagged target could alternatively be used. Furthermore, various other tags and tag binding protein combinations that are known in the art could be used for such a cross-blocking analysis (e.g. HA tag with anti-HA antibodies; FLAG tag with anti-FLAG antibodies; biotin tag with streptavidin).
   The following generally describes an ELISA assay for determining whether an amino acid sequence or other binding agent directed against a target cross-blocks or is capable of cross-blocking as defined herein. It will be appreciated that the assay can be used with any of the amino acid sequences (or other binding agents such as polypeptides of the invention) described herein. The general principal of the assay is to have an amino acid sequence or binding agent that is directed against the target coated onto the wells of an ELISA plate. An excess amount of a second, potentially cross-blocking, anti-target amino acid sequence is added in solution (i.e. not bound to the ELISA plate). A limited amount of the target is then added to the wells. The coated amino acid sequence and the amino acid sequence in solution compete for binding of the limited number of target molecules. The plate is washed to remove excess target that has not been bound by the coated amino acid sequence and to also remove the second, solution phase amino acid sequence as well as any complexes formed between the second, solution phase amino acid sequence and target. The amount of bound target is then measured using a reagent that is appropriate to detect the target. An amino acid sequence in solution that is able to cross-block the coated amino acid sequence will be able to cause a decrease in the number of target molecules that the coated amino acid sequence can bind relative to the number of target molecules that the coated amino acid sequence can bind in the absence of the second, solution phase, amino acid sequence. In the instance where the first amino acid sequence, e.g. an Ab-X, is chosen to be the immobilized amino acid sequence, it is coated onto the wells of the ELISA plate, after which the plates are blocked with a suitable blocking solution to minimize non-specific binding of reagents that are subsequently added. An excess amount of the second amino acid sequence, i.e. Ab-Y, is then added to the ELISA plate such that the moles of Ab-Y [target] binding sites per well are at least 10 fold higher than the moles of Ab-X [target] binding sites that were used, per well, during the coating of the ELISA plate. [target] is then added such that the moles of [target] added per well are at least 25-fold lower than the moles of Ab-X [target] binding sites that were used for coating each well. Following a suitable incubation period the ELISA plate is washed and a reagent for detecting the target is added to measure the amount of target specifically bound by the coated anti-[target] amino acid sequence (in this case Ab-X). The background signal for the assay is defined as the signal obtained in wells with the coated amino acid sequence (in this case Ab-X), second solution phase amino acid sequence (in this case Ab-Y), [target] buffer only (i.e. no target) and target detection reagents. The positive control signal for the assay is defined as the signal obtained in wells with the coated amino acid sequence (in this case Ab-X), second solution phase amino acid sequence buffer only (i.e. no second solution phase amino acid sequence), target and target detection reagents. The ELISA assay may be run in such a manner so as to have the positive control signal be at least 6 times the background signal. To avoid any artefacts (e.g. significantly different affinities between Ab-X and Ab-Y for [target]) resulting from the choice of which amino acid sequence to use as the coating amino acid sequence and which to use as the second (competitor) amino acid sequence, the cross-blocking assay may to be run in two formats: 1) format 1 is where Ab-X is the amino acid sequence that is coated onto the ELISA plate and Ab-Y is the competitor amino acid sequence that is in solution and 2) format 2 is where Ab-Y is the amino acid sequence that is coated onto the ELISA plate and Ab-X is the competitor amino acid sequence that is in solution. Ab-X and Ab-Y are defined as cross-blocking if, either in format 1 or in format 2, the solution phase anti-target amino acid sequence is able to cause a reduction of between 60% and 100%, specifically between 70% and 100%, and more specifically between 80% and 100%, of the target detection signal {i.e. the amount of target bound by the coated amino acid sequence) as compared to the target detection signal obtained in the absence of the solution phase anti-target amino acid sequence (i.e. the positive control wells).
y) As further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
z) The amino acid residues of a Nanobody are numbered according to the general numbering for V_{H} domains given by Kabat et al. (“Sequence of proteins of immunological interest”, US Public Health. Services, NIH Bethesda, Md., Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication); or referred to herein. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.].
   Alternative methods for numbering the amino acid residues of V_{H} domains, which methods can also be applied in an analogous manner to V_{HH} domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise; and
aa) The Figures, Sequence Listing and the Experimental Part/Examples are only given to further illustrate the invention and should not be interpreted or construed as limiting the scope of the invention and/or of the appended claims in any way, unless explicitly indicated otherwise herein.

Without being limited thereto, Nanobodies, (single) domain antibodies or “dAb's” can be derived from the variable region of a 4-chain antibody as well as from the variable region of a heavy chain antibody. In accordance with the terminology used in the references below, the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

Thus—without being limited thereto—the polypeptide or protein of the invention has an amino acid sequence that comprises or essentially consists of four framework regions (FR1 to FR4, respectively) and three complementarity determining regions (CDR1 to CDR3, respectively). Such an amino acid sequence preferably contains between 80 and 200 amino acid residues, such as between 90 and 150 amino acid residues, such as about 100-130 amino acid residues (although suitable fragments of such an amino acid sequence—i.e. essentially as described herein for the Nanobodies of the invention or equivalent thereto—may also be used), and is preferably such that it forms an immunoglobulin fold or such that, under suitable conditions, it is capable of forming an immunoglobulin fold (i.e. by suitable folding). The amino acid sequence is preferably chosen from Nanobodies, domain antibodies, single domain antibodies or “dAb's”, and is most preferably a Nanobody as defined herein. The CDR's may be any suitable CDR's that provide the desired property to the polypeptide or protein.

The invention provides one or more of the following main strategies to achieve orally administered polypeptide delivery: a) inhibition of proteolytic activity that degrades polypeptides in stomach and gut, b) developing of protease-resistant polypeptide analogs that retain biological activity, c) stabilizing the polypeptide by conjugation to shielding molecules, d) protecting the polypeptide from proteolytic degradation by e.g. enteric coating, e) improving active (e.g. receptor mediated or M-cell mediated) trans-epithelial transport of the polypeptides, f) increasing half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life, and/or without being limited to g) improving passive polypeptide transport (diffusion) through the epithelial membrane of the intestine.

a) Inhibition of Proteolytic Activity that Degrades Polypeptides in Stomach and Gut

The Composition of the Invention may comprise agents that inhibit the proteases (i.e. protease inhibitors) present mainly in the stomach but also to a lesser extend in the gut. Such agents are generally known to the skilled person in the art and may be found in e.g. Remington, supra. An example of protease inhibitor is an organic acid such as citric or acetic acid. Protease inhibitors are readily available for the skilled person in the art.

b) Development of Protease-Resistant Polypeptide Analogs that Retain Biological Activity

Hermsen et al. (see Harmsen M M, van Solt C B, van Zijderveld-van Bemmel A M, Niewold T A, van Zijderveld F G. Selection and optimization of proteolytically stable llama single-domain antibody fragments for oral immunotherapy. Appl Microbiol Biotechnol. 2006 Feb. 1; 1-8) showed that stringent selection for proteolytic stability resulted in seven Nanobodies (or VHHs) with 7- to 138-fold increased stability after in vitro incubation in gastric fluid. By DNA shuffling they further obtained four clones with a further 1.5- to 3-fold increased in vitro stability. These Nanobodies or VHHs differed by at most ten amino acid residues from each other and were scattered over the VHH sequence and did not overlap with predicted protease cleavage sites. The most stable clone retained 41% activity after incubation in gastric fluid and 90% in jejunal fluid. Similarly, the invention provides pharmaceutical compositions comprising proteolytically stable single variable domains, e.g. Nanobodies or VHHs, wherein said proteolytically stable Nanobodies can be formatted into bi- and/or multivalent (and multimeric) constructs, e.g. into the constructs, polypeptides of the invention.

### c) Stabilization of the Polypeptide by Conjugation to Shielding Molecules

It is a further embodiment of the invention to provide Polypeptides of the Invention which are conjugated to proteolytically “shielding” molecules such as e.g. pegylated polypeptides comprising single variable domains such as e.g. Nanobodies and/or dAbs. As mentioned herein, the single variable domains, e.g. Nanobodies and/or dAbs and constructs described herein may be pegylated, or contain one or more (additional) amino acid residues that allow for pegylation and/or facilitate pegylation. Two preferred, but non-limiting examples of such polypeptides are TNF55 and TNF56 as described in WO/2006/122786, which both contain an additional cysteine residue for easy attachment of a PEG-group.

d) Protection of the Polypeptide from Proteolytic Degradation by e.g. Enteric Coating

Any enteric coating that protects the peptide from stomach proteases and which releases active components of the invention in the intestine is suitable. The enteric coating functions by providing a coating that does not dissolve in low pH environments, such as the stomach. Many enteric coatings are known in the art, and are useful in accordance with the invention. Examples include cellulose acetate phthalate, hydroxypropylmethylethylcellulose succinate, hydroxypropylmethylcellulose phthalate, polyvinyl acetate phthalate, and methacrylic acid-methyl methacrylate copolymer. It is very desirable that all of the active components be released from the dosage form, and solubilized in the intestinal environment as simultaneously as possible. It is preferred that the dosage form release the active components in the small intestine.

e) Improvement of Active (e.g. Receptor Mediated or M-Cell Mediated) Trans-Epithelial Transport of the Polypeptides

It is also known that Fc receptors are involved in transcytosis recycling of proteins and other (biological) molecules. For example, pIgR, FcRn, and Vit B12 receptor is known to be involved in transcytosis through biological membranes such as epithelial layers, e.g. in adult human gut, and FcRn is known to be involved in the recycling of albumin and IgG (see for example Chaudhury et al., The Journal of Experimental Medicine, vol. 3, no. 197, 315-322 (2003)). Thus, the invention provides building blocks, i.e. single variable domains such as Nanobodies and/or dAbs binding to pIgR, FcRn and/or the Vit B 12 receptor. Furthermore, the building block may also be the natural ligand or fragment of ligand, i.e. human Fc part. It is an embodiment of the invention to provide pharmaceutical compositions comprising the Polypeptides or Constructs of the Invention, wherein said polypeptides comprise a) at least a single, preferably a bivalent, more preferably a bivalent agonistic, variable domain, e.g. a Nanobody, against a Target Molecule, e.g. human growth hormone (hGH) and/or erythropoietin (EPO), and b) epithelial receptor binding single variable domain (e.g. FcRn, Vit B12 or pIgR, preferably FcRn or pIgR, more preferably FcRn, binding Nanobody). Another embodiment of the present invention is a method for selecting Nanobodies, domain antibodies, single domain antibodies or dAbs directed against an epithelial trans-membrane protein, wherein said Nanobody, domain antibody, single domain antibody or dAb crosses the gut membrane upon binding to said epithelial trans-membrane protein. Said method comprises panning epithelial trans-membrane protein-displaying membranes with a phage library (naïve or immune) of Nanobodies, domain antibodies, single domain antibodies or dAbs, and selecting for membrane crossing Nanobodies, domain antibodies, single domain antibodies or dAbs by recovering the transported phage from the membrane. The invention includes a selection method which uses cell lines that over-expresses an epithelial trans-membrane protein or cell lines transfected with an epithelial trans-membrane protein gene to allow the easy selection of phage Nanobodies, domain antibodies, single domain antibodies or dAbs binding to the epithelial trans-membrane protein. This avoids the need for protein expression and purification, speeding up significantly the generation of membrane crossing Nanobodies, domain antibodies, single domain antibodies or dAbs.

In another embodiment, the invention includes a selection method using cells to allow the selection of phage single variable domains, Nanobodies, domain antibodies, single domain antibodies or dAbs that show receptor mediated internalization. Said method comprises adding the phage Nanobodies, domain antibodies, single domain antibodies or dAbs to the cells and recovering the phage Nanobodies, domain antibodies, single domain antibodies or dAbs from the cells that have undergone internalization. In yet another embodiment, the invention includes a selection method using cells seeded on a filter or in a Transwell system or Boyden chamber to allow the selection of phage Nanobodies, domain antibodies, single domain antibodies or dAbs that transcytose through the cell monolayer. Said method comprises adding the phage Nanobodies, domain antibodies, single domain antibodies or dAbs to compartment 1, allow the phage Nanobodies, domain antibodies, single domain antibodies or dAbs to migrate across the cell monolayer and harvest the phage Nanobodies that migrate in compartment 2. Alternatively, the Polypeptides of the Invention comprising e.g. at least a Nanobody or a dAb against a Target Molecule, may also be suitably formulated per se for oral delivery e.g. in the form of a powder (such as a freeze-dried or micronized powder) or mist.

In an embodiment, the Polypeptides of the Invention comprising e.g. at least one Nanobody and/or dAbs, preferably a Nanobody, may also form a sequence or signal that allows said Polypeptides of the Invention comprising e.g. at least one Nanobody and/or dAbs, preferably a Nanobody, to be directed towards and/or to penetrate or enter into specific gut epithelial cells, or parts or compartments of said cells, and/or that allows the Polypeptides of the Invention comprising e.g. at least one Nanobody and/or dAb, preferably a Nanobody, to penetrate or cross a biological barrier such as the gut wall or membrane.

In another preferred embodiment, the Construct of the Invention is a multispecific polypeptide comprising at least one Nanobody, domain antibody, single domain antibody or dAb directed against a target and at least one Nanobody, domain antibody, single domain antibody or dAb that directs the polypeptide of the invention towards, and/or that allows the polypeptide of the invention to penetrate or to enter into specific gut membrane cells, or parts or compartments of said cells, and/or that allows the Polypeptide of the Invention to penetrate or cross a biological barrier such as the gut wall or a cell layer of said wall, e.g. membrane.

Examples of such Nanobodies, domain antibodies, single domain antibodies or dAbs include Nanobodies, domain antibodies, single domain antibodies or dAbs that are directed towards specific cell-surface proteins, receptors, markers or epitopes of the gut membrane cells.

In this context, the Polypeptides of the Invention comprising e.g. at least one Nanobody and/or dAb, preferably a Nanobody, may comprise one or more Nanobodies, domain antibodies, single domain antibodies or dAbs directed against the desired target and one or more ligand (also called membrane crossing ligand) directed against an epithelial trans-membrane protein on the mucosal membrane, wherein said polypeptide crosses the mucosal membrane upon binding of the ligand to said epithelial trans-membrane protein.

An epithelial trans-membrane protein according to the invention is a protein or receptor displayed on the gut membrane which upon binding to a ligand mediates the transport of said ligand through the membrane.

In one embodiment of the present invention, the ligand is a Polypeptide of the Invention, e.g. a single variable domain, a Nanobody, domain antibody, single domain antibody or dAb directed against an epithelial trans-membrane protein on the gut wall, preferably the small intestine. The polypeptide or protein crosses the wall upon binding of said Nanobody, domain antibody, single domain antibody or dAb to said epithelial trans-membrane protein. The membrane crossing Nanobody, domain antibody, single domain antibody or dAb may be prepared from a peptide library which is screened for binding to the epithelial trans-membrane protein or for crossing properties. Examples of such single variable domains, e.g. Nanobodies, directed against said epithelial trans-membrane protein are the Nanobodies against FcRn, pIgR and/or VitB12 receptor as disclosed in the experimental part.

In another embodiment, the Polypeptides of the Invention comprise e.g. at least one single variable domain, a Nanobody and/or a dAb, preferably a Nanobody, and in addition a therapeutic polypeptide or agent, e.g. a Polypeptide of the Invention, e.g. against a Target Molecule, which is covalently or non-covalently linked to said single variable domain, Nanobody, domain antibody, single domain antibody or dAb that is directed against an epithelial trans-membrane protein on the gut membrane. It is an aspect of the invention that these single variable domains, Nanobodies, domain antibodies, single domain antibodies or dAbs can be added as a tag to Polypeptides of the Invention comprising e.g. at least one Nanobody and/or a dAb, preferably a Nanobody, for crossing or passage through the epithelial membrane. Examples of such a therapeutic polypeptide or agent are Nanobodies against FcRn, pIgR and/or VitB12 receptor.

In yet another embodiment, the Polypeptides of the Invention comprise e.g. at least one Nanobody and/or dAbs, preferably a Nanobody, directed against the desired Target Molecule and another ligand (e.g. a natural ligand) of the epithelial trans-membrane protein. The resulting Polypeptide, upon binding of the ligand to the epithelial trans-membrane protein, is transported through the membrane. An example of such ligand (e.g. a natural ligand) of the epithelial trans-membrane protein is the Fc unit or fragment thereof of a human antibody, e.g. the Fc unit of human IgG1.

In yet another embodiment of the present invention, the ligand is a Polypeptide of the Invention, e.g. a polypeptide comprising a single variable domain, a Nanobody, domain antibody, single domain antibody or dAb directed against an epithelial trans-membrane protein on the gut wall, preferably the small intestine, and wherein said Polypeptide of the Invention, e.g. a single variable domain, a Nanobody, domain antibody, single domain antibody or dAb directed against an epithelial trans-membrane protein on the gut wall, binds to said trans-membrane protein in a pH dependent manner, preferably binds better at acidic pH, e.g. pH 7 or less, e.g. pH5 or pH6, than at neutral physiological pH such as pH 7 or more, e.g. pH 7.4. Such pH dependent single variable domains, e.g. Nanobodies, are exemplified in this application (pH dependent human FcRn and pH dependent human serum albumin binders) and are disclosed in the experimental part.

In yet another embodiment, the Polypeptides of the Invention comprise e.g. at least one Nanobody and/or dAbs, preferably a Nanobody, directed against the desired Target Molecule and at least another single variable domain, e.g. Nanobody, domain antibody, single domain antibody or dAb that is directed against an epithelial trans-membrane protein on the gut wall, preferably the small intestine, and wherein said other single variable domain, e.g. Nanobody, domain antibody, single domain antibody or dAb binds to said trans-membrane protein in a pH dependent manner, preferably binds better at acidic pH, e.g. pH 7 or less, e.g. pH5 or pH6, than at neutral physiological pH such as pH 7 or more, e.g. pH 7.4. The resulting Polypeptide, upon binding of the ligand to the epithelial trans-membrane protein, is transported through the membrane. An example of such ligand (e.g. a natural ligand) of the epithelial trans-membrane protein is the Fc unit or fragment thereof of a human antibody, e.g. the Fc unit of human IgG1.

f) Increase of Half-Life of the Polypeptide of the Invention in Human Body, e.g. at Target Site, for e.g. Those Active Polypeptides that Require a Sustained Presence for Therapeutic Efficacy by Addition of Suitable Excipient, e.g. Biodegradable Polymer, and/or by Covalently Binding an Unit Allowing for Longer Half Life

In one specific aspect of the invention, a Polypeptide of the Invention may have an increased half-life, compared to the corresponding amino acid sequence of the invention. Some preferred, but non-limiting examples of such Polypeptides of the Invention will become clear to the skilled person based on the further disclosure herein, and for example comprise amino acid sequences that have been chemically modified to increase the half-life thereof (for example, by means of pegylation); amino acid sequences that comprise at least one additional binding site for binding to a serum protein (such as serum albumin); or amino acid sequences that is linked to at least one moiety that increases the half-life of the Polypeptide of the Invention. Examples of Polypeptides of the Invention that comprise such half-life extending moieties or amino acid sequences are clear to the skilled person; and for example include, without limitation, polypeptides in which the one or more amino acid sequences are suitable linked to one or more serum proteins or fragments thereof (such as (human) serum albumin or suitable fragments thereof) or to one or more binding units that can bind to serum proteins (such as, for example, single variable domains such as domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies that can bind to serum proteins such as serum albumin (such as human serum albumin), serum immunoglobulins such as IgG, or transferrine; reference is made to the further description and references mentioned herein, see e.g. also WO 2007/112940); polypeptides in which an amino acid sequence of the invention is linked to an Fc portion (such as a human Fc) or a suitable part or fragment thereof; or polypeptides in which the one or more amino acid sequences of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006 (see also PCT/EP2007/063348). Generally, the polypeptides of the invention with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se. In a preferred, but non-limiting aspect of the invention, such Polypeptides of the Invention has a serum half-life that is increased with more than 1 hours, preferably more than 2 hours, more preferably more than 6 hours, such as more than 12 hours, or even more than 24, 48 or 72 hours, compared to the corresponding amino acid sequence of the invention per se. In another preferred, but non-limiting aspect of the invention, such Polypeptides of the invention exhibit a serum half-life in human of at least about 12 hours, preferably at least 24 hours, more preferably at least 48 hours, even more preferably at least 72 hours or more. For example, Polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

### g) Improvement of Passive Polypeptide Transport (Diffusion) Through the Epithelial Membrane of the Intestine

The Compositions of the Invention may further comprise one or more permeation enhancer. As used herein, trans-epithelial permeation enhancers include agents which enhance the release or solubility (e.g., from a formulation delivery vehicle), diffusion rate, penetration capacity and timing, uptake, residence time, stability, effective half-life, peak or sustained concentration levels, clearance and other desired delivery characteristics (e.g. as measured at the site of delivery, or at a selected target site of activity such as the bloodstream and/or another selected physiological compartment, tissue and/or organ such as e.g. the kidney, bladder, lung and/or brain) of the Polypeptides of the Invention or of additional biologically active ingredient(s). Enhancement of passive transport through intestinal gut wall can thus occur by any of a variety of relevant mechanisms, for example by increasing the diffusion, increasing membrane fluidity, modulating the availability or action of calcium and other ions that regulate intracellular or paracellular permeation, solubilizing mucosal membrane components (e.g. lipids), changing non-protein and protein sulfhydryl levels in epithelial tissues, increasing water flux across the surface, modulating epithelial junctional physiology, reducing the viscosity of mucus overlying the epithelium, reducing mucociliary clearance rates, increasing blood flow and other mechanisms. Suitable permeability enhancing agents will be clear to a person skilled in the art of pharmacology and are further described hereafter. Such agents may be used in suitable amounts known per se, which will be clear to the skilled person based on the disclosure and prior art cited herein.

Permeability enhancing agents include (a) aggregation inhibitory agents, (b) charge modifying agents, (c) mucolytic or mucus clearing agents, (d) ciliostatic agents; (f) membrane penetration-enhancing agents such as acylcarnitine, (g) modulatory agents of epithelial junction physiology, such as nitric oxide (NO) stimulators, chitosan, and chitosan derivatives; (h) vasodilator agents, and (i) stabilizing delivery vehicles, carriers, supports or complex-forming species with which the Polypeptide of the Invention is effectively combined, associated, contained, encapsulated or bound to stabilize the Polypeptide of the Invention for enhanced intestinal transport. These agents are further exemplified—without being limiting as additional agents comprised in the compositions of the present invention—in WO98034632C2, WO98034632, WO9834632, WO9834632, WO9736480 and/or WO9630036.

In a further embodiment, a membrane penetration-enhancing agent is added to the composition of the present invention. Different membrane penetration-enhancing agents have been described such as (i) a surfactant, (ii) a bile salt or bile salt derivative, (iii) a phospholipid or fatty acid additive, mixed micelle, liposome, or carrier, (iv) an alcohol, (v) an enamine, (vi) an NO donor compound, (vii) a long-chain amphipathic molecule (viii) a small hydrophobic penetration enhancer, (ix) sodium or a salicylic acid derivative, (x) a glycerol ester of acetoacetic acid, (xi) a cyclodextrin or beta-cyclodextrin derivative, (xii) a medium-chain fatty acid, (xiii) a chelating agent (e.g., citric acid, salicylates), (xiv) an amino acid or salt thereof, (xv) an N-acetylamino acid or salt thereof, (xvi) an enzyme degradative to a selected membrane component, (xvii) an inhibitor of fatty acid synthesis, (xviii) an inhibitor of cholesterol synthesis, (xix) cationic polymers, or (xx) any combination of the membrane penetration enhancing agents of ((i)-(xix)). The membrane penetration-enhancing agent can be selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium lauryl sulfate, oleic acid, and the bile salts (e.g., unsaturated cyclic ureas and Transcutol), may be employed to enhance mucosal penetration of the Nanobodies, polypeptides or proteins of the invention. In additional aspects, surfactants (e.g., Tween 80, Poloxamer 188, polysorbates; further non-limiting examples of surfactants are also provided in EP 490806, U.S. Pat. No. 5,759,565, and WO 04/093917) are employed as adjunct compounds, processing agents, or formulation additives to enhance oral delivery of the Nanobodies, polypeptides or proteins of the invention. These penetration-enhancing agents typically interact at either the polar head groups or the hydrophilic tail regions of molecules that comprise the lipid bilayer of epithelial cells lining the oralmucosa (Barry, Pharmacology of the Skin, Vol. 1, pp. 121-137, Shroot et al., Eds., Karger, Basel, 1987; and Barry, J. Controlled Release 1987; 6: 85-97). Interaction at these sites may have the effect of disrupting the packing of the lipid molecules, increasing the fluidity of the bilayer, and facilitating transport of the Polypeptides of the Invention across the mucosal barrier. Additional non-limiting examples of membrane penetration-enhancing agent are described in WO 04/093917, WO 05/120551 and Davis and Illum (Clin. Pharmacokinet 2003, 42: 1107-1128).

In various embodiments of the invention, the Polypeptide of the Invention is combined with one, two, three, four or more of the permeability enhancing agents recited in (a)-(k) above. These agents may be admixed, alone or together, with the oralcarrier and with the Polypeptide of the Invention, or otherwise combined therewith in a pharmaceutically acceptable formulation or delivery vehicle.

While the mechanism of absorption promotion may vary with different permeability-enhancing agents of the invention, useful reagents in this context will not substantially adversely affect the tissue and will be selected according to the physicochemical characteristics of the particular Polypeptide of the Invention or other active ingredients or delivery enhancing agent. In this context, delivery-enhancing agents that increase penetration or permeability of the gut wall will often result in some alteration of the protective permeability barrier of the gut. For such delivery-enhancing agents to be of value within the invention, it is generally desired that any significant changes in permeability of the gut be reversible within a time frame appropriate to the desired duration of drug delivery. Furthermore, there should be no substantial, cumulative toxicity, nor any permanent deleterious changes induced in the barrier properties of the gut with long term use.

Some preferred embodiments are using the above disclosed strategies are provided below:

In one of the embodiments of the Polypeptides of the Invention, e.g. Nanobodies or dAbs, preferably Nanobodies, more preferably agonistic polypeptides, systemic and/or local (i.e. topical gut) delivery, is provided through oral administration by protecting said polypeptides from proteolytic degradation by e.g. enteric coatings known to the skilled person in the art.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art; and b) improving active (e.g. receptor mediated) trans-epithelial transport of said polypeptides, e.g. by pIgR, FcRn, and/or VitB12 receptor mediated trans-epithelial transport, preferably pIgR and/or FcRn, more preferably FcRn mediated trans-epithelial transport.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art; and b) improving active (e.g. receptor mediated) trans-epithelial transport of said polypeptides, e.g. by pIgR, FcRn, and/or VitB12 receptor mediated trans-epithelial transport, preferably pIgR and/or FcRn, more preferably FcRn mediated trans-epithelial transport; and c) increasing half-life of the polypeptide in human body, e.g. at target site, for e.g. those active polypeptides that require a sustained presence for therapeutic efficacy by addition of a suitable excipient, e.g. biodegradable polymer, and/or by covalently binding an unit allowing for longer half life, e.g. fused Fc fragment, albumin, albumin binder, FcRn binder, and/or serum protein binder. In a preferred embodiment, the unit extending half-life is also able to improve active (e.g. receptor mediated) trans-epithelial transport of said polypeptides, e.g. a FcRn binding unit is able to prolong half/life and improve active receptor mediated trans-epithelial transport in the gut.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled, person in the art; and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH5 or pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art; and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH5 or pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport; and c) increasing half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life, e.g. fused Fc fragment, albumin, albumin binder, FcRn binder, and/or serum protein binder. In a preferred embodiment, the unit extending half-life is also able to improve active (e.g. receptor mediated) trans-epithelial transport of said polypeptides, e.g. a FcRn binding unit is able to prolong half/life and improve active receptor mediated trans-epithelial transport in the gut.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art; and b) develop protease-resistant polypeptide analogs that retain biological activity, e.g. pharmaceutical oral compositions comprising Target Molecule binding single variable domains, e.g. Nanobodies or dAbs, selected for protease resistance by at least 2, 3, 4, 5 10, 20, 50 100 folds (see e.g. experimental part); c) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH5 or pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport; and d) increasing half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life, e.g. fused Fc fragment, albumin, albumin binder, FcRn binder, and/or serum protein binder. In a preferred embodiment, the unit extending half-life is also able to improve active (e.g. receptor mediated) trans-epithelial transport of said polypeptides, e.g. a FcRn binding unit is able to prolong half/life and improve active receptor mediated trans-epithelial transport in the gut.

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art; and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport; and [c) inhibit proteolytic activity that degrades polypeptides in stomach and gut by e.g. protease inhibitors such as e.g. organic acids; and/or d) improve passive polypeptide transport (diffusion) through the mucus and epithelial membrane by e.g. permeation enhancer such as acylcarnitine and/or Eligen® carrier technology].

In another embodiment of the Polypeptides of the Invention, e.g. Nanobodies or dAbs, preferably Nanobodies, systemic and/or local (i.e. topical gut) delivery is provided through oral administration provided by a) protecting said polypeptides from proteolytic degradation by e.g. enteric coatings known to the skilled person in the art; and b) improving active (e.g. receptor mediated) trans-epithelial transport of said polypeptides, wherein said receptor binding is a high affinity binding (e.g. dissociation constant of 100 nM, preferably 10 nM, more preferably 1 nM or 100 pM, most preferred 10 pM, at pH6 or less but has 2 times less, preferably 3, 4, 5, 10, 20, 50 or 100 times less, more preferably no binding at pH7 and more, e.g. by pH dependent pIgR, pH dependent FcRn, and/or pH dependent VitB12 receptor mediated trans-epithelial transport, preferably pIgR and/or FcRn, more preferably FcRn mediated trans-epithelial transport; and c) increasing half-life of the polypeptide in human body, e.g. at target site, for e.g. those active polypeptides that require a sustained presence for therapeutic efficacy by addition of suitable excipient, e.g. biodegradable polymer, and/or by covalently binding an unit allowing for longer half life, e.g. fused Fc fragment, albumin, albumin binder, FcRn binder, and/or serum protein binder; and [d) inhibit proteolytic activity that degrades polypeptides in stomach and gut by e.g. protease inhibitors such as e.g. organic acids; and/or e) improve passive polypeptide transport (diffusion) through the mucus and epithelial membrane by e.g. permeation enhancer such as acylcarnitine and/or Eligen® carrier technology].

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art; and b) improving active (e.g. receptor mediated) trans-epithelial transport of said polypeptides, e.g. by pIgR, FcRn, and/or VitB12 receptor mediated trans-epithelial transport, preferably pIgR and/or FcRn, more preferably FcRn mediated trans-epithelial transport; and [c) inhibit proteolytic activity that degrades polypeptides in stomach and gut by e.g. protease inhibitors such as e.g. organic acids; and/or d) improve passive polypeptide transport (diffusion) through the mucus and epithelial membrane by e.g. permeation enhancer such as acylcarnitine and/or Eligen® carrier technology].

In another embodiment of the Polypeptides of the Invention, e.g. Nanobodies or dAbs, preferably Nanobodies, systemic and/or local (i.e. topical gut) delivery is provided through oral administration by a) protecting said polypeptides from proteolytic degradation by e.g. enteric coatings known to the skilled person in the art; and b) providing continuous local (topical in gut) delivery by bacterial system, e.g. lactic acid bacteria.

### Further Embodiments

Moreover, in one embodiment, anti-aggregation agents are added to the composition of the invention. Aggregation inhibitory agents include, for example, polymers of various functionalities, such as polyethylene glycol, dextran, diethylaminoethyl dextran, and carboxymethyl cellulose, which significantly increase the stability and reduce the solid-phase aggregation of polypeptides admixed therewith or linked thereto. In some instances, the activity or physical stability of polypeptides can also be enhanced by various additives to pharmaceutical compositions comprising the Polypeptide of the Invention. For example, additives, such as polyols (including sugars), amino acids, and various salts may be used. Certain additives, in particular sugars and other polyols, also impart significant physical stability to dry, e.g., lyophilized polypeptides. These additives can also be used within the invention to protect the polypeptides against aggregation not only during lyophilization but also during storage in the dry state. For example, sucrose and Ficoll 70 (a polymer with sucrose units) exhibit significant protection against polypeptide aggregation during solid-phase incubation under various conditions. These additives may also enhance the stability of solid polypeptides embedded within polymer matrices. Yet additional additives, for example sucrose, stabilize polypeptides against solid-state aggregation in humid atmospheres at elevated temperatures, as may occur in certain sustained-release formulations of the invention. These additives can be incorporated into polymeric melt processes and compositions within the invention. For example, polypeptide microparticles can be prepared by simply lyophilizing or spray drying a solution containing various stabilizing additives described above. Sustained release of unaggregated polypeptides can thereby be obtained over an extended period of time. A wide non-limiting range of suitable methods and anti-aggregation agents are available for incorporation within the compositions of the invention such as disclosed in WO 05/120551, Breslow et al. (J. Am. Chem. Soc. 1996; 118: 11678-11681), Breslow et al. (PNAS USA 1997; 94: 11156-11158), Breslow et al. (Tetrahedron Lett. 1998; 2887-2890), Zutshi et al. (Curr. Opin. Chem. Biol. 1998; 2: 62-66), Daugherty et al. (J. Am. Chem. Soc. 1999; 121: 4325-4333), Zutshi et al. (J. Am. Chem. Soc. 1997; 119: 4841-4845), Ghosh et al. (Chem. Biol. 1997; 5: 439-445), Hamuro et al. (Angew. Chem. Int. Ed. Engl. 1997; 36: 2680-2683), Alberg et al., Science 1993; 262: 248-250), Tauton et al. (J. Am. Chem. Soc. 1996; 118: 10412-10422), Park et al. (J. Am. Chem. Soc. 1999; 121: 8-13), Prasanna et al. (Biochemistry 1998; 37:6883-6893), Tiley et al. (J. Am. Chem. Soc. 1997; 119: 7589-7590), Judice et al. (PNAS USA 1997; 94: 13426-13430), Fan et al. (J. Am. Chem. Soc. 1998; 120: 8893-8894), Gamboni et al. (Biochemistry 1998; 37: 12189-12194).

In another embodiment, enzyme inhibitors are added to the composition of the invention. The stomach and gut contain hydrolytic enzymes, such as lipases and proteases, which must be overcome. This enzymatic “barrier” can be dampened by administering enzyme inhibitors that prevent or at least lessen the extent of degradation. Enzyme inhibitors for use within the invention are selected from a wide range of non-protein inhibitors that vary in their degree of potency and toxicity (see, e.g., L. Stryer, Biochemistry, WH: Freeman and Company, NY, N.Y., 1988). Non-limiting examples include amastatin and bestatin (O'Hagan et al., Pharm. Res. 1990, 7: 772-776). Various classes of enzyme inhibitors are extensively described and exemplified in WO 05/120551 without being limiting for use in the composition of the present invention. Another means to inhibit degradation is pegylation with PEG molecules, preferably low molecular weight PEG molecules (e.g. 2 kDa; Lee et al., Calcif Tissue Int. 2003, 73: 545-549). Also within the scope of the present invention is the use, as enzyme inhibitor, of a Nanobody, domain antibody, single domain antibody or “dAb” directed against said enzyme. Accordingly, the invention also relates to a bispecific or multispecific Polypeptide comprising or essentially consisting of one or more Nanobodies, domain antibodies, single domain antibodies or “dAbs” directed against the desired target and one or more Nanobodies, domain antibodies, single domain antibodies or “dAbs” directed against an enzyme of the stomach and/or gut.

In addition to the Polypeptide of the Invention and, optionally, one or more additives and/or agents, the composition of the invention may further comprise one or more additional therapeutic ingredients (or active substances). These therapeutic ingredients can be any compound that elicits a desired activity or therapeutic or biological response in the subject. In a preferred embodiment, two or more Nanobodies the invention may be used in combination, i.e. as a combined treatment regimen.

As indicated above, the pharmaceutical composition of the invention should comprise at least a therapeutically effective amount of the Polypeptide of the Invention, e.g. the polypeptides comprising single variable domains, e.g. Nanobodies. A “therapeutically effective amount” as used in the present invention in its broadest sense means an amount of the Polypeptide of the Invention that is capable of eliciting the desired activity or the desired biological, prophylactic and/or therapeutic response. The amount of Polypeptide of the Invention to be administered and hence the amount of active ingredient in the pharmaceutical composition of the invention will, of course, vary according to factors such as the bioavailability of the polypeptide, the disease indication and particular status of the subject (e.g., the subject's age, size, fitness, extent of symptoms, susceptibility factors, etc), the target cell, tumor, tissue, graft or organ, other drugs or treatments being administered concurrently, as well as the specific pharmacology of the Polypeptides of the Invention for eliciting the desired activity or biological, prophylactic or therapeutic response in the subject. Dosage regimens may be adjusted to provide an optimum activity or biological, prophylactic or therapeutic response. Dosages should also be adjusted based on the release rate of the administered formulation (e.g. a slow release polymer containing composition versus a capsule comprising pressed Polypeptide of the Invention). A therapeutically effective amount is also one in which any toxic or detrimental side effects of the Polypeptide of the Invention are outweighed in clinical terms by therapeutically beneficial effects. Doses may be chosen to be equipotent to the injection route.

In this context, the absolute bioavailability of the Polypeptide of the Invention following oral administration of the Pharmaceutical Composition of the Invention is of the order of ca. 1, 2, 3, 5, 7, 10, 15, 20, 25, 30, 40, 50, 60, 70, 80, 90, 100% or more of the levels achieved with the corresponding injection. Absolute bioavailability measures the availability of the active drug in systemic circulation after oral administration when compared with intravenous administration. The absolute bioavailability of the Polypeptides of the Invention is determined by comparing the concentration vs. time plot of the Polypeptides of the Invention after intravenous (IV) administration with the concentration vs. time plot of the Polypeptides of the Invention after oral (IN) administration. The absolute bioavailability of Polypeptides of the Invention is defined as (AUC_{IN}×dose_{IN})/(AUC_{IV}×dose_{IV})×100.

The relative bioavailability of the Polypeptides of the Invention following oral administration of the Pharmaceutical Composition of the Invention is of the order of ca. 1, 2, 3, 5, 7, 10, 15, 20, 25, 30, 40, 50, 60, 70, 80, 90, 100% or more of the levels achieved with the corresponding injection. Relative bioavailability measures the availability of the active drug in systemic circulation after oral administration when compared with another form of administration of the same drug, such as intramuscular (IM) or subcutaneous (SC). The relative bioavailability of Polypeptides of the Invention is determined by comparing the concentration vs. time plot of Polypeptides of the Invention after intramuscular (IM) or subcutaneous (SC) administration with the concentration vs. time plot of Polypeptides of the Invention after oral (IN) administration. The relative bioavailability of Polypeptides of the Invention is defined as (AUC_{IN}×dose_{IN})/(AUC_{SC/IM}×dose_{SC/IM})×100. Accordingly, in order to be equipotent to the injection route, oral administration will appropriately be effected so as to give a dosage rate of the order of 1 to 100 times, preferably 1 to 50 times, more preferably 1 to 20 times, even more preferably 1 to 10 times the dosage required for treatment via injection, also depending on the frequency of the oral application.

The amount of active compound will generally be chosen to provide effective treatment on administration once a day or once a week or once a month. Alternatively, dosages may be split over a series of e.g. 1 to 4 applications taken at intervals during the day, week or month. The sub-dose itself may be further divided, e.g., into a number of discrete loosely spaced administrations; such as multiple takings of a plurality of pills or capsules. To maintain more consistent or normalized therapeutic levels of the Polypeptide of the Invention, it may be advisable that the Composition of the Invention is repeatedly administered to the subject, for example one, two or more times within a 24 hour period, four or more times within a 24 hour period, six or more times within a 24 hour period, or eight or more times within a 24 hour period. An administration regimen could include long-term, daily, weekly or monthly treatment. By “long-term” is meant at least two weeks and preferably, several weeks, months, or years of duration. The clinician will generally be able to determine a suitable daily, weekly or monthly dose, depending on the factors mentioned herein. It will also be clear that in specific cases, the clinician may choose to deviate from these amounts, for example on the basis of the factors cited above and his expert judgment.

The final determination of the effective dosage will be based on animal model studies, followed up by human clinical trials, and is guided by determining effective dosages and oral administration protocols that significantly reduce the occurrence or severity of the targeted disease symptoms or conditions in the subject. Suitable models in this regard include, for example, murine, rat, porcine, feline, non-human primate, and other accepted animal model subjects known in the art. Ultimately, the dosage of Polypeptides of the Invention will be at the discretion of the attendant, physician or clinician. The dosage can also be adjusted by the individual physician in the event of any complication.

As a non-limiting example, the Polypeptides of the Invention is suitably presented in the Pharmaceutical Composition of the Invention in an amount such as to provide a free Polypeptides of the Invention concentration from about 0.1 microgram to 0.1 gram per kg body weight per day, such as from 1 microgram to 0.1 gram per kg body weight per day, such as from 0.01 to 100 milligram per kg body weight per day, such as from 0.05-100 milligram, such as from 0.05 to 50 milligram, 0.05 to 30 milligram, 0.1 to 20 milligram, or from about 1 to 10 or about 5 to 10 milligram per kg body weight per day either as a single daily dose or as multiple divided doses during the day.

The proportion of each further component in the oral composition of the invention may vary depending on the components used. For example, but without being limiting, the amount of enteric coating may be in the range of from 0.1 to 99.9%, preferably 1 to 20% by weight of the total weight of the composition. When present, the amount of permeability enhancer may be in the range from about 0.01 to about 10% or higher and preferably about 0.05 to about 1.0% by weight of the total weight of the composition, the amount depending on the specific enhancer used. The amount is generally kept as low as possible since above a certain level no further enhancement of absorption can be achieved and also too high of a enhancer level may cause irritation of the gut. The amount of protease inhibitor may be at least 0.1%, suitably in the range from about 0.5 to 10% of the total weight of the composition. Preserving agents may be present in an amount of from about 0.002 to 0.02% by weight of the total weight or volume of the composition. The amount of the other excipients will be determined by processes known to the skilled person in the art.

In addition to the concentration of the different compounds in the composition of the invention, the total delivery weight is important to consider as well. The delivery weight is relatively high for oral compositions and may be up to 1 g or more. Suitable delivery weights will be clear to a person skilled in the art of pharmacology.

The present invention further provides a method for the preparation of a composition mixing the Polypeptides of the Invention, e.g. the single variable domains, Nanobodies, the domain antibodies, the single domain antibodies or the dAbs and the pharmaceutically acceptable excipients (as proposed herein, e.g. protease inhibitors, slow release matrices, and/or permeability enhancer) and thus resulting in a powder that is then further e.g. filled into capsules, preferably enterically coated capsules. Alternatively, said powder comprising the Polypeptides of the Invention and the excipients are milled into smaller granules (dry or wet granulation) and pressed into the core pill—said core pill is then further coated e.g. by enteric coating. All above described steps may be prepared in a conventional manner known to the skilled person in pharmacology.

The solid oral composition of the invention may be prepared in conventional manner. E.g. the Polypeptides of the Invention, e.g. Nanobodies, may be admixed with the protease inhibitors, slow release matrices, and/or permeability enhancer, optionally with further ingredients, additives and/or agents as indicated above. The Polypeptides of the Invention, e.g. Nanobodies, may be in solution e.g. an aqueous or alcoholic solution when being mixed with the protease inhibitors, slow release matrices, and/or permeability enhancer and the solvent evaporated, e.g. under freeze-drying or spray drying. Such drying may be effected under the conventional conditions. Alternatively the dry mixtures may be compacted and/or granulated and then be pulverized and/or sieved. If desired the compacted composition may be further coated. According to a preferred embodiment of the invention, the oral composition is prepared by lyophilisation, then granulated and filled up into enterically coated capsules. A homogeneous solution, preferably aqueous, containing the Polypeptides of the Invention, e.g. Nanobodies, and optionally containing further ingredients, additives and/or agents as discussed above, e.g. protease inhibitors, slow release matrices, and/or permeability enhancer, is prepared and then submitted to lyophilisation in analogy with known lyophilisation procedures, and to subsequent drying. The resulting powder may then be filled up into enterically coated capsules before administration.

Alternatively, the Polypeptides of the Invention may be administered in liquid form such as in the form of a suspension or partly or fully dissolved solution, e.g. the lyophilized powder may be reconstituted in e.g. water before administration or may be stored in liquid form and thus may be directly be used as such.

For administration of a liquid, for example, such compositions will suitably be put up in a container provided with a conventional dropper/closure device, e.g. comprising a pipette or the like, preferably delivering a substantially fixed volume of composition/drop.

If desired a powder or liquid may be filled into a soft or hard capsule adapted for oral administration. The powder may be sieved before filled into the capsules such as gelatine capsules, preferably an enterically coated capsule.

The Pharmaceutical Composition of the Invention is formulated for oral administration and for delivery of the Polypeptides of the Invention (at least the therapeutically active moiety) either locally to the gut and/or systemically to the body providing a systemic therapeutic or biological response of the Polypeptides of the Invention, e.g. the Nanobodies, in the subject. This means that there is a sufficient amount of functional (i.e. active or not inactivated) Polypeptide of the Invention, e.g. the Nanobodies, present in the blood (and/or another selected physiological compartment, tissue and/or organ such as e.g. the kidney, bladder and/or lung) to provide the desired therapeutic effect (i.e. to elicit the desired activity or the desired biological, prophylactic or therapeutic response in the subject receiving said Polypeptide of the Invention, e.g. the Nanobodies. The bioavailability of the Polypeptides of the Invention, e.g. the Nanobodies, in the blood (and/or another selected physiological compartment, tissue and/or organ such as e.g. the kidney, bladder and/or lung) and/or in the brain following administration of the composition of the invention is determined by measuring the pharmacokinetic parameters Cmax (peak concentration), AUC (area under concentration vs. time curve) and/or Tmax (time to maximal blood concentration), which are well known to those skilled in the art (Laursen et al., Eur. J. Endocrinology, 1996; 135: 309-315). The bioavailability of the Polypeptide of the Invention, e.g. the Nanobodies, may be determined in any conventional mariner, e.g. by radioimmunoassay.

“Cmax”, as used in the present invention, is the mean maximum concentration of the Polypeptide of the Invention achieved in blood (and/or another selected physiological compartment, tissue and/or organ such as e.g. the kidney, bladder and/or lung), following oral administration of a single dosage of the pharmaceutical composition to the subject. Blood or bloodstream as used in the present invention, can be any form and/or fraction of blood. Without being limiting, blood or bloodstream includes plasma and/or serum. The Cmax for the Polypeptide of the Invention comprised in the pharmaceutical composition of the invention can have any value as long as said Polypeptide of the Invention provides the desired activity or therapeutic or biological response in the subject in need of said Polypeptide of the Invention, e.g. the Nanobodies. In an embodiment of the invention, the Polypeptide of the Invention reaches a Cmax in blood of at least 1 ng of Polypeptide of the Invention per ml of blood. In a further embodiment, the Polypeptide of the Invention reaches a Cmax in blood of at least 2, 5, 10, 15, 20, 30, 40, 50, 100, 150, 200, 300, 400, 500, 750, 100 ng or more of Polypeptide of the Invention, e.g. the Nanobodies, per ml of blood.

In another embodiment, the Polypeptide of the Invention, reaches a Cmax in blood of at least 1 ng of Polypeptide of the Invention, per ml of blood following oral administration of a dose of 5 mg/kg body weight of said Polypeptide of the Invention, e.g. the Nanobodies. In a further embodiment, the Polypeptide of the Invention, reaches a Cmax in blood of at least 2, 5, 10, 15, 20, 30, 40, 50, 100, 150, 200, 300, 400, 500, 750, 100 ng or more of Polypeptide of the Invention, per ml of blood following oral administration of a dose of 5 mg/kg body weight of said Polypeptide of the Invention.

In another embodiment of the invention, following oral administration of Polypeptide of the Invention, said polypeptide reaches a Cmax in blood of at least 1% of the Cmax that is reached following parenteral administration of the same amount of the Polypeptide of the Invention. In a further embodiment, following oral administration of the Polypeptide of the Invention, said Polypeptide of the Invention reaches a Cmax in blood of at least 2, 3, 5, 7, 10, 15, 20, 25, 30, 40, 50% or more of the Cmax that is reached following parenteral administration of the same amount of Polypeptide of the Invention.

“Tmax”, as used in the present invention, is the mean time to reach maximum concentration of the Polypeptide of the Invention in blood (and/or another selected physiological compartment, tissue and/or organ such as e.g. the kidney, bladder and/or lung) following oral administration of a single dosage of the composition of the invention. The Tmax for the Polypeptide of the Invention comprised in the composition of the invention can have any value as long as said Polypeptide of the Invention provides the desired activity or therapeutic or biological response in the subject in need of said Polypeptide of the Invention. In an embodiment of the invention, the Polypeptide of the Invention reaches the bloodstream with a Tmax of less than 120 minutes. In a further embodiment, the Polypeptide of the Invention reaches the bloodstream with a Tmax of less than 90, 60, 50, 40, 30, 20, 10, or 5 minutes. In a further embodiment, the Polypeptide of the Invention reaches the brain with a Tmax of less than 90, 60, 50, 40, 30, 20, 10, or 5 minutes.

The “concentration vs. time curve” measures the concentration of the Polypeptide of the Invention in blood (and/or another selected physiological compartment, tissue and/or organ such as e.g. the kidney, bladder and/or lung) of a subject vs. time after administration of a dosage of the composition of the invention.

In an embodiment, the Polypeptide of the Invention reaches a Cmax in blood of at least 1 ng of Polypeptide of the Invention per ml of blood within less than 120 minutes following oral administration of the composition of the invention. In a further embodiment, the Polypeptide of the Invention reaches a Cmax in blood of at least 2, 5, 10, 15, 20, 30, 40, 50, 100, 150, 200, 300, 400, 500, 750, 1000 ng or more of Polypeptide of the Invention per ml of blood within less than 120 minutes following oral administration of the composition of the invention. In another embodiment, the Polypeptide of the Invention reaches a Cmax in blood of at least 1 ng of Polypeptide of the Invention per ml of blood within less than 90, 60, 50, 40, 30, 20, 10, or 5 minutes following oral administration of the composition of the invention. In a further embodiment, the Polypeptide of the Invention reaches a Cmax in blood of at least 2, 5, 10, 15, 20, 30, 40, 50, 100, 150, 200, 300, 400, 500, 750, 1000 ng or more of Polypeptide of the Invention per ml of blood within less than 90, 60, 50, 40, 30, 20, 10, or 5 minutes following oral administration of the composition of the invention.

In another embodiment, the Polypeptide of the Invention reaches a Cmax in blood of at least 1 ng of Polypeptide of the Invention per ml of blood within less than 120 minutes following oral administration of a dose of 5 mg/kg body weight of said Polypeptide of the Invention. In a further embodiment, the Polypeptide of the Invention reaches a Cmax in blood of at least 2, 5, 10, 15, 20, 30, 40, 50, 100, 150, 200, 300, 400, 500, 750, 1000 ng or more of the Polypeptide of the Invention per ml of blood within less than 120 minutes following oral administration of a dose of 5 mg/kg body weight of said Polypeptide of the Invention. In another embodiment, the Polypeptide of the Invention reaches a Cmax in blood of at least 1 ng of the Polypeptide of the Invention per ml of blood within less than 90, 60, 50, 40, 30, 20, 10, or 5 minutes following oral administration of a dose of 5 mg/kg body weight of said Polypeptide of the Invention. In a further embodiment, the Polypeptide of the Invention reaches a Cmax in blood of at least 2, 5, 10, 15, 20, 30, 40, 50, 100, 150, 200, 300, 400, 500, 750, 1000 ng or more of Polypeptide of the Invention per ml of blood within less than 90, 60, 50, 40, 30, 20, 10, or 5 minutes following oral administration of a dose of 5 mg/kg body weight of said Polypeptide of the Invention.

The “area under the curve (AUC)”, as used in the present invention, is the area under the curve in a plot of concentration of the Polypeptide of the Invention in blood (and/or another selected physiological compartment, tissue and/or organ such as e.g. the kidney, bladder and/or lung) against time. Mathematically, this value is a measure of the integral of the instantaneous concentrations during a time interval. AUG is usually given for the time interval zero to infinity, and other time intervals are indicated (for example AUC (t₁,t₂) where t₁ and t₂ are the starting and finishing times for the interval). Clearly blood (and/or another selected physiological compartment, tissue and/or organ such as e.g. the kidney, bladder and/or lung) Polypeptide of the Invention concentrations cannot be measured to ‘infinity’ for a subject so mathematical approaches are used to estimate the AUC from a limited number of concentration measurements. The AUC (from zero to infinity) is used to measure the total amount of Polypeptide of the Invention absorbed by the body, irrespective of the rate of absorption. This is useful when trying to determine whether two application formulations with the same dose (for example parenteral and oral) release the same dose of Polypeptide of the Invention to the body.

The AUC for the Polypeptide of the Invention comprised in the composition of the invention can have any value as long as said Polypeptide of the Invention provides the desired activity or biological response in the subject in need of said Polypeptide of the Invention. In an embodiment of the invention, the AUC for the Polypeptide of the Invention in blood following oral administration of a composition comprising said Polypeptide of the Invention is at least 500 ng/ml/minute of the Polypeptide of the Invention. In a further embodiment, the AUC for the Polypeptide of the Invention in blood following oral administration of a composition comprising said Polypeptide of the Invention is at least 600, 700, 800, 900, ng/ml/minute or at least 1, 1.5, 2, 3, 4, 5, 10 or 15 μg/ml/minute of the Polypeptide of the Invention.

In another embodiment of the invention, the AUC for the Polypeptide of the Invention in blood following oral administration of a dose of 5 mg/kg body weight of said Polypeptide of the Invention is at least 500 ng/ml/minute Polypeptide of the Invention. In a further embodiment, the AUC for the Polypeptide of the Invention in blood following oral administration of a dose of 5 mg/kg body weight of said Polypeptide of the Invention is at least 600, 700, 800, 900 ng/ml/minute or 1, 1.5, 2, 3, 4, 5 or 10 μg/ml/minute Polypeptide of the Invention per ml of blood.

As discussed above, in an embodiment of the invention, the bioavailability (absolute or relative) for the Polypeptide of the Invention in blood following oral administration of a composition comprising said Polypeptide of the Invention is at least 1% compared to parenteral administration of said Polypeptide of the Invention. In a further embodiment, the bioavailability for the Polypeptide of the Invention in blood following oral administration of a composition comprising said Polypeptide of the Invention is at least 2, 3, 5, 7, 10, 15, 20, 25, 30, 40, 50, 60, 70, 80, 90, 100% or more compared to parenteral administration of said Polypeptide of the Invention. Preferably the bioavailability (absolute or relative) for the Polypeptide of the Invention in blood following oral administration of a composition comprising said Polypeptide of the Invention is at least 5% compared to parenteral administration of said Polypeptide of the Invention.

Oral administration of one or more Polypeptides of the Invention to a subject yields effective delivery of the Polypeptides of the Invention to the blood (and/or another selected physiological compartment, tissue and/or organ such as e.g. the kidney, bladder and/or lung) to elicit the desired activity or therapeutic or biological response in the subject. In a preferred embodiment of the invention, the Polypeptide of the Invention provides the prevention and/or treatment of a selected disease or condition in said subject. Accordingly, another aspect of the invention relates to a method for the prevention and/or treatment of a subject in need of a Polypeptide of the Invention, said method comprising orally administering, to said subject a therapeutically effective amount of said Polypeptide of the Invention, and/or of a composition comprising the same.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk from, the diseases and/or disorder.

The invention also relates to a method for the prevention and/or treatment of at least one disease or disorder that can be prevented and/or treated by administering a Polypeptide of the Invention to a subject suffering from said disease or disorder, said method comprising orally administering to said subject a therapeutically effective amount of the Polypeptide of the Invention, and/or of a composition comprising the same. Accordingly, the invention relates to the Polypeptides or compositions of the invention for the prevention and/or treatment of at least one disease or disorder that can be prevented and/or treated by orally administering to a subject a Polypeptide of the Invention.

In another embodiment, the invention relates to a method for immunotherapy, and in particular for passive immunotherapy, which method comprises oral administering, to a subject suffering from or at risk of a diseases and/or disorders that can be cured or alleviated by immunotherapy with a Polypeptide of the Invention, a therapeutically effective amount of said Polypeptide of the Invention and/or of a composition comprising the same.

The polypeptides present in the compositions of the invention may be directed against any suitable target that is of therapeutic or diagnostic interest. The polypeptides can be functional as agonists as well as antagonists, preferably agonists. Examples include but are not limited to targets of therapeutic interests such as EPO, Growth Hormone, TNF-α, IgE, IFN-γ, MMP-12, EGFR, CEA, H. pylori, M. tuberculosis, influenza, β-amyloid, vWF, IL-6, IL-6R, PDK1, CD40, OVA, VSG, S. typhimurium, Rotavirus, Brucella, parathyroid hormone-derived peptides.

The invention provides systemic delivery of the Polypeptide of the Invention. The desired target can be a target in any physiological compartment, tissue or organ. In an embodiment, the Polypeptide of the Invention is directed against a target in the kidney or the bladder and the invention relates to a method for the prevention and/or treatment of a subject in need of a Polypeptide of the Invention that is directed against a target in the kidney or bladder, said method comprising orally administering, to said subject a therapeutically effective amount of said Polypeptide of the Invention, and/or of a composition comprising the same. The invention also relates to a method for the prevention and/or treatment of at least one disease or disorder that can be prevented and/or treated by administering to a subject suffering from said disease or disorder a Polypeptide of the Invention that is directed against a target in the kidney or the bladder, said method comprising orally administering to said subject a therapeutically effective amount of said Polypeptide of the Invention, and/or of a composition comprising the same. The invention also relates to a method for the prevention and/or treatment of a disease or disorder of the kidney or bladder, said method comprising orally administering to said subject a therapeutically effective amount of a Polypeptide of the Invention that is directed against a target in the kidney or the bladder and/or of a composition comprising the same. Accordingly, the invention also relates to the composition of the invention, wherein the Polypeptide of the Invention is directed against a target in the kidney or the bladder for the prevention and/or treatment of a disease or disorder of the kidney or bladder.

In another embodiment, the Polypeptide of the Invention is directed against a target in the lung and the invention relates to a method for the prevention and/or treatment of a subject in need of a Polypeptide of the Invention that is directed against a target in the lung, said method comprising orally administering, to said subject a therapeutically effective amount of said Polypeptide of the Invention, and/or of a composition comprising the same. The invention also relates to a method for the prevention and/or treatment of at least one disease or disorder that can be prevented and/or treated by administering to a subject suffering from said disease or disorder a Polypeptide of the Invention that is directed against a target in the lung, said method comprising orally administering to said subject a therapeutically effective amount of said Polypeptide of the Invention, and/or of a composition comprising the same. The invention also relates to a method for the prevention and/or treatment of a disease or disorder of the lung, said method comprising orally administering to said subject a therapeutically effective amount of a Polypeptide of the Invention that is directed against a target in the lung and/or of a composition comprising the same. Accordingly, the invention also relates to the composition of the invention, wherein the Polypeptide of the Invention is directed against a target in the lung for the prevention and/or treatment of at least one disease or disorder of the lung.

In another preferred embodiment, the Polypeptide of the Invention is directed against a target on a tumor cell and the invention relates to a method for the prevention and/or treatment of a subject in need of a Polypeptide of the Invention that is directed against a target on a tumor cell, said method comprising orally administering, to said subject a therapeutically effective amount of said Polypeptide of the Invention, and/or of a composition comprising the same. The invention also relates to a method for the prevention and/or treatment of at least one disease or disorder that can be prevented and/or treated by administering to a subject suffering from said disease or disorder a Polypeptide of the Invention that is directed against a target on a tumor cell, said method comprising orally administering to said subject a therapeutically effective amount of said Polypeptide of the Invention, and/or of a composition comprising the same. The invention also relates to a method for the prevention and/or treatment of a tumor related disease or disorder, said method comprising orally administering to said subject a therapeutically effective amount of a Polypeptide of the Invention that is directed against a target on a tumor and/or of a composition comprising the same. Accordingly, the invention also relates to the composition of the invention, wherein the Polypeptide of the Invention is directed against a target on a tumor for the prevention and/or treatment of at least one a tumor related disease or disorder.

In another embodiment, the Polypeptide of the Invention is directed against TNF and the invention relates to a method for the prevention and/or treatment of a subject in need of a Polypeptide of the Invention that is directed against TNF, said method comprising orally administering, to said subject a therapeutically effective amount of said Polypeptide of the Invention, and/or of a composition comprising the same. The invention also relates to a method for the prevention and/or treatment of at least one disease or disorder that can be prevented and/or treated by administering to a subject suffering from said disease or disorder a Polypeptide of the Invention that is directed against TNF, said method comprising orally administering to said subject a therapeutically effective amount of said Polypeptide of the Invention, and/or of a composition comprising the same. The invention also relates to a method for the prevention and/or treatment of a disease or disorder such as an autoimmune disease (such as e.g. rheumatoid arthritis or Inflammatory Bowel Disease), said method comprising orally administering to said subject a therapeutically effective amount of a Polypeptide of the Invention that is directed against TNF and/or of a composition comprising the same. Accordingly, the present invention also relates to the composition of the invention, wherein the Polypeptide of the Invention is directed against TNF for the prevention and/or treatment of at least one disease or disorder such as an autoimmune disease (such as e.g. rheumatoid arthritis or Inflammatory Bowel Disease).

In another embodiment, the Polypeptide of the Invention is directed against vWF and the invention relates to a method for the prevention and/or treatment of a subject in need of a Polypeptide of the Invention that is directed against vWF, said method comprising orally administering, to said subject, a therapeutically effective amount of said Polypeptide of the Invention, and/or of a composition comprising the same. The invention also relates to a method for the prevention and/or treatment of at least one disease or disorder that can be prevented and/or treated by administering to a subject suffering from said disease or disorder a Polypeptide of the Invention that is directed against vWF, said method comprising orally administering to said subject a therapeutically effective amount of said Polypeptide of the Invention, and/or of a composition comprising the same. The invention also relates to a method for the prevention and/or treatment of a disease or disorder related to platelet-mediated aggregation (such as e.g. the formation of a non-occlusive thrombus, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, thrombotic thrombocytopenic purpura (TTP), transient cerebral ischemic attack, unstable or stable angina pectoris, cerebral infarction, HELLP syndrome, carotid endarterectomy, carotid artery stenosis, critical limb ischaemia, cardioembolism, peripheral vascular disease, restenosis and myocardial infarction), said method comprising orally administering to said subject a therapeutically effective amount of a Polypeptide of the Invention that is directed against vWF and/or of a composition comprising the same. Accordingly, the present invention also relates to the composition of the invention, wherein the Polypeptide of the Invention is directed against vWF for the prevention and/or treatment of at least one disease or disorder related to platelet-mediated aggregation (such as e.g. the formation of a non-occlusive thrombus, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, thrombotic thrombocytopenic purpura (TTP), transient cerebral ischemic attack, unstable or stable angina pectoris, cerebral infarction, HELLP syndrome, carotid endarterectomy, carotid artery stenosis, critical limb ischaemia, cardioembolism, peripheral vascular disease, restenosis and myocardial infarction).

In another embodiment, the Polypeptide of the Invention is directed against IL-6, IL-6R and/or IL-6/IL-6R complex and the invention relates to a method for the prevention and/or treatment of a subject in need of a Polypeptide of the Invention that is directed against IL-6, IL-6R and/or IL-6/IL-6R complex, said method comprising orally administering, to said subject, a therapeutically effective amount of said Polypeptide of the Invention, and/or of a composition comprising the same. The invention also relates to a method for the prevention and/or treatment of at least one disease or disorder that can be prevented and/or treated by administering to a subject suffering from said disease or disorder a Polypeptide of the Invention that is directed against IL-6, IL-6R and/or IL-6/IL-6R complex, said method comprising orally administering to said subject a therapeutically effective amount of said Polypeptide of the Invention, and/or of a composition comprising the same. The invention also relates to a method for the prevention and/or treatment of a disease or disorder associated with IL-6R, IL-6 and/or with the IL-6/IL-6R complex (such as e.g. sepsis, various forms of cancer such as multiple myeloma disease (MM), renal cell carcinoma (RCC), plasma cell leukaemia, lymphoma, B-lymphoproliferative disorder (BLPD) and prostate cancer, bone resorption (osteoporosis), cachexia, psoriasis, mesangial proliferative glomerulonephritis, Kaposi's sarcoma, AIDS-related lymphoma, inflammatory diseases and disorder such as rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus), said method comprising orally administering to said subject a therapeutically effective amount of a Polypeptide of the Invention that is directed against IL-6, IL-6R and/or IL-6/IL-6R complex and/or of a composition comprising the same. Accordingly, the present invention also relates to the composition of the invention, wherein the Polypeptide of the Invention is directed against IL-6, IL-6R and/or IL-6/IL-6R complex for the prevention and/or treatment of at least one disease or disorder associated with IL-6R, IL-6 and/or with the IL-6/IL-6R complex (such as e.g. sepsis, various forms of cancer such as multiple myeloma disease (MM), renal cell carcinoma (RCC), plasma cell leukaemia, lymphoma, B-lymphoproliferative disorder (BLPD) and prostate cancer, bone resorption (osteoporosis), cachexia, psoriasis, mesangial proliferative glomerulonephritis, Kaposi's sarcoma, AIDS-related lymphoma, inflammatory diseases and disorder such, as rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus).

The Polypeptides of the Invention and/or the compositions comprising the same are orally administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific Polypeptide of the Invention to be used and the pharmaceutical formulation or composition to be used, the age, gender, weight, diet, general condition of the subject, and similar factors well known to the clinician.

Generally, the treatment regimen will comprise the oral administration of one or more Nanobodies, polypeptides or proteins of the invention, or of one or more compositions comprising the same, in one or more therapeutically effective amounts or doses. The specific amount(s) or doses to be administered can be determined by the clinician, again based on the factors cited above.

The Nanobodies and polypeptides of the invention may also be used in combination with one or more further therapeutic ingredients (or pharmaceutically active compounds or principles), i.e. as a combined treatment regimen, which may or may not lead to a synergistic effect. Again, the clinician will be able to select such further compounds or principles, as well as a suitable combined treatment regimen, based on the factors cited above and his expert judgement.

When a second active substances or principles is to be used as part of a combined treatment regimen, it can be administered via the same oral route of administration or via a different route of administration, at essentially the same time or at different times (e.g. essentially simultaneously, consecutively, or according to an alternating regime). When the substances or principles are administered to be simultaneously via the same oral route of administration, they may be administered as different formulations or compositions or part of a combined formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

The invention also relates to the use of a Polypeptide of the Invention for the preparation of a composition for the prevention and/or treatment of at least one disease or disorder that can be prevented and/or treated by orally administering to a subject a Polypeptide of the Invention. The invention also relates to the use of a Polypeptide of the Invention directed against a target in the kidney or the bladder for the preparation of a composition for the prevention and/or treatment of at least one disease or disorder that can be prevented and/or treated by orally administering to a subject a Polypeptide of the Invention directed against a target in the kidney or the bladder. The invention also relates to the use of a Polypeptide of the Invention directed against a target in the kidney or the bladder for the preparation of a composition for the prevention and/or treatment of at least one disease or disorder of the kidney or bladder. The invention also relates to the use of a Polypeptide of the Invention directed against a target in the lung for the preparation of a composition for the prevention and/or treatment of at least one disease or disorder that can be prevented and/or treated by orally administering to a subject a Polypeptide of the Invention directed against a target in the lung. The invention also relates to the use of a Polypeptide of the Invention directed against a target in the lung for the preparation of a composition for the prevention and/or treatment of at least one disease or disorder of the lung. The invention also relates to the use of a Polypeptide of the Invention directed against a target on a tumor for the preparation of a composition for the prevention and/or treatment of at least one disease or disorder that can be prevented and/or treated by orally administering to a subject a Polypeptide of the Invention directed against a target on a tumor. The invention also relates to the use of a Polypeptide of the Invention directed against a target on a tumor for the preparation of a composition for the prevention and/or treatment of at least one cancer. The invention also relates to the use of a Polypeptide of the Invention directed against a target in the brain for the preparation of a composition for the prevention and/or treatment of at least one disease or disorder that can be prevented and/or treated by orally administering to a subject a Polypeptide of the Invention directed against a target in the brain. The invention also relates to the use of a Polypeptide of the Invention directed against a target in the brain for the preparation of a composition for the prevention and/or treatment of at least one disease or disorder of the brain (such as neurogenetic diseases, (e.g. Huntington's disease and muscular dystrophy), developmental disorders (e.g. cerebral palsy), degenerative diseases of adult life (e.g. Parkinson's disease and Alzheimer's disease), metabolic diseases (e.g. Gaucher's disease), cerebrovascular diseases (e.g. stroke and vascular dementia), trauma (e.g. spinal cord and head injury), convulsive disorders (e.g. Epilepsy) infectious diseases (e.g. AIDS dementia), obesity, diabetes, anorexia, depression, brain tumors, dementia with Lewy bodies, multi-system atrophy, progressive supranuclear palsy, frontotemporal dementia, vascular dementia or Down's syndrome). The invention also relates to the use of a Polypeptide of the Invention directed against TNF for the preparation of a composition for the prevention and/or treatment of at least one disease or disorder that can be prevented and/or treated by orally administering to a subject a Polypeptide of the Invention directed against TNF. The invention also relates to the use of a Polypeptide of the Invention directed against TNF for the preparation of a composition for the prevention and/or treatment of at least one disease or disorder such as an autoimmune disease (such as e.g. rheumatoid arthritis or Inflammatory Bowel Disease). The invention also relates to the use of a Polypeptide of the Invention directed against vWF for the preparation of a composition for the prevention and/or treatment of at least one disease or disorder that can be prevented and/or treated by orally administering to a subject a Polypeptide of the Invention directed against vWF. The invention also relates to the use of a Polypeptide of the Invention directed against vWF for the preparation of a composition for the prevention and/or treatment of at least one disease or disorder related to platelet-mediated aggregation (such as e.g. the formation of a non-occlusive thrombus, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, thrombotic thrombocytopenic purpura (TTP), transient cerebral ischemic attack, unstable or stable angina pectoris, cerebral infarction, HELLP syndrome, carotid endarterectomy, carotid artery stenosis, critical limb ischaemia, cardioembolism, peripheral vascular disease, restenosis and myocardial infarction). The invention also relates to the use of a Polypeptide of the Invention directed against IL-6, IL-6R and/or IL-6/IL-6R complex for the preparation of a composition for the prevention and/or treatment of at least one disease or disorder that can be prevented and/or treated by orally administering to a subject a Polypeptide of the Invention directed against IL-6, IL-6R and/or IL-6/IL-6R complex. The invention also relates to the use of a Polypeptide of the Invention directed against IL-6, IL-6R and/or IL-6/IL-6R complex for the preparation of a composition for the prevention and/or treatment of at least one disease or disorder associated with IL-6R, IL-6 and/or with the IL-6/IL-6R complex (such as e.g. sepsis, various forms of cancer such as multiple myeloma disease (MM), renal cell carcinoma (RCC), plasma cell leukaemia, lymphoma, B-lymphoproliferative disorder (BLPD) and prostate cancer, bone resorption (osteoporosis), cachexia, psoriasis, mesangial proliferative glomerulonephritis, Kaposi's sarcoma, AIDS-related lymphoma, inflammatory diseases and disorder such as rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus).

As discussed above, oral administration of one or more Polypeptides of the Invention to a subject yields effective delivery of the Polypeptides of the Invention to the blood (and/or another selected physiological compartment, tissue and/or organ such as e.g. the kidney, bladder and/or lung) and/or to the brain to elicit the desired activity or biological response in the subject. In addition to the prophylactic and therapeutic response as discussed above, the Nanobodies, polypeptides and proteins of the invention may also induce other activities and biological responses. In a preferred embodiment, the present invention also provides for the diagnostic use of the Polypeptides of the Invention, e.g. for in situ or in vivo labeling, such as radiolabeling and imaging. The present invention, therefore, also relates to a diagnostic method comprising the step of orally administering the Polypeptides of the Invention and/or a composition comprising the same. In an embodiment of the invention, a diagnostic method is provided comprising the steps of orally administering the Polypeptides of the Invention and/or a composition comprising the same and in situ detecting said Polypeptides of the Invention. Detection may be done by any method known in the art.

The Polypeptides of the Invention can be determined in situ by non-invasive methods including but not limited to SPECT and PET, or imaging methods described by Cortez-Retamozo V. (Nanobodies: single domain antibody fragments as imaging agents and modular building blocks for therapeutics, PhD Dissertation, Vrije Universiteit Brussel, Belgium, June 2004), Arbit et al. (Eur. J. Nucl. Med. 1995; 22: 419-426.), Tamada et al. (Microbiol-Immunol. 1995; 39: 861-871), Wakabayashi et al. (Noshuyo-Byori 1995; 12: 105-110), Huang et al. (Clin. Med. J. 1996; 109: 93-96), Sandrock et al. (Nucl. Med. Commun. 1996; 17: 311-316), and Mariani et al. (Cancer 1997; 15: 2484-2489). These in vivo imaging methods may allow the localization and possibly quantification a certain target, for example, by use of a labeled Polypeptide of the Invention, specifically recognizing said target. In vivo multiphoton microscopy (Bacskai et al., J. Cereb. Blood Flow Metab. 2001; 22: 1035-1041) can be used to image the presence of a certain target with labeled Polypeptides of the Invention specific for the target.

The Polypeptide of the Invention orally administered in the diagnostic methods of the invention may be labeled by an appropriate label. The particular label or detectable group used in the method is not a critical aspect of the invention, so long as it does not significantly interfere with the specific binding of the Polypeptide of the Invention used in the method. The detectable group can be any material having a detectable physical or chemical property. Such detectable labels have been well developed in the field of immunoassays and, in general, almost any label useful in such methods can be applied to the method of the present invention. Thus, a label is any composition detectable by spectroscopic, photochemical, biochemical, immunochemical, electrical, optical, radiological or chemical means. Useful labels in the present invention include but are not limited to magnetic beads (e.g. Dynabeads™), fluorescent dyes (e.g. fluorescein isothiocyanate, Texas red, rhodamine, Cy3, Cy5, Cy5.5, Alexi 647 and derivatives), radiolabels (e.g. ³H, ¹²⁵I, ³⁵S_{,} ¹⁴C, ³²P or ^{99m}Tc), enzymes (e.g. horseradish peroxidase, alkaline phosphatase and others commonly used in an ELISA), and colorimetric labels such as colloidal gold, colored glass or plastic (e.g. polystyrene, polypropylene, latex, etc.) beads.

The label may be coupled directly or indirectly to the Polypeptide of the Invention according to methods well known in the art. As indicated above, a wide variety of labels may be used, with the choice of label depending on the sensitivity required, the ease of conjugation with the compound, stability requirements, the available instrumentation and disposal provisions. Non-radioactive labels are often attached by indirect means. Means for detecting labels are well known in the art. Thus, for example, where the label is a radioactive label, means for detection include a scintillation counter or photographic film as in autoradiography. Where the label is a fluorescent label, it may be detected by exciting the fluorophore with the appropriate wavelength of light and detecting the resulting fluorescence. The fluorescence may be detected visually, by means of a photographic film, by the use of electronic detectors such as charge coupled devices (CCDs) or photomultipliers and the like.

Finally, although the use of the Polypeptides of the Invention (as defined herein, e.g. the Nanobodies and/or constructs comprising said Nanobodies) is much preferred, it will be clear that on the basis of the description herein, the skilled person will also understand that other (single) domain antibodies, as well as polypeptides and proteins comprising such (single) domain antibodies (in which the terms “domain antibody”, “single domain antibody” and “dAb” have their usual meaning in the art) are also encompassed within the scope of the present invention.

The invention will now be further described by means of the following non-limiting experimental part.

### Experimental Part

### Example A

### Nanobodies and Nanobody Constructs Against EpoR and GHR

### Introduction

### General

The erythropoietin receptor (EpoR) and the growth hormone receptor (GHR) belong to the cytokine receptor type I superfamily for which signaling is known to be triggered by ligand-induced receptor homodimerization and mediated by cytoplasmic protein tyrosin kinases of the Jak family (Watowich, 1999; Frank, 2002; Brooks et al, 2007). In the case of the EpoR, upon binding of erythropoietin (Epo), receptor dimerization and activation of the signal transduction pathway lead to erythtroid cell survival, proliferation and differentiation. The GHR dimerization and signaling induced by the growth hormone is the key regulator of postnatal growth and has important actions on metabolism, reproductive, gastrointestinal, cardiovascular, hepto-biliary and renal systems (Brooks et al, 2007). Because of the existence of many clinical situations where the circulating red blood cell levels are reduced provoking anemia, some efforts have been made to develop stable and potent erythropoietin mimetic peptides (EMPs) that activate the receptor by dimerization and thus mimic Epo action. Some bivalent monoclonal antibodies have been described as EpoR agonist since they are capable of forming receptor dimers and stimulate cell proliferation in EpoR-expressing cells, while monovalent Fab fragments fail (Wrighton et al., 1996; Schneider et al., 1997; Skelton et al, 2002; Vadas and Rose, 2007). For the related GH receptor, a variety of agonist monoclonal antibodies have been also reported (Rowlinson et al., 1998).

Structure of EpoR and GHR and Interaction with Natural Ligands

The EpoR and GHR, as other members of the cytokine receptor type I superfamily, are cell surface proteins composed of an NH₂-terminal ligand binding domain, a COOH-terminal cytoplasmic region and a single membrane-spanning domain. Conserved features of the extracellular domain include two pairs of cysteine residues and a ‘WSXWS” motif with characteristic spacing.

While receptor dimerization is a common activation mechanism for this family of cytokine receptors there seem to be small differences between the protein folding pathways and/or three-dimensional structures of individual receptors which dictate their potential to be covalently dimerized by disulfide bridges.

The classical model for activation of GHR is described as the formation of a ligand-receptor complex made up of one GH molecule and two GHR (GH:2 GHR). One of the monomer receptors binds with a strong affinity to site 1 of the GH followed by the weaker site 2 binding to the second receptor (Watowich, 1999; Brooks et al., 2007). Other studies revealed that the receptor can be found as a dimer on the surface of the cell in the absence of GH leading to a paradigm shift whereby most evidences support a model of GH binding to a constitutively homodimerised GHR which causes the recognition of the intracellular domains resulting in the activation of the signal transduction (Brooks et al., 2007; Waters et al, 2006). This current model of signaling also applies to the closely related. EpoR (Watowich, 1999). Early studies of the EpoR/Epo complex suggested a 1:1 stoichiometry, although later studies demonstrated a 2:1 stoichiometry showing two binding sites of Epo for the extracellular domain of the receptor. Interaction of the first site is of high affinity (dissociation constant 1 nM) while the second binding interaction is much weaker (1 uM). There are also some reports that evidence for preformed dimers of EpoR before ligand activation (Livnah et al., 1999; Lu et al., 2006). In that case the binding of Epo changes the orientation of the two receptor subunits, transmitting a conformational change through the transmembrane domains leading to activation of JAK2 kinase and induction of proliferation and survival signals.

### Disease Relation

Reduction of red blood cell levels by a failure in the Epo synthesis provoking anemia is associated to many pathological conditions including chronic renal failure, malignancy or the effects of chemotherapy used to treat cancer, HIV and rheumatoid arthritis (Watowich, 1999. Review. Kontantinopoulos et al., 2007). So Epo is used normally therapeutically administered either by intravenous or subcutaneous injection. However the fact that Epo is large glycoproteins has a negative impact on the cost of the manufacture and on the mode of delivery of this therapeutic agent. Therefore the development of new molecules that can mimic the Epo trough interaction with EpoR is clearly envisaged.

GH has been of significant scientific interest for decades because of its capacity to dramatically change physiological growth parameters. GH has been used for the treatment of adults with GH deficiency and conditions such as Turner's syndrome, Prader-Willie syndrome, intrateurine growth restriction and chronic renal failure (Dattani and Preece, 2004). Mutations in the GHR have been described as the cause of the Laron Syndrome that is characterised by severe postnatal growth retardation (Rosenfeld et al., 1994).

### A—Identification of Agonist Nanobodies Binding EpoR

### Example 1

### Animal Immunizations

Two llamas (215 and 216) will be immunized, according to standard protocols, with 6 boosts of a cocktail 152 containing:

Recombinant mouse EpoR/Fc Chimera (R&D Systems Cat No 1390-ER).

Blood will be collected from these animals 4 and 8 days after boost 6.

### Example 2

### Library Construction

Peripheral blood mononuclear cells will be prepared from blood samples using Ficoll-Hypaque according to the manufacturer's instructions. Next, total RNA will be extracted from these cells and lymph node tissue and used as starting material for RT-PCR to amplify Nanobody encoding gene fragments. These fragments will be cloned into phagemid vector pAX50. Phage will be prepared according to standard methods (see for example the prior art and applications filed by applicant cited herein).

### Example 3

### Selections of Phage Displaying EpoR Binding Nanobodies

Phage libraries 215 and 216 will be used for selections on recombinant mouse EpoR/Fc Chimera (R&D Systems Cat No 1390-ER). rm EpoR/Fc will be immobilized directly or captured by an anti human Fc antibody on Maxisorp 96 well microtiter plates (Nunc) at 5 ug/ml, 0.5 ug/ml and 0 ug/ml (control). To minimize the number of phage binding to the Fc-portion of EpoR/Fc the phage will be pre-incubated with 250 ug/ml human IgG. Following incubation with the phage libraries and extensive washing, bound phage will be totally eluted with trypsin and specifically eluted with Epo. If necessary the eluted phage are amplified and applied in a second round of selection on 5 ug/ml, 0.5 ug/ml, 0.05 ug/ml and 0 ug/ml (control) immobilized EpoR/Fc.

Optionally, the Phage libraries will be pre-incubated with jejunal or gastric fluid prior to selection (analog to Harmsen, 2006, supra) in order to select for protease-resistant Nanobodies. Based on preliminary reports we will chose in one arm a GI fluid concentration that resulted in a decrease in antigen binding capacity in phage ELISA to 10% of an untreated control. In another arm, the Phage libraries will be selected for EpoR binding in the presence of jejunal or gastric fluid (again pre-incubated and not pre-incubated).

Individual colonies of E. coli TG1 infected with the obtained eluted phage pools will be grown and i) induced for new phage production and ii) induced with IPTG for Nanobody expression and extraction (periplasmic extracts) according to standard methods (see for example the prior art and applications filed by applicant cited herein).

### Example 4

### Screening for EpoR Binding Nanobodies

In order to determine binding specificity to EpoR, the clones will be tested in an ELISA binding assay setup, using the monoclonal phage pools. Phage binding to EpoR/Fc Chimera (R&D Systems Cat No 1390-ER) will be tested. Shortly, 0.2 ug/ml receptor will be immobilized on Maxisorp ELISA plates (Nunc) and free binding sites will be blocked using 4% Marvel skimmed milk in PBS. Next, 10 ul of supernatant from the monoclonal phage inductions of the different clones in 100 ul 2% Marvel PBS will be allowed to bind to the immobilized antigen. After incubation and a wash step, phage binding will be revealed using a HRP-conjugated monoclonal-anti-M13 antibody (Gentaur Cat# 27942101). Binding specificity will be determined based on OD values compared to controls having received no phage and to controls where in a similar ELISA binding assay the same monoclonal phage will be tested for binding to 0.2 ug/ml of immobilized human IgG.

### Example 5

### Screening for Nanobodies Competing or Non-Competing (Non-Neutralizing) Epo-EpoR Interaction

Clones tested positive in the EpoR binding assay (including those selected for protease resistancy) will be screened for their ability to block Epo binding to EpoR/Fc. For this, positive binding EpoR phage will be used in an ELISA-based ligand competition setup. 10 ul of supernatant from the monoclonal phage inductions of the different positives clones will be mixed with increasing amounts of EPO and added to 96 well Maxisorp microtiter plates (Nunc) coated with EpoR. After incubation and washing steps, phage binding will be revealed using a HRP-conjugated monoclonal-anti-M13 antibody (Centaur Cat #27942101). Binding specificity will be determined based on OD values compared to controls having received no Epo and/or no phage. The same kind of competition assay could be performed using Nanobody-containing periplasmic extracts (P.E.) instead of phage and detecting with a mouse anti-myc antibody and an anti mouse-HRP antibody.

Clones tested positive in the EpoR binding assay (including clones selected for protease resistancy) will be screened for their ability do not to block EpoR binding to EpoR/Fc. For this, positive binding EpoR phage will be used in an ELISA-based ligand competition setup. 10 ul of supernatant from the monoclonal phage inductions of the different positives clones will be mixed with increasing amounts of EpoR and added to 96 well. Maxisorp microtiter plates (Nunc) coated with EpoR. After incubation, eluted phage containing non-neutralizing Nanobodies will be further analyzed e.g. in BioCore experiments and verified whether indeed they are non-neutralizing Nanobodies. In fact, these non-neutralizing Nanobodies will be used preferably for the construction of agonistic construct comprising e.g. 2 EpoR non-neutralizing Nanobodies. Thus, e.g. various constructs will be generated comprising 2 EpoR non-neutralizing Nanobodies that are identified above, e.g. linked by a 9 Gly linker (see e.g. WO 2007/104529).

### Example 5a

### Testing Screened Non-Competing Nanobodies (Non-Neutralizing) for EpoR Agonism Using MAPPIT (J. Tavernier et al, MAPPIT: a Cytokine Receptor-Based Two-Hybrid Method in Mammalian Cells, Clin. Exp. Allergy 2002; 32: 1397-1404).

Reference is made to the assay described in J. Tavernier et al, supra. In short, a typical screening or test assay comprises the following three successive steps: a) stable transfection of the chimeric “bait” construct, e.g. a construct wherein the extracellular domain of the leptin receptor (LR) is replaced by the murine (see vector pCEL 1f in Tavernier's publication above) or human (see vector pSEL1 same publication) ligand binding extracellular Epo-R gene; b) infection of cells stably expressing the “bait” with above identified bivalent Nanobody constructs; and e) stimulation and selection using puromycin with results in surviving clones that express the agonistically acting bivalent Nanobody construct (confirmation that bivalent Nanobody construct is indeed acting agonistically).

### B—Identification of Agonist Nanobodies Binding GHR

### Example 6

### Animal Immunizations

Two llamas (215 and 216) will be immunized, according to standard protocols, with 6 boosts of a cocktail 152 containing:

Recombinant mouse GHR/Fc Chimera (R&D Systems Cat No 1360-GR).
Blood will be collected from these animals 4 and 8 days after boost 6.

### Example 7

### Library Construction

Peripheral blood mononuclear cells will be prepared from blood samples using Ficoll-Hypaque according to the manufacturer's instructions. Next, total RNA will be extracted from these cells and lymph node tissue and used as starting material for RT-PCR to amplify Nanobody encoding gene fragments. These fragments will be cloned into phagemid vector pAX50. Phage will be prepared according to standard methods (see for example the prior art and applications filed by applicant cited herein).

### Example 8

### Selections of Phage Displaying GHR Binding Nanobodies

Phage libraries 215 and 216 will be used for selections on recombinant mouse GHR/Fc Chimera (R&D Systems Cat No 1360-GR). rm GHR/Fc will be immobilized directly or captured by an anti human Fc antibody on Maxisorp 96 well microtiter plates (Nunc) at 5 ug/ml, 0.5 ug/ml and 0 ug/ml (control). To minimize the number of phage binding to the Fc-portion of GHR/Fc the phage will be pre-incubated with 250 ug/ml human IgG. Following incubation with the phage libraries and extensive washing, bound phage will be totally eluted with trypsin and specifically eluted with GH. If necessary the eluted phage were amplified and applied in a second round of selection on 5 ug/ml, 0.5 ug/ml, 0.05 ug/ml and 0 ug/ml (control) immobilized GHR/Fc. Individual colonies of E. coli TG1 infected with the obtained eluted phage pools will be grown and i) induced for new phage production and ii) induced with IPTG for Nanobody expression and extraction (periplasmic extracts) according to standard methods (see for example the prior art and applications filed by applicant cited herein).

Optionally, the Phage libraries will be pre-incubated with jejunal or gastric fluid prior to selection (analog to Harmsen, 2006, supra) in order to select for protease-resistant Nanobodies. Based on preliminary reports we will chose in one arm a GI fluid concentration that resulted in a decrease in antigen binding capacity in phage ELISA to 10% of an untreated control. In another arm, the Phage libraries will be selected for EpoR binding in the presence of jejunal or gastric fluid (again pre-incubated and not pre-incubated).

### Example 9

### Screening for Nanobodies Competing GH-GHR Interaction or Not Competing GH-GHR

Clones tested positive in the GHR binding assay (including clones selected for protease resistancy) will be screened for their ability to block GH binding to GHR/Fc. For this, positive binding GHR phage will be used in an ELISA-based ligand competition setup. 10 ul of supernatant from the monoclonal phage inductions of the different positives clones will be mixed with increasing amounts of GH and added to 96 well Maxisorp microtiter plates (Nunc) coated with GHR. After incubation and washing steps, phage binding will be revealed using a HRP-conjugated monoclonal-anti-M13 antibody (Gentaur Cat #27942101). Binding specificity will be determined based on OD values compared to controls having received no GH and/or no phage. The same kind of competition assay could be performed using Nanobody-containing periplasmic extracts (P.E.) instead of phage and detecting with a mouse anti-myc antibody and a anti mouse-HRP antibody.

Clones tested positive in the GHR binding assay (including clones selected for protease resistancy) will be screened for their ability not to block GH binding to GHR/Fc. For this, positive binding GHR phage will be used in an ELISA-based ligand competition setup. 10 ul of supernatant from the monoclonal phage inductions of the different positives clones will be mixed with increasing amounts of GH and added to 96 well Maxisorp microtiter plates (Nunc) coated with GHR. After incubation, eluted phage containing non-neutralizing Nanobodies will be further analyzed e.g. in BioCore experiments and verified whether indeed they are non-neutralizing Nanobodies. In fact, these non-neutralizing Nanobodies will be used preferably for the construction of agonistic construct comprising e.g. 2 GHR non-neutralizing Nanobodies. Thus, e.g. various constructs will be generated comprising 2 GHR non-neutralizing Nanobodies that are identified above, e.g. linked by a 9 Gly linker (see e.g. WO 2007/104529).

### Example 9a

### Testing Screened Non-Competing Nanobodies (Non-Neutralizing) for Identifying GHR Agonism Using MAPPIT (J. Tavernier et al., MAPPIT: a Cytokine Receptor-Based Two-Hybrid Method in Mammalian Cells, Clin. Exp. Allergy 2002; 32: 1397-1404).

Reference is made to the assay described in J. Tavernier et al, supra. In short, a typical screening or test assay comprises the following three successive steps: a) stable transfection of the chimeric “bait” construct, e.g. a construct wherein the extracellular domain of the leptin receptor (LR) is replaced by the murine (see vector pCEL if in Tavernier's publication above) or human (see vector pSEL1 same publication) ligand binding extracellular Epo-R gene; b) infection of cells stably expressing the “bait” with above identified bivalent Nanobody constructs; and c) stimulation and selection using puromycin with results in surviving clones that express the agonistically acting bivalent Nanobody construct (confirmation that bivalent Nanobody construct is indeed acting agonistically).

### Review Articles on GHR

### Frank S J.

Receptor dimerization in GH and Erythropoietin action—It takes two to tango, but how? Endocrinology (2002). 143: 2-10.

### Watowich S S.

Activation of erythropoietin signaling by receptor dimerization. The International Journal of Biochemistry & Cell biology (1999). 31: 1075-1088.

### Brooks A J, Wooh J W, Tunny K A and Waters M J.

Growth hormone receptor; mechanism of action. The International Journal of Biochemistry & Cell biology (2007). Doi: 10.1016/j. biocel. Jul. 8, 2007.

### Flores-Morales A, Greenhalgh C J, Norstedt G and Rico-Bautista E.

Negative regulation of growth hormone receptor signaling. Molecular Endocrinology (2006). 20: 241-253.

### Waters M J, Hoang H N, Pelekanos R A and Brown R J.

New insights into growth hormone action. Journal of Molecular Endocrinology (2006). 36: 1-7.

### C—Identification of Nanobodies Binding to Human FcRn

### Introduction

### General

The major histocompatibility complex class I-related receptor FcRn was first identified as the receptor that transports maternal IgGs from mother to young via the neonatal intestine. However recent data have indicated that the neonatal receptor is also responsible for rescuing IgG and albumin from degradation and therefore prolong their half-lives (Andersen et al., 2006; Anderson et al, 2006; Ghetie and Ward, 2000; Kim et al., 2006; Lencer and Blumberg, 2005; Ober et al., 2004a; Ober et al., 2004b). FcRn is expressed inside endothelial cells that line blood vessel, mainly in early/recycling endosomes, where IgG and albumin can be internalized by fluid phase endocytosis. To a minor extend FcRn is also express in the cell surface. IgG and albumin bind independently to FcRn in a pH-dependent manner, with binding at pH 6.0 but not at pH 7.4. The acidic environment of the endosomes facilitates the interaction. Bound IgG and albumin are recycled back to the surface and released from the cell, while unbound ligands are shuttled downstream to lysosomal degradation (Ghetie and Ward, 2000).

The role of FcRn as an IgG transporter opens the opportunity to generate new therapeutics for modulation of IgG levels, as it is desired in the case of autoimmune diseases. Because the transport and protection of IgG are dependent on its Fc-domain, it can be proposed that small molecules or peptides with therapeutic activities could be fused to Fc fragments and therefore delivered across the epithelium and have long circulating long lives. Moreover, the fact that FcRn is expressed on many epithelial surfaces in adult humans including the lungs (Spiekermann et al., 2002; Bitonti and Dumont, 2006), suggests that FcRn transport pathway could be used as a delivery system of therapeutic agents by non-invasive means (i.e. aerosols administered into the lungs using normal breathing maneuvers).

### Structure

FcRn comprises a heterodimer of beta2-microglobulin and a 45 to 53 KDa protein. All three extracellular and membrane domains of FcRn share homology with the corresponding regions of major histocompatibility complex (MHC) class I molecules, with much less homology between the cytoplasmic domains. The X-ray crystallographic structure of the extracellular domains of FcRn confirmed that it is structurally similar to MHC class I molecules (Ghetie and Ward, 2000).

The FcRn-IgG interaction depends on conserved histidine residues in the IgG-Fc part that interact with negatively charged residues in the beta-2 domain of the hFcRn heavy chain. Recent studies showed that conserved H166 in the hFcRn heavy chain, directly opposite to the IgG binding site, is a key player in the FcRn-albumin interaction (Andersen et al., 2006).

### Disease Relation

The fact that FcRn regulates IgG homeostasis, modulation of FcRn function and/or expression might be an effective approach for the treatment of autoimmune diseases. It has been suggested that deregulation of FcRn expression may be involved in situations in which hypercatabolism is observed, such as after burns and in myotonic dystrophy. It is also possible that some types of IgG deficiencies such as familial idiophatic hypercatabolism may be caused by abnormalities in FcRn expression or function (Ghetie and Ward, 2000).

Identification of Nanobodies Binding hFcRn:

### Members:

| Families of binders (binders with same CDR3): | members: |
|---|---|
| I | 215-C3, 215-E2, 215-B5 |
| II | 215-B3 |
| III | 216-E11 |
| IV | 215-A4 |

### Sequences:

SEQ ID NO: 1 - LG215-C3
EVQLVESGGGLVQPGGSLRLSCAASGFTFSDHAMSWYRQAPGKGLEWVSA
INTAGIITNTADSVKGRFTMSRDNAKNTLYLQMNSLKPEDTAKYYCARNR
DINNSRPPESQGTQVTVSS
SEQ ID NO: 2 - LG215-E2
EVQLVESGGGLVQPGOSLRLSCAASGFTFSDYAMSWVRQAPGKGLEWVSA
INTAGIITNYADSVKGRFTMSRDNAKNTLYLQMNSLKPEDTAKYYCARNR
DINNSRPPESQGTQVTVSS
SEQ ID NO: 3 - LG215-B5
EVQLVESGGGLVQPGGSLRLSCAASGFTFSDYAMSWVRQAPGKGLEWVSA
INTAGILTNYADSVKGRFTMSRDNAKNTLYLQMNSLKPEDTGKYYCARNR
DINNSRPPESQGTQVTVSS
SEQ ID NO: 4 - LG215-B3
EVQLVESGGGSVQPGGSLRLSCLASGRTFSTYFMGWFRQAPGKEREFVTA
ISASGGTTYYADSVKGRFTISRDNAKNTVYLQMNSLKPEDTAVYYCAANN
RYYSDYMKLWPYDYWGQGTQVTVSS
SEQ ID NO: 5 - LG216-E11
EVQLVESGGGLVQPGGSLRLSCVASAYIFNINHMGWYRQAPGKRREVVAA
VSSAGNTYYADSVKGRFTISRDDAKNTVYLQMNNLKPEDTAVYYCNRLAP
TGGTWPDYWGQGTQVTVSS
SEQ ID NO: 6 - LG215-A4
EVQLVKSGGGLVQAGGSLRLSCAASGRSFGYYAMAWFRQAPGKEREFVAA
FTWNGVSTTTYYADSVKGRFTISRDNAKNTGYLQMNSVRPEDTAVYYCAA
RSGFYSRQKSEYPYWGQGTQVTVSS

### Sequences Alignment:

LG215-C3
evqlvesggglvqpggslrlscaasgftfsDHAMSwvrqapgkglewvs.
.AINTAGIIT
LG215-E2
evqlvesggglvqpggslriscaasgfTfsDYAMSwvrqapgkglewvs.
.AINTAGIIT
LG215-B5
evqlvesggglvqpggslrlscaasgftfsDYAMSwvrqapgkglewvs.
.AINTAGILT
LG215-B3
evqlvesgggsvqpggslrlsclasgrtfsTYFMGwfrqapgkerefvt.
.AISASGGTT
LG216-E11
evqlvesggglvqpggslrlscvasayifnlNHMGwyrqapgkrrevva.
..AVSSAGNT
LG215-A4
evqlvksggglvqaggslrlscaasgrsfgYYAMAwfrqapgkerefvaA
FTWNGVSTTT
LG215-C3
NYADSVKGrftmsrdnakntlylqmnslkpedtakyycar.NRDINNSRP
P.....esqg
LG215-E2
NYADSVkGrftmsrdnakntlylqmnslkpedtakyycar.NRDINNSRP
P.....esqg
LG215-B5
NYADSVkGrftmsrdnakntlylqmnslkpedtgkyycar.NRDINNSRP
P.....esqg
LG215-B3
YYADSVKGrftisrdnakntvylqmnslkpedtavyycaaNNRYYSDYMK
LWPYDYwgqg
LG216-E11
YYADSVKGrftisrddakntvylqmnnlkpedtavyycnr....LAPTGG
TWPDY.wgqg
LG215-A4
YYADSVKGrftisrdnakntgylqmnsvrpedtavyycaaRSGFYSRQKS
EYPY..wgqg
LG215-C3
tqvtvss
LG215-E2
tqvtvss
LG215-B5
tqvtvss
LG215-B3
tqvtvss
LG216-E11
tqvtvss
LG215-A4
tqvtvss

### Example 10

### Animal Immunizations

Llama 153 was immunized, according to standard protocols, with 6 boosts of a cocktail 112 containing hFcRn HC (only the human FcRn heavy chain). hFcRn HC and shFcRn mutmix were kindly provided by Inger Sandlie (University of Oslo, Norway).

Llama 154 was immunized, according to standard protocols, with 3 boosts of a cocktail 116 containing shFcRn mutmix (intact soluble FcRn, heavy chain and beta2-microglobulin).

For animal 153, blood was collected 4 and 7 days after boost 6. In addition, approximately 1 g of lymph node was collected from this animal 4 days after boost 6

For animal 154, blood was collected 22 days after boost 3.

### Example 11

### Library Construction

Peripheral blood mononuclear cells were prepared from blood samples using Ficoll-Hypaque according to the manufacturer's instructions. Next, total RNA was extracted from these cells and lymph node tissue and used as starting material for RT-PCR to amplify Nanobody encoding gene fragments. These fragments were cloned into phagemid vector pAX50. Phage was prepared according to standard methods (see for example the prior art and applications filed by applicant cited herein).

### Example 12

### Selections of Phage Displaying hFcRn Binding Nanobodies

Phage library 153 was used for selection at pH 5 on hFcRn heavy chain (hFcRn HC) while phage library 154 was used for selection at pH 5 on shFcRn (heavy chain and beta2-microglobulin). Both hFcRn proteins were immobilized directly on Maxisorp 96 well microtiter plates (Nunc) at 5 ug/ml, 0.5 ug/ml and 0 ug/ml (control) in PBS at pH 7.4. After 2 hours blocking with 4% Marvel PBS the plates were washed several times with PCA buffer/Tween pH 5.1 (10 mM Sodium citrate+10 mM Sodium phosphate+10 mM Sodium acetate+115 mM NaCl/Tween pH 5.1) To minimize the number of phage binding to the albumin binding site of the FcRn protein the phage was pre-incubated with 250 ug/ml human serum albumin in 2% Marvel PCA buffer pH 5.1. Following incubation with the phage libraries and extensive washing with pH 5.1 buffer, bound phage was eluted with trypsin. The eluted phage were amplified and applied in a second round of pH 5 selection on 5 ug/ml, 0.5 ug/ml and 0 ug/ml (control) immobilized hFcRn proteins. To minimize the number of phage binding to the albumin binding site of the FcRn protein the phage was pre-incubated with 250 ug/ml human serum albumin in 2% Marvel PCA buffer pH 5.1. Individual colonies obtained from the eluted phage pools were grown and i) induced for new phage production and ii) induced with IPTG for Nanobody expression and extraction (periplasmic extracts) according to standard methods (see for example the prior art and applications filed by applicant cited herein).

### Example 13

### Screening for hFcRn Binding Nanobodies

In order to determine binding specificity to hFcRn, the clones were tested in an ELISA binding assay setup, using the monoclonal phage pools. Phage binding to hFcRn HC was tested. Shortly, 0.5 ug/ml hFcRn HC was immobilized on Maxisorp ELISA plates (Nunc) and free binding sites were blocked using 4% Marvel skimmed milk in PBS. After washing with PCA ph 5.1 buffer, 10 ul of supernatant from the monoclonal phage inductions of the different clones in 100 ul 2% Marvel PCA pH 5.1 were allowed to bind to the immobilized antigen. After incubation and several wash steps with pH 5.1 buffer, phage binding was revealed using a HRP-conjugated monoclonal-anti-M13 antibody (Gentaur Cat# 279421.01) in 1% Marvel PCA pH 5.1.

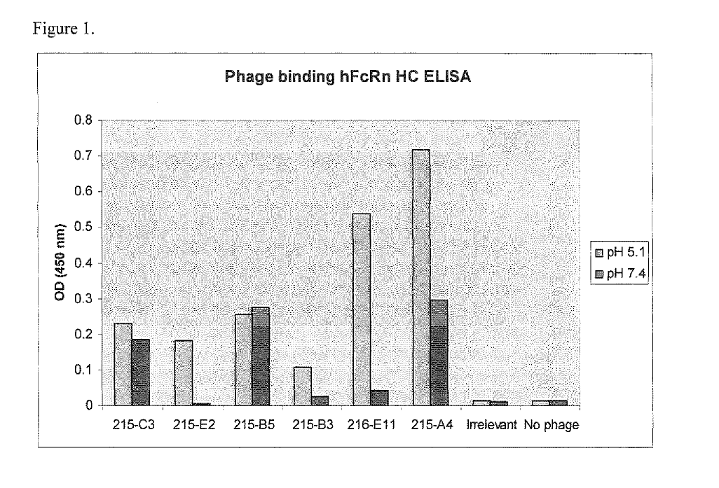

The same ELISA assay was performed at neutral pH by using PCA pH 7.4 buffer. Binding specificity was determined based on OD values compared to controls wells having received an irrelevant phage or no phage. FIG. 1 shows a selection of clones binding to hFcRn HC at pH 5.1 and pH 7.4

### Review Articles on FcRn

### Anderson C L, Chaudhury C, Kim J, Bronson C L, Wani M A and Mohanty S.

Perspective-FcRn transport albumin: relevance to immunology and medicine. TRENDS in Immunology (2006). 27: 343-348

### Ghetie V and Ward S E.

Multiple roles for the major histocompatibility complex class I-related receptor FcRn. Annu. Rev. Immunol. (2000) 18: 739-766

### Lencer W I and Blumberg R S.

A passionate kiss, then run: exocytosis and recycling of IgG by FcRn. TRENDS in cell biology (2005). 15: 5-9.

### D—Identification of pIgR Binding Nanobodies and IgA Competitors

### Example 14

### Immunizations

Two llamas (097 and 098) were immunized with 6 boosts of R&D Systems Cat #2717-PG, which is the ectodomain of human pIgR, according to standard protocols. Blood was collected from these animals after 7 days after boost 6 and 10 days after boost 6.

### Example 15

### Library Construction

Peripheral blood mononuclear cells were prepared from blood samples using Ficoll-Hypaque according to the manufacturer's instructions. Next, total RNA extracted was extracted from these cells and used as starting material for RT-PCR to amplify Nanobody encoding gene fragments. These fragments were cloned into phagemid vector pAX50. Phage was prepared according to standard methods (see for example the prior art and applications filed by applicant cited herein) and stored after filter sterilization at 4° C. for further use.

### Example 16

### Selections

Phage libraries from llama's 097 and 098 were used for selections for two rounds on ectodomain of pIgR (R&D Systems Cat #2717-PG). pIgR was immobilized directly on Nunc Maxisorp ELISA plates at 5 microg/ml or 1 ug/ml and 0 ug/ml (low control) for the first round of selection and 5 microg/ml or 0.5 ug/ml and 0 ug/ml (low control) for the second round of selection. Binding phages were retrieved from both first and second selection rounds using trypsin elution, IgA specific elution and BSA specific elution (neg. control).

Specific elution was performed by incubating the wells with 150 ug/ml IgA for 1 hour, thereby replacing Nanobodies binding on the IgA binding spot of pIgR.

For the second round of selection phages from the output of the first round of selection eluted with IgA were used.

Output of both R1 and R2 selections were analyzed for enrichment factor (# phage present in eluate relative to control). Based on these parameters the best selections were chosen for further analysis. Individual colonies were picked and grown in 96 deep well plates (1 ml volume) and induced by adding IPTG for Nanobody expression. Periplasmic extracts (volume: ˜80 ul) were prepared according to standard methods (see for example the prior art published and applications filed by applicant).

### Example 17

### Screening

In order to determine binding specificity to pIgR, the clones were tested in an ELISA binding assay setup.

In short, 5 ug/ml pIgR ectodomain was immobilized on Maxisorp microtiter plates (Nunc) and free binding sites were blocked using 4% Marvel in PBS. Next, 10 ul of periplasmic extract containing Nanobody of the different clones in 100 ul 2% Marvel PBST were allowed to bind to the immobilized antigen. After incubation and a wash step, Nanobody binding was revealed using a mouse-anti-myc secondary antibody, which was after a wash step detected with a HRP-conjugated donkey-anti-mouse antibody. Binding specificity was determined based on OD values compared to controls having received no Nanobody (low control). Overall more than 70% of the selected clones were able to bind to pIgR with some specificity (signal more than 2× above background).

### Example 18

### Screening for Competition

In order to determine IgA competition efficiency of pIgR binding Nanobodies the positive clones of the binding assay were tested in an ELISA competition assay setup. In short, 5 ug/ml pIgR ectodomain was immobilized on Maxisorp microtiter plates (Nunc) and free binding sites were blocked using 4% Marvel in PBS. Next, 1 ug/ml of IgA was preincubated with 10 ul of periplasmic extract containing Nanobody of the different clones and a control with only IgA (high control). The IgA was allowed to bind to the immobilized receptor with or without Nanobody. After incubation and a wash step, IgA binding was revealed using a rabbit-anti-IgA secondary antibody, which was after a wash step detected with a HRP-conjugated donkey-anti-rabbit antibody. Binding specificity was determined based on OD values compared to controls having received no Nanobody (high control).

### Example 19

### Determining Competition Efficiency by Titration of Purified Nanobody

In order to determine IgA competition efficiency of IgA competitive Nanobodies clones of the binding assay were tested in an ELISA competition assay setup.

In short, 5 ug/ml pIgR ectodomain was immobilized on Maxisorp microtiter plates (Nunc) and free binding sites were blocked using 4% Marvel in PBS. Next, 1 ug/ml of IgA was preincubated with a dilution series of purified Nanobody and a control with only IgA (high control). The IgA was allowed to bind to the immobilized receptor with or without Nanobody. After incubation and a wash step, IgA binding was revealed using a rabbit-anti-IgA secondary antibody (Serotec cat #AHP525H), which was after a wash step detected with a HRP-conjugated donkey-anti-rabbit antibody. Binding specificity was determined based on OD values compared to controls having received no Nanobody (high control) and two Nanobodies that can bind to pIgR but do not compete for IgA binding.

The results confirm that clones 1D2, 1D7, 1E7, 4B11 and 4D9 have a antagonistic effect on IgA binding to pIgR. 1D2 and 4B11 are inferior in this to the other clones.

### Example 20

### Nanobody Binding on Living Cells Overexpressing pIgR

In order to determine binding specificity to pIgR in cells Nanobody 4B11 was tested in an immunofluorescence setup (adapted from Klapisz 2002).

In short, MDCK cells overexpressing human pIgR were grown on glass cover slips and free binding sites were blocked using precooled 4% Marvel in PBS at 4C. Next, 3 uM of purified Nanobody in 50 ul 2% Marvel PBS was allowed to bind to the cells at 4C. After incubation and a wash step, cells were fixed using 4% paraformaldehyde. Nanobody binding was revealed using a mouse-anti-myc secondary antibody, which was after a wash step detected with a Cy2-conjugated donkey-anti-mouse antibody. For reference nuclei were stained using DAPI. Fluorescence signal was detected under an epifluorescence microscope (Leica) attached to a cooled CCD camera (Micromax, Princeton Instruments). The pictures were taken using Metamorph and the final figures were obtained using the NIH Image and Adobe Photoshop programs. Pictures show that Nanobody 4B11, 1D2 and 1E7 can bind to human pIgR in a cellular environment.

### Example 21

### Nanobodies are Able to Bind the hpIgR in its Native Form

In order to check whether Nanobodies are able to bind the hpIgR in its native form, an immunoprecipitation experiment was performed nanobodies, containing a His-tag, were allowed to bind to hpIgR in cell lysates and fished out with talon beads. The hpIgR was detected on blot with a-hSC and DAG-PO. As a control the VHHs bound to the beads were detected with a-myc and DAM-PO. The result of this immunoprecipitation experiment clearly shows that the VHHs: 1D2, 4B11, 4B7 and 1D7 are able to bind to hpIgR in cell lysates. The receptor could be detected in the four lanes containing lysates with the hpIgR binding VHHs. Empty talon beads and nanobodies directed against the EGF receptor were not able to detect the receptor and the receptor was also not detected in lysates of untransfected MDCK cells. The lysate control shows that the nanobodies are able to enrich for this receptor out of cell lysate. The binding of the Nanobodies to the talon beads was checked and this shows that indeed all lanes contained beads with bound VHHs, except for the empty beads and the VHHs were also not present in the cell lysate.

### Example 22

### Transcytosis Capacity of the Nanobodies

In order to check the transcytosis capacity of the Nanobodies, transwell experiments were performed. Two different nanobodies, namely 1D2 (IgA-competing) and 4B7 (non-competing) were recloned into a phagemid vector and monoclonal phages were produced. We wanted to show that the phages were able to transcytose across MDCK cells expressing pIgR from the basolateral to the apical side.

The transcytosis assay is performed with fully polarized MDCK cells, seeded on 1 cm², 0.4 μm collagen-coated PTFE transwell filters (Costar).

Lucifer Yellow (LY) was added to the basolateral chamber one hour before the experiment as a control for monolayer integrity and aspecific transport. The concentration of LY in the apical chamber was determined by measuring fluorescence. When the apical LY samples showed no leakage or a-specific transport transcytosis experiments are performed.

10⁶ phages were added to the basolateral chamber of the Transwell-system and allowed to transcytose for 5 hours. Samples were taken from the apical chamber and the total, amount of transcytosed phages was determined.

Monoclonal phages 1D2 and 4B7 are able to transcytose across the monolayer of MDCK cells bearing the hpIgR, whereas they can not cross the MDCK cells without hpIgR. Also an irrelevant phage expressing GST-binding nanobody did not transcytose across transfected or untransfected cells.

This showed that phages 1D2 and 4B7 are transcytosed over MDCK cells by hpIgR in a transwell assay from basolateral to apical.

TABLE D-1
1D7 SEQ ID NO: 7
EVQLVESGGGLVQSGGSLRLSOAASGRAFNYYAMGWFRQAPGKERELVAV
INWSGGTTSYADSVKGRFTISRSNAKNTVYLQMSSLKPEDTAVYYCAADS
IYRTSKDYNYWGQGTQVTVSS
1 E7 SEQ ID NO: 8
EVQLVESGGGLVQAGGSLRLSCAASGRTFSNYVMGWFRQAPGKEREFVAA
ISWSGVTTYHYSADSVKGRFTTSRDNDRNTAHLQMNSLKPEDTAVYYCAA
RGRTGSDPRKGDDYDYWGQGTQVTVSS
1_d2 SEQ ID NO: 9
EVQLVESGGGLVQPGGSLRLSCAASGFTLDYYAIGWFRRAPGKEREGVSC
TSSSDGSTYYADSVKGRFTISRDNAKNTVYLQMNSLKPEDTAVYYCATTF
GDACTVVAGIPDQYDFGSWGQGTQVTVSS
4_b11 SEQ ID NO: 10
EVQLVESGGGLVQPGGSLRLSCAASGFTLDYYAIGWFRQAPGKEREGVSC
ISSSDMSDGITYYADSVKGRETSSRDNVKNTVYLQMNSLKPEDTAVYYCA
TTFGDACTVVAGIPDQADFDSWGQSTQVTVSS
4_d9 SEQ ID NO: 11
EVQLVESGGGLVQPGGSLRLSCAASGFTLDYYDIGWFRQAPGKEREGVSC
TSSSDGSTYYADSVKGRFTISRDNAKNTVYLQMNSLKPEDTAVYYCATTF
GDACTVVAGIPOQYDFGSWGQGTQVTVSS
4_a7 SEQ ID NO: 12
EVQLVESGGGLVQAGGSLRLSCEDSGRTFGDYIMGWFRQAPGKERDFVAA
ISWTGDSTYYKYYSDSAKGRFTASRDNAKNTAYLQMNGLKPEDTAVYYCA
ARTFRIDYDPRTASTYNYWGQGIQVTVSS
4_h1 SEQ ID NO: 13
EVQLVKSGGGLVQAGDSLRLSCAASGRTSSSVTMGWFRQAPGKERDFVAA
ISWSGGSTYYADSVKGRFTISRDNAKNTVYLQMNSLKPEDTAVYYCAAVV
PSDPIIYYTDYVDYDYWGQGTQVTVSS
4_b7 SEQ ID NO: 14
EVQLVESGGGLVQAGGSLRLSCAASGFTFDDYAIGWFRQAPGKEREGVSC
ISSRDGITYYADSVKGRFTISSDNAKNTVYLQMNSLKPEDTAVYYCAADL
VGSFPCPVAAYDYWGQGTQVTVSS
4_g6 SEQ ID NO: 15
EVQLVESGGGLVQAGGSLRLSCAASGFTFDDYAIGWFRQAPGKEREGVSC
ISSRDGTTYYADSVKGRFTISSDNAKNTVYLQMNSLKPEDTAVYYCAADL
VGSFPCPVAAYDYWGQSTQVTVSS
4_e111 SEQ ID NO: 16
EVQLVESGGGLVQAGGSLRLSCAASGFTFDDYAIGWFRQAPGKEREGVSC
INNSDGSTYYADSVKGRFTISSDNAKNTVYLQMNSLKPEDTAVYYCAADF
VGGSYCLFPTYNYWGQCTQVTVSS
1_g10 SEQ ID NO: 17
EVQLVESGGGLVQAGDSLRLSCAASGRTFSTYAMAWFRQAPGKDREFVAA
ISWSSDMTYYLDSVKGRFTISRDNAKNTVFLQMNSLKPEDTAVYYCASGA
YYAGSSTSPYNYWGQGTQVTVSS
2_f3 SEQ ID NO: 18
EVQLVESGGGLVQAGGSLRLSCAASGRTFTIYTMGWFRQAPGKAREFISA
LRWSGGSTYTFYADSVKGRFTISRDNAKNTLYLQMNSLKSEDTAIYYCGM
VDPRAPYMRPDSTDSYAYWGQGTQVTVSS
2_d2 SEQ ID NO: 19
EVQLVESGGGQVQAGGSLRLSCVASERTFSYYDLAARAWFRQAPGKEREL
VSASTWNGGYTYYVDSVKGRFTVSTDDAGDTMYLQMNSLEPEDTAVYYCA
ARRAYSSDLHDYRTFDYWGQGTQVTVSS
1B7 SEQ ID NO: 20
EVQLVESGGGLVQAGGSLRLSCAASGFTFDDYAIGWFRQAPGKEREWVSC
ISRSDGSTYYADSVKGRFTISSDNAKNTVYLQMNSLKPEDTAVYHCAADA
IGSFPCPAGVYDYWGQGTQVTVSS
1C2 SEQ ID NO: 21
EVQLVESGGGLAQPGGSLRLSCAASGFAFSSYWMYWVRQAPGKGLEWVSA
ISTGGGGTYYADSVKGRFTISRDNAKNTLYLQMNSLKPEDTARYYCARDE
APTFDYSGNYAYTGSDPNDYWGQGTQVTVSS
1C7 SEQ ID NO: 22
EVQLVESGGGLVQPGGSLRLSCAASGFAFSRYGMYWVRQAPGKGLEWVST
INSGGDYIIYADSVKGRFTISRDNAKNTLYLQMNSLKPEDTAVYYCAEGY
RGSEWPPPAFTLQRADFASWGQGTQVTVSS
1 E2 SEQ ID NO: 23
EVQLVESGGGLVQAGGSLRLSCVASGFILREYNMGWFRQAPGKEREIVAA
IAWTGTNSYYVDSVKGRFTISRDDTKNTVYLQMNSLNPEDTGVYHCAAEG
YVSNFPRSSADEYDYWGQGTQVTVSS
4C11 SEQ ID NO: 24
EVQLVESGGGLVQPGGSLRLSCAASGFTFSSYAMTWVRQGLGKCLEWVSI
ISGNGGSTSYADSVKGRFTISRDNAKNTLYLQMNSLKPEDTAVYYCAKWD
GLGTLPGSQGTQVTVSS
4 E9 SEQ ID NO: 25
EVQLVESGGGLVQAGGSLRLSCAASGSILSTNDMGWYRQAPGNQRELVAR
ISRGSSTIYTESVKGRFTISRDNAKNTVYLQMNSLKPEDTAVYYCNADML
PSDLSHGYYYRDYWGQGTQVTVSS
4G11 SEQ ID NO: 26
EVQLVESGGGLVQAGGSLRLSCAASGFTFDDYAIGWFRQAPGKEREGVSC
ISSRDGMTYYADSVKGRFTISSDNARNTVYLQMNSLKPEDTAVYYCAADL
VGSFPCPVAAYDYWGQGTQVTVSS
48D6 SEQ ID NO: 27
EVQLVESGGGLVQAGDSLTLSCVASGRTFSAYGMGWFRQAPGKEREFVAS
INWGGGNTYYANSVKDRFAISKDHAKNTVYLQMNSLKPEDTALYYCAAVS
SNTEIFDTWGQGIQVTVSS

### E—Identification of Conditional Serum Albumin Specific Nanobodies

### Example 23

### Immunization

After approval of the Ethical Committee of the Faculty of Veterinary Medicine (University Ghent, Belgium), 2 llamas (117, 118) were alternately immunized with 6 intramuscular injections at weekly interval with human serum albumin and a mixture of mouse serum albumin, cynomolgus serum albumin and baboon serum albumin, according to standard protocols.

### Example 24

### Library Construction

When an appropriate immune response was induced in llama, four days after the last antigen injection, a 150 ml blood sample is collected and peripheral blood lymphocytes (PBLs) were purified by a density gradient centrifugation on Ficoll-Paque™ according to the manufacturer's instructions. Next, total RNA was extracted from these cells and used as starting material for RT-PCR to amplify Nanobody encoding gene fragments. These fragments were cloned into phagemid vector pAX50. Phage is prepared according to standard methods (see for example the prior art and applications filed by applicant cited herein) and stored at 4° C. for further use.

### Example 25

### Selecting Repertoires for Binding to Serum Albumin

In a first selection, human serum albumin (Sigma A-8763) was coated onto Maxisorp 96-well plates (Nunc, Wiesbaden, Germany) at 100 μg/ml overnight (ON) at room temperature (RT). Plates were blocked with 4% Marvel in PBS for 2 h at RT. After 3 washes with PBST, phages were added in 4% Marvel/PBS and incubated for 1 h at RT. Following extensive washing, bound phage was eluted with 0.1 M triethanolamine (TEA) and neutralized with 1M Tris-HCl pH 7.5.

### Example 26

### Selecting Repertoires for Conditional Binding to Serum Albumin

To enrich for conditional binders, said binders with a pH sensitive interaction, phage libraries were incubated with antigen at physiological pH and eluted at acidic pH as follows.

In a first selection, human serum albumin (Sigma A-8763) was coated onto Maxisorp 96-well plates (Nunc, Wiesbaden, Germany) at 100 μg/ml overnight (ON) at room temperature (RT). Plates are blocked with 4% Marvel in PBS pH 7.3 for 2 h at RT. After 5 washes with PBS/0.05% Tween20 (PBST) pH 7.3, phages were added in 2% Marvel/PBS pH 7.3 and incubated for 2 h at RT. Unbound phages were removed by 10 washes with PBST pH7.3, followed by 2 washes with PBS pH5.8. Bound phage was eluted with PBS pH5.8 for 30 min at RT and neutralized with 1M Tris-HCl pH. 7.5.

In a second selection, phage libraries were incubated for 2 h at RT with human serum albumin in 2% Marvell/CPA buffer (10 mM sodium citrate+10 mM sodium phosphate+10 mM sodium acetate+115 mM NaCl) adjusted to pH 7.3. Unbound phages were removed by 10 washes with CPA/0.05% Tween20 (CPAT) pH7.3, followed by 2 washes with CPAT pH5.8. Bound phage was eluted with CPA pH5.8 for 30 min at RT and neutralized with 1M Tris-HCl pH 7.

In a third selection strategy, phage libraries were incubated for 2 h at RT with human serum albumin in 2% Marvell/CPA pH 5.8. Unbound phages are removed by 10 washes with CPAT pH 5.8, followed by 2 washes with CPA pH 7.3. Bound phage was eluted with 1 mg/ml trypsin/CPA pH 7.3 for 30 min at RT.

In a fourth selection strategy, phage libraries were incubated for 2 h at RT with human serum albumin in 2% Marvell/PBS pH5.8. Unbound phages are removed by 10 washes with PBST pH5.8, followed by 2 washes with PBSpH 7.3. Bound phage was eluted with 1 mg/ml trypsin/CPA pH 7.3 for 30 min at RT.

In all selections, enrichment was observed. The output from each selection was re-cloned as a pool into the expression vector pAX51. Colonies were picked and grown in 96 deep-well plates (1 ml volume) and induced by adding IPTG for Nanobody expression. Periplasmic extracts (volume: ˜80 μl) were prepared according to standard methods (see for example the prior art and applications filed by applicant).

### Example 27

### Library Evaluation by ELISA

Periplasmic extracts of individual Nanobodies were screened for albumin specificity by ELISA on solid phase coated human serum albumin. Detection of Nanobody fragments bound to immobilized human serum albumin was carried out using a biotinylated mouse anti-his antibody (Serotec MCA1396B) detected with Streptavidin-HRP (DakoCytomation #P0397). The signal was developed by adding TMB substrate solution (Pierce 34021) and detected at a wavelength of 450 nm. A high hit rate of positive clones can already be obtained after panning round 1.

### Example 28

### Selection for Conditional or pH-Sensitive Binding of Nanobodies to Albumin by ELISA

To enrich for conditional binders, said binders with a pH sensitive interaction, phage libraries may be incubated with antigen at physiological pH and eluted at acidic pH as follows.

In a first selection strategy, human serum albumin (Sigma A-8763) is coated onto Maxisorp 96-well plates (Nunc, Wiesbaden, Germany) at 100 μg/ml overnight (ON) at room temperature (RT). Plates are blocked with 4% Marvel in PBS pH 7.3 for 2 h at RT. After 5 washes with PBS/0.05% Tween20 (PBST) pH 7.3, phages were added in 2% Marvel/PBS pH 7.3 and incubated for 2 h at RT. Unbound phages were removed by 10 washes with PBST pH7.3, followed by 2 washes with PBS pH5.8. Bound phage was eluted with PBS pH5.8 for 30 min at RT and neutralized with 1M Tris-HCl pH 7.5.

In a second selection strategy, phage libraries were incubated for 2 h at RT with human serum albumin in 2% Marvell/CPA buffer (10 mM sodium citrate+10 mM sodium phosphate+10 mM sodium acetate+115 mM NaCl) adjusted to pH 7.3. Unbound phages were removed by 10 washes with CPA/0.05% Tween20 (CPAT) pH7.3, followed by 2 washes with CPAT pH5.8. Bound phage was eluted with CPA pH5.8 for 30 min at RT and neutralized with 1M Tris-HCl pH 7.

In a third selection strategy, phage libraries were incubated for 2 h at RT with human serum albumin in 2% Marvell/CPA pH5.8. Unbound phages are removed by 10 washes with CPAT pH5.8, followed by 2 washes with CPA pH 7.3. Bound phage is eluted with 1 mg/ml trypsin/CPA pH 7.3 for 30 min at RT.

In a fourth selection strategy, phage libraries were incubated for 2 h at RT with human serum albumin in 2% Marvell/PBS pH5.8. Unbound phages are removed by 10 washes with PBST pH5.8, followed by 2 washes with PBSpH 7.3. Bound phage was eluted with 1 mg/ml trypsin/CPA pH 7.3 for 30 min at RT.

In all selections, enrichment is observed. The output from each selection was re-cloned as a pool e.g. into the expression vector pAX51. Colonies are picked and grown in 96 deep-well plates (1 ml volume) and induced by adding IPTG for Nanobody expression. Periplasmic extracts (volume: ˜80 μl) are prepared according to standard methods (see for example the prior art and applications filed by applicant cited herein).

### Example 29

### Screening of Nanobody Repertoire for the Occurrence of a pH-Sensitive Interaction via Surface Plasmon Resonance (BIAcore)

Human serum albumin was immobilized on a CM5 sensor chip surface via amine coupling using NHS/EDC for activation and ethanolamine for deactivation (Biacore amine coupling kit)

Approximately 1000RU of human serum albumin was immobilized. Experiments were performed at 25° C. The buffers used for the pH dependent binding of Nanobodies to albumin (Biacore) are as follows: 10 mM Sodium citrate (Na₃C₆H₅O₇)+10 mM Sodium phosphate (Na₂HPO₄)+10 mM Sodium Acetate (CH₃COONa)+115 mM NaCl. This mixture is brought to pH7, pH6 and pH5 by adding HCl or NaOH (dependent on the pH of the mixture measured).

Periplasmic extracts were diluted in running buffers of pH7, pH6 and pH5. The samples were injected for 1 min at a flow rate of 45 ul/min over the activated and reference surfaces. Those surfaces were regenerated with a 3s pulse of glycine-HCl pH1.5+0.1% P20. Evaluation was done using Biacore T100 evaluation software.

The off rate of different Nanobodies at pH7 and pH5 is documented in Table E-1. The majority of the Nanobodies (4A2, 4A6, 4B5, 4B6, 4B8, 4C3, 4C4, 4C5, 4C8, 4C9, 4D3, 4D4, 4D7 ad 4D10 have a faster off rate at pH 5 compared with pH 7 (2-6 fold difference in off rate). The Nanobody 4A9 has a slower off-rate at pH 5 compared to pH 7 (0.54 fold difference in off rate). For other Nanobodies including 4C12, 4B1, 4B10, IL6R202, Alb-8, and 4D5, binding to antigen does not change at different pH.

Direct screening of nanobody repertoires for conditional binding to antigen can thus be used.

TABLE E-1
Off rate (determined by Biacore) of different
Nanobodies at pH 7 and pH 5 is documented

| Nanobody | kd (1/s) at pH 7 | kd (1/s) at pH 5 | Ratio pH 7/pH 5 |
|---|---|---|---|
| 4D10 | 5.23E−04 | 3.41E−03 | 6.52 |
| 4A6 | 1.73E−03 | 9.99E−03 | 5.77 |
| 4C9 | 4.41E−04 | 1.71E−03 | 3.88 |
| 4A2 | 6.42E−03 | 2.27E−02 | 3.54 |
| 4C8 | 6.24E−04 | 2.09E−03 | 3.35 |
| 4C3 | 1.12E−03 | 3.75E−03 | 3.35 |
| 4B6 | 3.68E−04 | 1.19E−03 | 3.23 |
| 4D4 | 6.02E−03 | 1.66E−02 | 2.76 |
| 4C5 | 5.41E−04 | 1.32E−03 | 2.44 |
| 4B8 | 7.41E−04 | 1.80E−03 | 2.43 |
| 4C4 | 4.99E−04 | 1.21E−03 | 2.42 |
| 4D3 | 5.65E−03 | 1.37E−02 | 2.42 |
| 4D7 | 6.53E−04 | 1.58E−03 | 2.42 |
| 4B5 | 1.74E−03 | 4.03E−03 | 2.32 |
| 4D5 | 2.04E−02 | 2.63E−02 | 1.29 |
| 4C11 | 2.63E−02 | 3.12E−02 | 1.19 |
| 4B1 | 8.75E−03 | 7.73E−03 | 0.88 |
| 4B10 | 4.99E−02 | 4.34E−02 | 0.87 |
| 4A9 | 1.30E−02 | 7.01E−03 | 0.54 |
| Alb8 | 2.97E−03 | 2.78E−03 | 1.07 |
| IL-6R202 | 4.08E−03 | 6.19E−03 | 1.52 |

### Example 30

### Screening for Conditional Binding of Nanobodies by ELISA

To screen Nanobodies for their conditional binding to albumin, a binding ELISA can also be performed with two representative conditions, pH 5.8 and pH7.3 and the relative binding strength determined. Maxisorb micro titer plates (Nunc, Article No. 430341) were coated overnight at 4° C. with 100 μl of a 1 μg/ml solution human serum albumin in bicarbonate buffer (50 mM, pH 9.6). After coating, the plates were washed three times with PBS containing 0.05% Tween20 (PBST) and blocked for 2 hours at room temperature (RT) with PBS containing 2% Marvel (PBSM). After the blocking step, the coated plates were washed 2 times with PBST pH 5.8, and a ten-fold dilution aliquot of each periplasmic sample in PBSM pH5.8 (100 μl) is transferred to the coated plates and allowed to bind for 1 hour at RT. After sample incubation, the plates were washed five times with PBST and incubated for 1 hour at RT with 100 μl of a 1:1000 dilution of mouse anti-myc antibody in 2% PBSM. After 1 hour at RT, the plates were washed five times with PBST and incubated with 100 μl of a 1:1000 dilution of a goat anti-mouse antibody conjugated with horseradish peroxidase. After 1 hour, plates were washed five times with PBST and incubated with 100 μl of slow TMB (Pierce, Article No. 34024). After 20 minutes, the reaction was stopped with 100 μl H₂SO₄. The absorbance of each well was measured at 450 nm.

92 periplasmic extracts for each of the conditional selection strategies described herein, are analyzed in this ELISA. Table E-2 depicts the result for Nanobodies that conditionally bind to human serum albumin at neutral pH, i.e. pH 7.4, but not to acidic, i.e. pH 5.8. Table E-3 depicts the results for Nanobodies that conditionally bind to human serum albumin at acidic pH, i.e. pH 5.8, but not to neutral pH, i.e. pH 7.4.

Upon 1 round of selection on human serum albumin, followed by total elution, Nanobodies are identified that either conditionally bind to albumin at acidic pH (n=16) or at neutral pH (n=19). Driving the selection conditions towards conditional binding, results in a higher ratio of conditionally binding nanobodies (n=23).

TABLE E-2
Nanobodies (Clones) that only bind in neutral
conditions but not in acidic condition

| | | | Selection | ELISA results | |
|---|---|---|---|---|---|
| PMP | Clone ID | llama | conditions | Acidic | Neutral |
| HLEPMP14 | B5 | 117 | acidic with trypsin | 0.0180 | 0.1450 |
| | B6 | 117 | elution | 0.0130 | 0.0970 |
| | F6 | 117 | | 0.0150 | 0.1070 |
| HLEPMP16 | B1 | 117 | acidic with trypsin | 0.0250 | 0.1590 |
| | C1 | 117 | elution | 0.0300 | 0.2540 |
| | D1 | 117 | | 0.0110 | 0.7630 |
| | F1 | 117 | | 0.0230 | 0.1870 |
| | C2 | 117 | | 0.0270 | 0.4200 |
| | D2 | 117 | | 0.0570 | 0.6620 |
| | E2 | 117 | | 0.0830 | 0.6590 |
| | F2 | 117 | | 0.0270 | 0.5080 |
| | G2 | 117 | | 0.0100 | 0.1960 |
| | B3 | 117 | | 0.0110 | 0.2720 |
| | C3 | 117 | | 0.0220 | 0.3070 |
| | D3 | 117 | | 0.0480 | 0.6080 |
| | E3 | 117 | | 0.0380 | 0.4530 |
| | F3 | 117 | | 0.0460 | 0.2980 |
| | F4 | 117 | | 0.0290 | 0.2470 |
| | G4 | 117 | | 0.0540 | 0.3530 |

TABLE E-3
Clones that only bind in acidic conditions
but not in neutral condition

| | | | Selection | ELISA results | |
|---|---|---|---|---|---|
| PMP | Clone ID | llama | conditions | Acidic | Neutral |
| HLEPMP14 | E1 | 117 | acidic with | 0.1180 | 0.0250 |
| | B2 | 117 | trypsin elution | 0.1130 | 0.0260 |
| | H4 | 117 | | 0.0880 | 0.0260 |
| | B8 | 117 | neutral with | 0.0970 | 0.0260 |
| | B9 | 117 | acidic buffer | 0.1160 | 0.0210 |
| | F9 | 117 | elution | 0.1140 | 0.0230 |
| | G9 | 117 | *PBS buffer @ | 0.1660 | 0.0160 |
| | B10 | 117 | pH 5.8 | 0.1210 | 0.0110 |
| | D10 | 117 | | 0.1100 | −0.0110 |
| HLEPMP15 | D1 | 118 | acidic with | 0.1120 | 0.0180 |
| | E1 | 118 | trypsin elution | 0.1290 | 0.0250 |
| | D2 | 118 | | 0.0780 | 0.0160 |
| | E2 | 118 | | 0.1890 | 0.0290 |
| | F2 | 118 | | 0.1040 | 0.0180 |
| | G2 | 118 | | 0.0820 | 0.0180 |
| | E3 | 118 | | 0.1840 | 0.0220 |
| | F3 | 118 | | 0.0970 | 0.0170 |
| | G3 | 118 | | 0.0920 | 0.0160 |
| | B4 | 118 | | 0.1050 | 0.0220 |
| | G4 | 118 | | 0.1510 | 0.0250 |
| | E6 | 118 | | 0.1080 | 0.0170 |
| | F6 | 118 | | 0.1080 | 0.0310 |
| | G7 | 118 | neutral with | 0.1740 | 0.0280 |
| | G8 | 118 | acidic buffer | 0.0700 | 0.0170 |
| | A9 | 118 | elution | 0.1460 | 0.0330 |
| | F10 | 118 | *PBS buffer @ | 0.1090 | 0.0340 |
| | | | pH 5.8 | | |
| HLEPMP16 | D7 | 117 | neutral with | 0.1550 | 0.0550 |
| | F7 | 117 | acidic buffer | 0.1070 | 0.0120 |
| | G7 | 117 | elution | 0.1880 | 0.0220 |
| | E8 | 117 | *CPA buffer @ | 0.1120 | 0.0270 |
| | F8 | 117 | pH 5.8 | 0.1270 | 0.0210 |
| | G8 | 117 | | 0.2060 | 0.0250 |
| | E9 | 117 | | 0.1190 | 0.0100 |
| | F9 | 117 | | 0.2620 | 0.0410 |
| | G9 | 117 | | 0.2660 | 0.0150 |
| | E10 | 117 | | 0.1410 | 0.0110 |
| | F10 | 117 | | 0.3590 | 0.0100 |
| | G10 | 117 | | 0.4450 | 0.0600 |
| | H10 | 117 | | 0.0840 | 0.0140 |
| | E11 | 117 | | 0.3050 | 0.0130 |
| | F11 | 117 | | 0.4250 | 0.0170 |
| | G11 | 117 | | 0.1680 | 0.0170 |

TABLE E-4
Sequences of albumin binder

4A1- SEQ ID NO: 28
EVQLVESGGGLVQPEGSLRLSCRASGSIFSINTMGWYRQPPGKEREF
VARIYPGITHYADSVKGRFTISRDNAKNTVYLQMNSLKPEDTAVYYC
FYYYDDRNYWGEGTLVTVSS
4A2- SEQ ID NO: 29
EVQLVESGGGLVQAGGSLRLSCAASGLSFSSYAMGWFRQAPGKEREF
VAAIRRREGNSYYADSVKGRFTISRDSAKNTVYLQMNSLKPEDTALY
SCAATAPHYSGSFAYAGGYDYWGQGTQVTVSS
4A6- SEQ ID NO: 30
EVQLVESGGGLVQPGGSLRLSCAASPFTLDYYAIGWFRQAPGKEREG
VSCSTSHGKTYHADSVKGRFTISRDNAKINTVYLQMNSLKPEDTAVY
YCAAGACMGGSGYEADFGSWGQGTQVTVSS
4A9- SEQ ID NO: 31
EVQLVESGGGLVQAGGSLRLSCEASGFTLDYSGVGWFRQAPGKEREL
VSCISRGGDRAGYANSVKGRFTMSRDNAKNILYLQMNSLKPEDTAVY
YCAATHSGSGCYDGAIDYWGKGTLVTVSS
4B1- SEQ ID NO: 32
EVQLVESGGGLVQPGGSLRLSCVAAGFTLDYYAIGWFRQAPGKEREG
VSCITSDGRTYYADSVKGRFTISRDMAKKMVYLQMNSLKPEDTAVYY
CAAGACMGGSGYEADFGSWGQGTQVTVSS
4B5- SEQ ID NO: 33
EVQLVESGGGLVQAGDSLRLSCAASGRTYSRNAMAWFRQAPGKEREF
VAGIDWSSENTRYIDSVKGRFTISRDNAKSTMYLQMNSLKPEDTAVY
YCAAGTSWGALASRLEAAYSSWGQGTQVTVSS
4B6- SEQ ID NO: 34
KVQLVESGGGLVQVGGSLRLSCVASGRTYGGNAMAWFRQAPGKEREF
VAGIDWSSENTRYTDSVKGRFTISRDNAKNTMYLQMNSLKPEDTAVY
YCAAGTSWGALASRLENAYSAWGQGTQVTVSS
4B8- SEQ ID NO: 35
EVQLVESGGGLVQAGGSLRLSCAASGGTYSGNAMAWFRQAPGKEREF
VAGIDWSSENTRYIDSVKGRFTISRDNAKNTMYLQMNSLKPEDTAVY
YCAAGTSWGALASRLEAAYSSWGQGTQVTVSS
4B10- SEQ ID NO: 36
EVQLVESGGGLVQAGDSLRLSCAASGRTFSSYAMGWFRQAPGTERQF
VARITGKGDSTDYADSVRGRFTISRDNAKNTVYLQMNSLKPEDTAVY
YCAADAFNSLLQAGRAEYWGQGTQVTVSS
4C3- SEQ ID NO: 37
EVQLVESGGGLVQAGGSLRLSCAASGRTFSSYAMAWFRQAPGEEREF
VATISVSGGYTYYADSVKGRFTISRDNAKNTVYLQMNTLKPEDTAVY
YCAAGDSSSWLEHMYDYWGQGTQVTVSS
4C4- SEQ ID NO: 38
EVQLVESGGGLVQAGDSLRLSCAASGRPFMSYVMGWFRRAPGKEREF
VGGINWGSGNTWYTDSVLGRFTISRDNAKNTVYLQMNSLKPEDTAVY
YCATAAGVGYYRYERQYDYWGQGTQVTVSS
4C5- SEQ ID NO: 39
EVQLVESGGGLVQAGDSLRLSCAASGRPFSAYVMGWFRRAPGKEREF
VGGINWNSANTWYTDSVLGRFTISKDNAKNTVYLQMNSLKPEDTAVY
YCAAAGGVGYYRYERQYDYWGQGTQVTVSS
4C8- SEQ ID NO: 40
EVQLVESGGGLVQAGDSLRLSCAASGRTYTPYVMGWFRRAPGKEREF
VGAVSWSGTNTWYTDSVKGRFTISRDNAKNTVYLQMNSLKPEDTAVY
YCAAGDGVGIYRYEHQYDYWGQGTQVTVSS
4C9- SEQ ID NO: 41
EVQLVESGGGLVQAGDSLRLSCTASERPFSTYVMGWFRPAPGKEREF
VGGITWSGINAWYTDSVLGRFTISSDNAKNTVYLQMNSLKPEDTAVY
YCAAASGVGRYRYELQYDYWGQGTQVTVSS
4C11- SEQ ID NO: 42
EVQLVESGGGLVQAGGSLRLSCAASGLSFSRYAMGWFRQAPGKQREY
VAVISSSDTTYYTNSAKGRFTISRDNALNTVYLQMNSLKPEDTAVYF
CAADSFVTALQTLTQINYWGQGTQVTVSS
4D3- SEQ ID NO: 43
EVQLVKSGGGLVQPGGSLRLSCAASGFTFSHYQMSWVRQAPGKDVEW
VSSISMLGGGTTYADSVKGRFTISRDNAKSTLVLQMNNLKVEDTAVY
YCARGFSGNYYRADLGQGTQVTVSS
4D4- SEQ ID NO: 44
EVQLVESGGGLVQAGDSLRLSCAASGRTFSPYVMGWFRRAPGKEREF
VGGINWSGSNTWYTDSVKGRFTISRDNVKNMVYLQMNSLKPEDTAVY
YCAAGSGVGMYRYERQYDYWGQGTQVTVSS
4D5- SEQ ID NO: 45
EVQLVESGGGLVQAGGSLRLSCAASGLSFSKYAMGWFRQAPGKQREY
VAVISSSDTTYYTNSAKGRFTISRDNAENTVYLQMNSLKPEDTAVYF
CAADSYVTALQTLTQISYWGQGTQVTVSS
4D7- SEQ ID NO: 46
EVQLVESGGGLVQAGDSLRLSCAASGRPFSSYVMGWFRRAPGKEREF
VGGINWNSGNTWYSDSVLGRFTISRDNAKNTVDLQMNSLKPEDTAVY
YCATASGVGYYRYERQYDYWGQGTQVTVSS
4D10- SEQ ID NO: 47
EVQLVESGGGLVQAGDSLRLSCTASERPFMSYVMGWFRRAPGKDREF
VGAITWSGINTWYSDSVLGRFTISRDNAKNTVYLQMNSLKPEDTAVY
YCAVADGVGLYRYERQYDYWGQGTQVTVSS
IL6R202- SEQ ID NO: 48
EVQLVESGGGLVQPGGSLRLSCAASGFTFSDYDIGWIFRQAPGKGRE
GVSGISSSDGNTYYADSVKGRFTISRDNAKNTLYLQMNSLRPEDTAV
YYCAAEPPDSSWYLDGSPEFFKYWGQGTLVTVSSGGGGSGGGSEVQL
VESGGGLVQPGNSLRLSCAASGFTFSSFGMSWVRQAPGKGLEWVSSI
SGSGSDTLYADSVKGRFTISRDNAKTTLYLQMNSLRPEDTAVYYCTI
GGSLSRSSQGTLVTVSS
ALB-8- SEQ ID NO: 49
EVQLVESGGGLVQPGNSLRLSCAASGFTFSSFGMSWVRQAPGKGLEW
VSSISGSGSDTLYADSVKGRETISRDNAKTTLYLQMNSLRPEDTAVY
YCTIGGSLSRSSQGTLVTVSS
IL6R-4D10- SEQ ID NO: 50
EVQLVESGGGLVQPGGSLRLSCAASGFTFSDYDIGWFRQAPGKGREG
VSGISSSDGNTYYADSVKGRFTISRDNAKNTLYLQMNSLRPEDTAVY
YCAAEPPDSSWYLDGSPEFFKYWGQGTLVTVSSGGGGSGGGSEVQLV
ESGGGLVQAGDSLRLSCTASERPFMSYVMGWFRRAPGKDREFVGAIT
WSGINTWYSDSVLGRFTISRDNAKNTVYLQMNSLKPEDTAVYYCAVA
DGVGLYRYERQYDYWGQGTQVTVSS

### Example 31

### Analysis of Effect of Conditional Binding on Pharmacokinetic Behaviour of the Polypeptide of the Invention

### a) Construction of Bi- or Multispecific Nanobody Format

Bispecific nanobodies are e.g. generated by construction of a C-terminal pH dependent FcRn binding Nanobody, a 9 amino acid Gly/Ser linker (e.g. GGGGSGGGS) and an N-terminal anti-target Nanobody, e.g. an N-terminal Polypeptide with 2 Nanobodies against EPO-R functioning as agonist on human or murine EPO-R or an N-terminal Polypeptide with 2 Nanobodies against EPO-R functioning as agonist on human or murine GHR. These constructs may be expressed in E. coli as c-myc, His6-tagged proteins and subsequently purified from the culture medium by immobilized metal affinity chromatography (IMAC) and size exclusion chromotagraphy (SEC).

### b) Retention of Conditional Binding Upon Formatting into Multispecific Format

The conditional pH-binding properties of the anti-FcRn or pIgR Nanobody or dAbs within the multispecific nanobody formats (e.g. are evaluated via surface plasmon resonance (BIAcore), e.g. a conditional binder as disclosed in this application is linked to one or more nanobody or dAbs binding to one or more protein target(s), e.g. is linked to 2 Nanobodies directed against Epo-R or HGR. Cross-reactivity to cynomolgus serum albumin is also assessed. Human and cynomolgus FcRn or pIgR are immobilized on a CM5 sensor chip surface via amine coupling using NHS/EDC for activation and ethanolamine for deactivation (Biacore Amine Coupling Kit)

Experiments are performed at 25° C. The buffers used for the pH dependent binding of Nanobodies to FcRn or pIgR (Biacore) are as follows: 10 mM Sodium citrate (Na₃C₆H₅O₇)+10 mM Sodium phosphate (Na₂HPO₄)+10 mM Sodium Acetate (CH₃COONa)+115 mM NaCl. This mixture is brought to pH7, pH6 and pH5 by adding HCl or NaOH (dependent on the pH of the mixture measured).

Purified Polypeptides are diluted in running buffers of pH7, pH6 and pH5. The samples are injected for 1 min at a flow rate of 45 ul/min over the activated and reference surfaces. Those surfaces are regenerated with a 3s pulse of glycine-HCl pH1.5+0.1% P20. Evaluation is done using Biacore T100 evaluation software.

### Example 32

### Pharmacokinetic Profile of Multispecific Nanobody Formats in Cynomolgus Monkey Delivered by I.V. Injection

A pharmacokinetic study is conducted in cynomolgus monkeys. A Polypeptide of the Invention (e.g. Epo-R or HGR agonstic bivalent polypeptide with FcRn or pIgR pH dependent binding block, i.e. 2 Epo-R or 2 HGR binding blocks linked via a 9 amino acid Gly/Ser linker to each other and a conditional FcRn or pIgR binding block, again linked e.g. via a 9 amino acid Gly/Ser linker) is administered intravenously by bolus injection (1.0 ml/kg, approximately 30 sec) in the vena cephalica of the left or right arm to obtain a dose of 2.0 mg/kg. The Nanobody concentration in the plasma samples is determined via ELISA.

The concentration in the plasma samples is determined as follows:

Maxisorb micro titer plates (Nunc, Article No. 430341) are coated overnight at 4° C. with 100 μl of a 5 μg/ml solution of the Polypeptide of the Invention in bicarbonate buffer (50 mM, pH 9.6). After coating, the plates are washed three times with PBS containing 0.1% Tween20 and blocked for 2 hours at room temperature (RT) with PBS containing 1% casein (250 μl/well). Plasma samples and serial dilutions of polypeptide-standards (spiked in 100% pooled blank cynomolgus plasma) are diluted in PBS in a separate non-coated plate (Nunc, Article No. 249944) to obtain the desired concentration/dilution in a final sample matrix consisting of 10% pooled cynomolgus plasma in PBS. All pre-dilutions are incubated for 30 minutes at RT in the non-coated plate. After the blocking step, the coated plates are washed three times (PBS containing 0.1% Tween20), and an aliquot of each sample dilution (100 μl) is transferred to the coated plates and allowed to bind for 1 hour at RT. After sample incubation, the plates are washed three times (PBS containing 0.1% Tween20) and incubated for 1 hour at RT with 100 μl of a 100 ng/ml solution of sIL6R in PBS (Peprotech, Article No. 20006R). After 1 hour at RT, the plates are washed three times (PBS containing 0.1% Tween20) and incubated with 100 μl of a 250 ng/ml solution of a biotinylated polyclonal anti-Polypeptide of the Invention in PBS containing 1% casein (R&D systems, Article No. BAF227). After incubation for 30 minutes (RT), plates are washed three times (PBS containing 0.1% Tween20) and incubated for 30 minutes (RT) with 100 μl of a 1/5000 dilution (in PBS containing 1% casein) of streptavidine conjugated with horseradish peroxidase (DaktoCytomation, Article No. P0397). After 30 minutes, plates are washed three times (PBS containing 0.1% Tween20) and incubated with 100 μl of slow TMB (Pierce, Article No. 34024). After 20 minutes, the reaction is stopped with 100 μl HCl (1N). The absorbance of each well is measured at 450 nm (Tecan Sunrise spectrophotometer), and corrected for absorbance at 620 nm. This assay measures free Polypeptide of the Invention as well as Polypeptide of the Invention bound Polypeptide of the Invention. Concentration in each plasma sample is determined based on a sigmoid standard curve with variable slope of the respective Polypeptide of the Invention.

Each individual plasma sample is analyzed in two independent assays and an average plasma concentration is calculated for pharmacokinetic data analysis.

All parameters are calculated with two-compartmental modeling, with elimination from the central compartment.

### Example 33

### Preparation of Various Pharmaceutical Orally Deliverable Compositions

### a) Capsules Comprising the Polypeptides of the Invention—Preferably Enteric Coated Capsules

For the purposes of illustrating this invention in e.g. a monkey or mouse, the enteric coating material is selected from HPMC-AS (pH 5.5), CAT (pH 5.5) and Eudragit L (pH 5.5), most preferably Eudragit L (pH 5.5).

For use in the human, the enteric coating material preferably may be one which will provide for release of polypeptide at about pH 6.0-6.5 such as, for example, CAP and HPMC-AS.

The enterocoating is carried out by methods known per se in the art, e.g., according to Remington Pharmaceutical Sciences, p. 1614-1615 (1975, 15th Ed., Mack Pub. Co.) and Theory and Practice of Industrial Pharmacy, Lackman, Liberman & Caning, p. 116-117, 371-374 (1976, 2nd Ed.). The enteric micro-encapsulation process is also known (Theory and Practice of Industrial Pharmacy ibid, pp. 420-438). See also Remington Pharmaceutical Sciences, p. 1637 (1985, 17th Ed., Mack Pub. Co.). Typically, the amount of enteric coating material used preferably is in the range about 10-20 mg per 500 cm.sup.2 of surface area of capsule or tablet, especially of capsule as produced in the actual examples described herein below. The amount of enteric coating material broadly is in the range of about 1-5000 mg/capsule, more preferably about 10-1000 mg/capsule, most preferably about 50-100 mg/capsule.

A solution comprising the Polypeptides of the Invention (e.g. the herein described examples, e.g. agonistic HGR or EpoR polypeptides (i.e. bispecific construct comprising 2 Nanobodies against HGR or EpoR including construct additionally comprising a FcRn or pIgR binding Nanobodies (preferably pH dependent binding, e.g. binding at pH 6 or less but not or to a much lower extend at pH 7 and more)) is filled up into enteric coated capsules and used within a short time, e.g. within a week or day for the in vivo experiment as e.g. presented in the below examples.

A liquid formulation will generally consist of a solution or suspension containing the biologically active polypeptide, e.g. the Polypeptide of the Invention and optionally protease inhibitor(s) filled into a pharmaceutically acceptable capsule for example, a standard or conventional hard gelatin capsule and the filled capsule will be coated, e.g. as described above. The solution or suspension which is filled into such capsule will generally consist of the biologically active Polypeptide of the Invention and protease inhibitor(s) dissolved or suspended in any pharmaceutically acceptable liquid carrier such as, for example, a sterile aqueous carrier or water-miscible solvents such as, for example, ethanol, glycerin, propylene gylcol and sorbitol, or mixtures of any of the foregoing.

### Example 34

### In Vivo Model to Test Systemic Delivery

### In Vivo Model for Epo-R Agonist Read Out (Spiekermann et al, 2002, Supra)

Female BALB/c mice 4-6 wk of age and control C57BL/6 mice from e.g. The Jackson Laboratory are maintained under pathogen-free conditions. Mice are anaesthetized with e.g. Isoflurane by inhalation and the different Polypeptides of the Invention (e.g. as disclosed above, e.g. construct comprising 2 anti-mouse non-neutralizing Epo-R Nanobodies (e.g. with 9 Gly linker), optionally comprising a pH independent or pH dependent anti-mouse FcRn or pIgR Nanobody (i.e. binding at gut pH, about pH 6, but released at blood pH7 or more) are injected intraperitoneally, gauged into small intestine, fed intragastrically using a ball-point needle (once, twice, or four times 12 h apart), or administered orally by a enterically coated capsule for mouse consumption, e.g. capsule from example 32. Mice are killed by CO₂ inhalation 8 h or 4 d later and whole blood is obtained by cardiac puncture.

### Flow Cytometric Analysis

Whole blood samples from above are added to e.g. ReticOne Reagent according to the manufacturer's instructions. Flow cytometry is performed with e.g. a Coulter Epics XL machine. Acquisition parameters are calibrated each time by e.g. Retic-Cal Biological Calibration and Retic-C Cell Control. 40,000 total events in the red blood cell gate are acquired and analyzed with ReticOne automated software for percentage of reticulocytes (all materials e.g. from Beckman Coulter). Increase in number of reticulocytes in blood is indicative of functional delivery of Epo-R agonists into body, i.e. systemic delivery.

Note: For Epo-R dimerization, the agonistically acting Nanobody construct may have a Koff equal or lower than 1 nM since interaction of the first site of Epo for EpoR is of high affinity (dissociation constant 1 nM) while the second binding interaction is much weaker (1 uM). To be determined e.g. in BioCore experiments.

### In Vivo Model for HGR Agonist Read Out

In vivo assay to test GHR agonists (Wang et al., 1996 Molecular and Cellular Endocrinology, Volume 116, Issue 2, 5 Feb. 1996, Pages 223-226)
Studies on promotion of animal growth using GH deficient hypophysectomized rats.

Similarly as above, mice are anaesthetized with e.g. Isoflurane by inhalation and the different Polypeptides of the Invention (e.g. as disclosed above, e.g. construct comprising 2 anti-mouse non-neutralizing GHR Nanobodies (e.g. with 9 Gly linker), optionally comprising a pH independent or pH dependent anti-mouse FcRn or pIgR Nanobody (i.e. binding at gut pH, about pH 6, but released at blood pH7 or more) are a) injected intraperitoneally, b) gauged into small intestine, c) fed intragastrically using a ball-point needle (once, twice, or four times 12 h apart), or d) administered orally by e.g. a enterically coated capsule acceptable for mouse consumption, e.g. capsule from example 32. Mice growth is monitored.

Increase in growth is indicative of a systemically delivered GHR agonist.

Note: For GHR, the agonistically acting Nanobody construct against GHR from above may have a Koff equal or lower than 0.3 nM since interaction of GH to GHR-dimer was reported to be in the range of 0.3 nM (Cunningham et al, 1989, Science 244:1081-1085.). To be determined e.g. in BioCore experiments.

### Preferred Embodiments

1. A pharmaceutical composition for oral administration comprising a therapeutically effective amount of a polypeptide comprising one or more single variable domain(s) and a pharmaceutically acceptable enteric coating.
2. The composition according to embodiment 1, wherein said polypeptide comprises or essentially consists of a single Nanobody, domain antibody, single domain antibody or “dAb”, preferably a Nanobody.
3. The composition according to embodiment 1, wherein said polypeptide comprises or essentially consists of at least two Nanobodies, domain antibodies, single domain antibodies or “dAbs”, preferably a Nanobody.
4. The composition according to embodiment 3, wherein said polypeptide comprises or essentially consists of at least one Nanobody, domain antibody, single domain antibody or “dAb” against one epitope, antigen, target, protein or polypeptide and at least one other Nanobody, domain antibody, single domain antibody or “dAb” directed against another epitope, antigen, target, protein or polypeptide.
5. The composition according to embodiment 1, wherein said polypeptide comprises one or more Nanobodies, domain antibodies, single domain antibodies or “dAbs” linked to one or more amino acid sequence that provides an increased half-life following delivery to the subject.
6. The composition according to embodiment 5, wherein said one or more amino acid sequence is a Nanobody, a domain antibody, a single domain antibody or a “dAb”, preferably a Nanobody.
7. The composition according to embodiment 6, wherein said one or more amino acid sequence is directed against a serum protein.
8. The composition according to embodiment 1, wherein said polypeptide comprises one or more Nanobodies, domain antibodies, single domain antibodies or “dAbs” linked to one or more amino acid sequences that allow the resulting polypeptide to cross the epithelial membrane of the gut.
9. The composition according to embodiment 8, wherein said one or more amino acid sequences is a Nanobody, a domain antibody, a single domain antibody or a “dAb” preferably a Nanobody.
10. The composition according to embodiment 1, wherein said polypeptide comprises a therapeutic polypeptide or agent linked to a Nanobody, a domain antibody, a single domain antibody or a “dAb” directed against an epithelial trans-membrane protein on the intestinal membrane.
11. The composition according to any of embodiments 1 to 10, wherein said Nanobody, domain antibody, single domain antibody or “dAb” is derived from a V_{H} or V_{HH}.
12. The composition according to embodiment 11, wherein said Nanobody, domain antibody, single domain antibody or “dAb” is a humanized V_{HH} or a camelized V_{H}.
13. The composition according to any of embodiment 1 to 12, which, upon oral administration to a subject, induces a systemic therapeutic or biological response in said subject.
14. The composition according to any of embodiments 1 to 13, wherein, upon oral administration of said composition to a subject, the polypeptide reaches the bloodstream with a Cmax of at least 1 ng polypeptide or protein per ml of blood following oral administration of a dose of 5 mg/kg body weight of said polypeptide.
15. The composition according to any of embodiments 1 to 14, wherein, upon oral administration of said composition to a subject, the polypeptide reaches a Cmax in blood of at least 1% of the Cmax that is reached following parenteral administration of the same amount of polypeptide.
16. The composition according to any of embodiments 1 to 15, wherein, upon oral administration of said composition to a subject, the polypeptide reaches the bloodstream with a Tmax of less than 120 minutes following oral administration of said composition to said subject.
17. The composition according to any of embodiments 1 to 16, wherein, upon oral administration of said composition to a subject, the polypeptide reaches the bloodstream with a Cmax of at least 1 ng polypeptide per ml of blood within less than 120 minutes following oral administration of a dose of 5 mg/kg body weight of said polypeptide to said subject.
18. The composition according to any of embodiments 1 to 17, wherein, upon oral administration of said composition to a subject, the AUC for the polypeptide in blood is at least 500 ng/ml/minute polypeptide following oral administration of a dose of 5 mg/kg body weight of said polypeptide to said subject.
19. The composition of any of embodiments 1 to 18, wherein, upon oral administration of said composition to a subject, the polypeptide has an absolute and/or relative bioavailability in blood of at least 1%.
20. The composition according to any of embodiment 1 to 12, which, upon oral administration to a subject, induces a therapeutic or biological response in the gut of said subject.
21. The composition according to embodiment 20, wherein, upon oral administration of said composition to a subject, the polypeptide reaches a Cmax in the gut of at least 1% of the Cmax that is reached following parenteral administration of the same amount of polypeptide.
22. The composition according to any of embodiments 20 or 21, wherein, upon oral administration of said composition to a subject, the polypeptide reaches the gut with a Tmax of less than 120 minutes following oral administration of said composition to said subject.
23. The composition of any of embodiments 20 to 22, wherein, upon oral administration of said composition to a subject, the polypeptide or protein has an absolute and/or relative bioavailability in the gut of at least 1%, 2%, 3%, 4%, 5%, 6%, 7%, 8%, 9%, or 10%, preferably 10%.
24. The composition of any of embodiments 1 to 23, wherein the enteric coating is an anionic polymer and dissolve at ranges from pH 5.5. to pH 7.
25. The composition of any of embodiments 1 to 24, wherein the enteric coating is an anionic polymer comprising methacrylic acid and methacrylates and dissolve at ranges from pH 5.5. to pH 7.
26. The composition of any of embodiments 1 to 25, additionally comprising a permeability enhancer such as e.g. acylcarnitine or N-(5-chlorosalicyloyl)-8-aminocaprylic acid.
27. The composition of any of embodiments 1 to 26, additionally comprising protease inhibitor such as e.g. organic acids.
28. The composition of any of embodiments 1 to 27, wherein the polypeptide comprises at least one Nanobody.
29. The composition of any of embodiments 1 to 28, wherein the polypeptide comprises at least one Llama-derived Nanobody.
30. The composition of any of embodiments 1 to 29, wherein the polypeptide comprises at least one Llama-derived Nanobody which is humanized.
31. The composition of any of embodiments 1 to 30, wherein the polypeptide comprises a) at least one Nanobody directed against a target molecule; and b) a Nanobody directed against the extracellular part of FcRn.
32. The composition of any of embodiments 1 to 29, wherein the polypeptide comprises a) at least one Nanobody directed against a target molecule; and b) a Nanobody directed against the extracellular part of pIgR.
33. The composition of any of embodiments 1 to 29, wherein the polypeptide comprises a) at least one Nanobody directed against a target molecule; and b) a Nanobody directed against the extracellular part of Vit B12 receptor.
34. The composition of embodiment 31, wherein the polypeptide comprises a) at least one Nanobody directed against a target molecule; and b) a Nanobody directed against the extracellular part of FcRn, preferably with a Kd of 100 nM, 10 nM, 1 nM, 100 pM or 10 pM, more preferably a Kd of 10 nM or 1 nM, e.g. a Kd of 10 nM.
35. The composition of embodiment 32, wherein the polypeptide comprises a) at least one Nanobody directed against a target molecule; and b) a Nanobody directed against the extracellular part of pIgR, preferably with a Kd of 100 nM, 10 nM, 1 nM, 100 pM or 10 pM, more preferably a Kd of 10 nM or 1 nM, e.g. a Kd of 10 nM.
36. The composition of embodiment 33, wherein the polypeptide comprises a) at least one Nanobody directed against a target molecule; and b) a Nanobody directed against the extracellular part of Vit B12 receptor, preferably with a Kd of 100 nM, 10 nM, 1 nM, 100 pM or 10 pM, more preferably a Kd of 10 nM or 1 nM, e.g. a Kd of 10 nM.
37. The composition of any of embodiment 31, wherein the polypeptide comprises a) at least one Nanobody directed against a target molecule; and b) a Nanobody directed against the extracellular part of FcRn and wherein said Nanobody directed against the extracellular part of FcRn is cross-blocked by any FcRn Nanobody of SEQ ID NOs: 1 to 6.
38. The composition of embodiment 31, wherein the polypeptide comprises a) at least one Nanobody directed against a target molecule; and b) a Nanobody directed against the extracellular part of FcRn and wherein said Nanobody directed against the extracellular part of FcRn cross-blocks FcRn Nanobody of SEQ ID NOs: 1 to 6.
39. The composition of embodiment 31, wherein the polypeptide comprises a) at least one Nanobody directed against a target molecule; and b) a Nanobody directed against the extracellular part of FcRn and wherein said Nanobody directed against the extracellular part of FcRn has 70%, 75%, 80%, 85%, 90% sequence identity (measured e.g. with blast 2 sequences with blastp and scoring matrix BLOSUM62 (Henikoff & Henikoff, 1992)) to FcRn Nanobody of SEQ ID NOs: 1 to 6.
40. The composition of embodiment 32, wherein the polypeptide comprises a) at least one Nanobody directed against a target molecule; and b) a Nanobody directed against the extracellular part of pIgR and wherein said Nanobody directed against the extracellular part of pIgR is cross-blocked by pIgR Nanobody of SEQ ID NOs: 7 to 27.
41. The composition of embodiment 30, wherein the polypeptide comprises a) at least one Nanobody directed against a target molecule; and b) a Nanobody directed against the extracellular part of pIgR and wherein said Nanobody directed against the extracellular part of pIgR cross-blocks pIgR Nanobody of SEQ ID NOs: 7 to 27.
42. The composition of embodiment 30, wherein the polypeptide comprises a) at least one Nanobody directed against a target molecule; and b) a Nanobody directed against the extracellular part of pIgR and wherein said Nanobody directed against the extracellular part of pIgR has 70%, 75%, 80%, 85%, 90% sequence identity (measured e.g. with, blast 2 sequences with blastp and scoring matrix BLOSUM62 (Henikoff & Henikoff, 1992)) to pIgR Nanobody of SEQ ID NOs: 7 to 27.
43. The composition of any of embodiments 31 to 42, wherein the binding of the Nanobody directed against the extracellular part of pIgR, FcRn or Vit B12 receptor is pH dependent.
44. The composition of embodiment 43, wherein the Nanobody directed against the extracellular part of pIgR, FcRn or Vit B12 receptor binds at pH6 or lower, e.g. pH5, to its receptor and does significantly less (e.g. 2, 3, 4, 5, or 10 times or not at all) bind to its receptor at pH7 and higher.
45. The composition of any of embodiments 31 to 44, wherein the polypeptide has agonistic properties to the target molecule.
46. The composition of any of embodiments 31 to 44, wherein the polypeptide has antagonistic properties to the target molecule.
47. The composition of embodiment 45 wherein the polypeptide is an agonist to Epo-R.
48. The composition of embodiment 45 wherein the polypeptide an agonist to GHR.
49. The composition of any of embodiments 1 to 48 wherein the polypeptides are proteolytically stabilized, e.g. are selected for proteolytic stability.
50. The composition of embodiment 49 wherein the proteolytically stabilized properties are resulting from polypeptides consisting essentially of proteolytically stabilized (i.e. screened for proteolytically stabilized) Nanobodies, e.g. if consisting of more than one Nanobodies the Nanobodies are e.g. linked with Gly/Ser linkers.
51. A method for delivering a polypeptide comprising or essentially consisting of a Nanobody, a domain antibody, a single domain antibody or “dAb” to the bloodstream of a subject, said method comprising the step of orally administering a composition according to any of embodiments 1 to 50 to said subject.
52. A method for delivering a polypeptide comprising or essentially consisting of a Nanobody, a domain antibody, a single domain antibody or “dAb” to the gut of a subject, said method comprising the step of orally administering a composition according to any of embodiments 1 to 50 to said subject.
53. A method for the preparation of a composition according to any of embodiments 1 to 50.
54. A method for the prevention and/or treatment of a subject in need of polypeptide comprising or essentially consisting of a Nanobody, a domain antibody, a single domain antibody or a “dAb”, said method comprising the step of orally administering to said subject a Nanobody, a domain antibody, a single domain antibody or a “dAb” and/or a composition comprising the same.
55. A method for the prevention and/or treatment of at least one disease or disorder that can be prevented and/or treated by administering a Nanobody, a domain antibody, a single domain antibody or a “dAb” to a subject suffering from said disease or disorder, said method comprising the step of orally administering to said subject a therapeutically effective amount of said Nanobody, domain antibody, single domain antibody or “dAb”, and/or of a composition comprising the same.
56. A method for immunotherapy comprising oral administering to a subject suffering from or at risk of a diseases and/or disorders that can be cured or alleviated by immunotherapy with a Nanobody, a domain antibody, a single domain antibody or a “dAb”, a therapeutically effective amount of said Nanobody, domain antibody, single domain antibody or “dAb” and/or of a composition comprising the same.
57. Use of a Nanobody, a domain antibody, a single domain antibody or a “dAb” for the preparation of a composition according to any of embodiments 1 to 50 for the prevention and/or treatment of at least one disease or disorder that can be prevented and/or treated by orally administering to a subject a Nanobody, a domain antibody, a single domain antibody or a “dAb”.
58. The composition according to any of embodiments 1 to 50, for the prevention and/or treatment of at least one disease or disorder that can be prevented and/or treated by orally administering to a subject a Nanobody, a domain antibody, a single domain antibody or a “dAb”.
59. A method for the prevention and/or treatment of a subject in need of a Nanobody, a domain antibody, a single domain antibody or a “dAb” that is directed against a target in the kidney or bladder, said method comprising orally administering, to said subject a therapeutically effective amount of said Nanobody, domain antibody, single domain antibody or “dAb” and/or of a composition comprising the same.
60. A method for the prevention and/or treatment of at least one disease or disorder that can be prevented and/or treated by administering to a subject suffering from said disease or disorder a Nanobody, a domain antibody, a single domain antibody or a “dAb” that is directed against a target in the kidney or the bladder, said method comprising orally administering to said subject a therapeutically effective amount of said Nanobody, domain antibody, single domain antibody or “dAb” and/or of a composition comprising the same.
61. A method for the prevention and/or treatment of a disease or disorder of the kidney or bladder, said method comprising orally administering to said subject a therapeutically effective amount of a Nanobody, a domain antibody, a single domain antibody or a “dAb” that is directed against a target in the kidney or the bladder and/or of a composition comprising the same.
62. Use of a Nanobody, a domain antibody, a single domain antibody or a “dAb” directed against a target in the kidney or the bladder for the preparation of a composition according to any of embodiments 1 to 27 for the prevention and/or treatment of at least one a disease or disorder of the kidney or bladder.
63. The composition according to any of embodiments 1 to 50, wherein said Nanobody, domain antibody, single domain antibody or “dAb” is directed against a target in the kidney or the bladder for the prevention and/or treatment of a disease or disorder of the kidney or bladder.
64. A method for the prevention and/or treatment of a subject in need of a Nanobody, a domain antibody, a single domain antibody or a “dAb” that is directed against a target in the lung, said method comprising orally administering, to said subject a therapeutically effective amount of said Nanobody, domain antibody, single domain antibody or “dAb”, and/or of a composition comprising the same.
65. A method for the prevention and/or treatment of at least one disease or disorder that can be prevented and/or treated by administering to a subject suffering from said disease or disorder a Nanobody, a domain antibody, a single domain antibody or a “dAb” that is directed against a target in the lung, said method comprising orally administering to said subject a therapeutically effective amount of said Nanobody, domain antibody, single domain antibody or “dAb” and/or of a composition comprising the same.
66. A method for the prevention and/or treatment of a disease or disorder of the lung, said method comprising orally administering to said subject a therapeutically effective amount of a Nanobody, a domain antibody, a single domain antibody or a “dAb” that is directed against a target in the lung and/or of a composition comprising the same.
67. Use of a Nanobody, a domain antibody, a single domain antibody or a “dAb” directed against a target in the lung for the preparation of a composition according to any of embodiments 1 to 50 for the prevention and/or treatment of at least one disease or disorder of the lung.
68. The composition according to any of embodiments 1 to 50, wherein said Nanobody, domain antibody, single domain antibody or “dAb” is directed against a target in the lung for the prevention and/or treatment of at least one disease or disorder of the lung.
69. A method for the prevention and/or treatment of a subject in need of a Nanobody, a domain antibody, a single domain antibody or a “dAb” that is directed against a target on a tumor cell, said method comprising orally administering, to said subject a therapeutically effective amount of said Nanobody, domain antibody, single domain antibody or “dAb” and/or of a composition comprising the same.
70. A method for the prevention and/or treatment of at least one disease or disorder that can be prevented and/or treated by administering to a subject suffering from said disease or disorder a Nanobody, a domain antibody, a single domain antibody or a “dAb” that is directed against a target on a tumor cell, said method comprising orally administering to said subject a therapeutically effective amount of said Nanobody, domain antibody, single domain antibody or “dAb” and/or of a composition comprising the same.
71. A method for the prevention and/or treatment of a tumor related disease or disorder, said method comprising orally administering to said subject a therapeutically effective amount of a Nanobody, a domain antibody, a single domain antibody or a “dAb” that is directed against a target on a tumor and/or of a composition comprising the same.
72. Use of a Nanobody, a domain antibody, a single domain antibody or a “dAb” directed against a target on a tumor for the preparation of a composition according to any of embodiments 1 to 50 for the prevention and/or treatment of at least one a tumor related disease or disorder.
73. The composition according to any of embodiments 1 to 50, wherein said Nanobody, domain antibody, single domain antibody or “dAb” is directed against a target on a tumor for the prevention and/or treatment of at least one tumor related disease or disorder.
74. A method for the prevention and/or treatment of a subject in need of a Nanobody, a domain antibody, a single domain antibody or a “dAb” that is directed against a target in gut, said method comprising orally administering, to said subject a therapeutically effective amount of said Nanobody, domain antibody, single domain antibody or “dAb” and/or of a composition comprising the same.
75. A method for the prevention and/or treatment of at least one disease or disorder that can be prevented and/or treated by administering to a subject suffering from said disease or disorder a Nanobody, a domain antibody, a single domain antibody or a “dAb” that is directed against a target in the gut, said method comprising orally administering to said subject a therapeutically effective amount of said Nanobody, domain antibody, single domain antibody or “dAb” and/or of a composition comprising the same.
76. A method for the prevention and/or treatment of a disease or disorder of the gut (such as intestinally located inflammatory diseases such as 1BD or Crohn's disease.
77. A method for the prevention and/or treatment of a subject in need of a Nanobody, a domain antibody, a single domain antibody or a “dAb” that is directed against TNF, said method comprising orally administering, to said subject a therapeutically effective amount of said Nanobody, domain antibody, single domain antibody or “dAb” and/or of a composition comprising the same.
78. A method for the prevention and/or treatment of at least one disease or disorder that can be prevented and/or treated by administering to a subject suffering from said disease or disorder a Nanobody, a domain antibody, a single domain antibody or a “dAb” that is directed against TNF, said method comprising orally administering to said subject a therapeutically effective amount of said Nanobody, domain antibody, single domain antibody or “dAb” and/or of a composition comprising the same.
79. A method for the prevention and/or treatment of a disease or disorder such as an autoimmune disease (such as e.g. rheumatoid arthritis or Inflammatory Bowel Disease), said method comprising orally administering to said subject a therapeutically effective amount of a Nanobody, a domain antibody, a single domain antibody or a “dAb” that is directed against TNF and/or of a composition comprising the same.
80. Use of a Nanobody, a domain antibody, a single domain antibody or a “dAb” directed against TNF for the preparation of a composition according to any of embodiments 1 to 50 for the prevention and/or treatment of at least one disease or disorder such as an autoimmune disease (such as e.g. rheumatoid arthritis or Inflammatory Bowel Disease).
81. The composition according to any of embodiments 1 to 50, wherein said Nanobody, domain antibody, single domain antibody or “dAb” is directed against TNF for the prevention and/or treatment of at least one disease or disorder such as an autoimmune disease (such as e.g. rheumatoid arthritis or Inflammatory Bowel Disease).
82. A method for the prevention and/or treatment of a subject in need of a Nanobody, a domain antibody, a single domain antibody or a “dAb” that is directed against vWF, said method comprising orally administering, to said subject a therapeutically effective amount of said Nanobody, domain antibody, single domain antibody or “dAb” and/or of a composition comprising the same.
83. A method for the prevention and/or treatment of at least one disease or disorder that can be prevented and/or treated by administering to a subject suffering from said disease or disorder a Nanobody, a domain antibody, a single domain antibody or a “dAb” that is directed against vWF, said method comprising orally administering to said subject a therapeutically effective amount of said Nanobody, domain antibody, single domain antibody or “dAb” and/or of a composition comprising the same.
84. A method for the prevention and/or treatment of a disease or disorder related to platelet-mediated aggregation (such as e.g. the formation of a non-occlusive thrombus, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, thrombotic thrombocytopenic purpura (TTP), transient cerebral ischemic attack, unstable or stable angina pectoris, cerebral infarction, HELLP syndrome, carotid endarterectomy, carotid artery stenosis, critical limb ischaemia, cardioembolism, peripheral vascular disease, restenosis and myocardial infarction), said method comprising orally administering to said subject a therapeutically effective amount of a Nanobody, a domain antibody, a single domain antibody or a “dAb” that is directed against vWF and/or of a composition comprising the same.
85. Use of a Nanobody, a domain antibody, a single domain antibody or a “dAb” directed against vWF for the preparation of a composition according to any of embodiments 1 to 50 for the prevention and/or treatment of at least one disease or disorder related to platelet-mediated aggregation (such as e.g. the formation of a non-occlusive thrombus, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, thrombotic thrombocytopenic purpura (TTP), transient cerebral ischemic attack, unstable or stable angina pectoris, cerebral infarction, HELLP syndrome, carotid endarterectomy, carotid artery stenosis, critical limb ischaemia, cardioembolism, peripheral vascular disease, restenosis and myocardial infarction).
86. The composition according to any of embodiments 1 to 50, wherein said Nanobody, domain antibody, single domain antibody or “dAb” is directed against vWF for the prevention and/or treatment of at least one disease or disorder related to platelet-mediated aggregation (such as e.g. the formation of a non-occlusive thrombus, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, thrombotic thrombocytopenic purpura (TTP), transient cerebral ischemic attack, unstable or stable angina pectoris, cerebral infarction, HELLP syndrome, carotid endarterectomy, carotid artery stenosis, critical limb ischaemia, cardioembolism, peripheral vascular disease, restenosis and myocardial infarction).
87. A method for the prevention and/or treatment of a subject in need of a Nanobody, a domain antibody, a single domain antibody or a “dAb” that is directed against IL-6, IL-6R and/or IL-6/IL-6R complex, said method comprising orally administering, to said subject a therapeutically effective amount of said Nanobody, domain antibody, single domain antibody or “dAb” and/or of a composition comprising the same.
88. A method for the prevention and/or treatment of at least one disease or disorder that can be prevented and/or treated by administering to a subject suffering from said disease or disorder a Nanobody, a domain antibody, a single domain antibody or a “dAb” that is directed against IL-6, IL-6R and/or IL-6/IL-6R complex, said method comprising orally administering to said subject a therapeutically effective amount of said Nanobody, domain antibody, single domain antibody or “dAb” and/or of a composition comprising the same.
89. A method for the prevention and/or treatment of a disease or disorder associated with IL-6R, IL-6 and/or with the IL-6/IL-6R complex (such as e.g. sepsis, various forms of cancer such as multiple myeloma disease (MM), renal cell carcinoma (RCC), plasma cell leukaemia, lymphoma, B-lymphoproliferative disorder (BLPD) and prostate cancer, bone resorption (osteoporosis), cachexia, psoriasis, mesangial proliferative glomerulonephritis, Kaposi's sarcoma, AIDS-related lymphoma, inflammatory diseases and disorder such as rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus), said method comprising orally administering to said subject a therapeutically effective amount of a Nanobody, a domain antibody, a single domain antibody or a “dAb” that is directed against IL-6, IL-6R and/or IL-6/IL-6R complex and/or of a composition comprising the same.
90. Use of a Nanobody, a domain antibody, a single domain antibody or a “dAb” directed against IL-6, IL-6R and/or IL-6/IL-6R complex for the preparation of a composition according to any of embodiments 1 to 27 for the prevention and/or treatment of at least one disease or disorder associated with IL-6R, IL-6 and/or with the IL-6/IL-6R complex (such as e.g. sepsis, various forms of cancer such as multiple myeloma disease (MM), renal cell carcinoma (RCC), plasma cell leukaemia, lymphoma. B-lymphoproliferative disorder (BLPD) and prostate cancer, bone resorption (osteoporosis), cachexia, psoriasis, mesangial proliferative glomerulonephritis, Kaposi's sarcoma, AIDS-related lymphoma, inflammatory diseases and disorder such as rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus).
91. The composition according to any of embodiments 1 to 50, wherein said Nanobody, domain antibody, single domain antibody or “dAb” is directed against IL-6, IL-6R and/or IL-6/IL-6R complex for the prevention and/or treatment of at least one disease or disorder associated with IL-6R, IL-6 and/or with the IL-6/IL-6R complex (such as e.g. sepsis, various forms of cancer such as multiple myeloma disease (MM), renal cell carcinoma (RCC), plasma cell leukaemia, lymphoma, B-lymphoproliferative disorder (BLPD) and prostate cancer, bone resorption (osteoporosis), cachexia, psoriasis, mesangial proliferative glomerulonephritis, Kaposi's sarcoma, AIDS-related lymphoma, inflammatory diseases and disorder such as rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma (in particular allergic asthma) and autoimmune insulin-dependent diabetes mellitus).
92. A method for the prevention and/or treatment of an acute disorder or disease, said method comprising orally administering to said subject a therapeutically effective amount of a Nanobody, a domain antibody, a single domain antibody or a “dAb” that is capable of alleviating the symptoms of or curing said disorder or disease.
93. A method for selecting Nanobodies, domain antibodies, single domain antibodies or “dAbs” directed against an epithelial trans-membrane protein, wherein said Nanobodies, domain antibodies, single domain antibodies or “dAbs” cross the membrane upon binding to said epithelial trans-membrane protein, said method comprising the step of panning epithelial trans-membrane protein-displaying membranes with a phage library (naïve or immune) of Nanobodies, domain antibodies, single domain antibodies or “dAbs” and selecting for membrane crossing Nanobodies, domain antibodies, single domain antibodies or “dAbs” by recovering the transported phage from the membrane.
94. Diagnostic method or drug monitoring method comprising the step of orally administering to a subject a Nanobody, a domain antibody, a single domain antibody or a “dAb” or a composition comprising the same and detecting said Nanobody, domain antibody, single domain antibody or “dAb”.
95. Method according to embodiment 94, wherein said Nanobody, domain antibody, single domain antibody or “dAb” is detected in situ.

1. A pharmaceutical composition for oral administration comprising a therapeutically effective amount of a polypeptide comprising one or more single variable domain(s) and a pharmaceutically acceptable enteric coating.

2. The composition of claim 1, wherein the polypeptide comprises 2 single variable domains.

3. The composition of claim 2, wherein the single variable domains are 2 identical Nanobodies.

4. The composition of claim 1, wherein the polypeptide has agonistic properties.

5. The composition of claim 1, wherein the polypeptide also comprises a single variable domain against FcRn, pIgR or Vit B12 receptor.

6. The composition of claim 5 wherein the binding of the single variable domain against FcRn, pIgR or Vit B12 receptor is pH dependent.

7. The composition of claim 1 additionally comprising a permeability enhancer.

8. The composition of claim 1 additionally comprising a protease inhibitor.


SEQUENCE LISTING (50 sequences)

SEQ ID NO: 1 - protein, 119 aa, Artificial
  polypeptide comprising at least one single variable domain
EVQLVESGGGLVQPGGSLRLSCAASGFTFSDHAMSWVRQAPGKGLEWVSAINTAGIITNYADSVKGRFTMSRDNAKNTLYLQMNSLKPEDTAKYYCARNRDINNSRPPESQGTQVTVSS

SEQ ID NO: 2 - protein, 119 aa, Artificial
  polypeptide comprising at least one single variable domain
EVQLVESGGGLVQPGGSLRLSCAASGFTFSDYAMSWVRQAPGKGLEWVSAINTAGIITNYADSVKGRFTMSRDNAKNTLYLQMNSLKPEDTAKYYCARNRDINNSRPPESQGTQVTVSS

SEQ ID NO: 3 - protein, 119 aa, Artificial
  polypeptide comprising at least one single variable domain
EVQLVESGGGLVQPGGSLRLSCAASGFTFSDYAMSWVRQAPGKGLEWVSAINTAGILTNYADSVKGRFTMSRDNAKNTLYLQMNSLKPEDTGKYYCARNRDINNSRPPESQGTQVTVSS

SEQ ID NO: 4 - protein, 125 aa, Artificial
  polypeptide comprising at least one single variable domain
EVQLVESGGGSVQPGGSLRLSCLASGRTFSTYFMGWFRQAPGKEREFVTAISASGGTTYYADSVKGRFTISRDNAKNTVYLQMNSLKPEDTAVYYCAANNRYYSDYMKLWPYDYWGQGTQVTVSS

SEQ ID NO: 5 - protein, 119 aa, Artificial
  polypeptide comprising at least one single variable domain
EVQLVESGGGLVQPGGSLRLSCVASAYIFNINHMGWYRQAPGKRREVVAAVSSAGNTYYADSVKGRFTISRDDAKNTVYLQMNNLKPEDTAVYYCNRLAPTGGTWPDYWGQGTQVTVSS

SEQ ID NO: 6 - protein, 125 aa, Artificial
  polypeptide comprising at least one single variable domain
EVQLVKSGGGLVQAGGSLRLSCAASGRSFGYYAMAWFRQAPGKEREFVAAFTWNGVSTTTYYADSVKGRFTISRDNAKNTGYLQMNSVRPEDTAVYYCAARSGFYSRQKSEYPYWGQGTQVTVSS

SEQ ID NO: 7 - protein, 121 aa, Artificial
  polypeptide comprising at least one single variable domain
EVQLVESGGGLVQSGGSLRLSCAASGRAFNYYAMGWFRQAPGKERELVAVINWSGGTTSYADSVKGRFTISRSNAKNTVYLQMSSLKPEDTAVYYCAADSIYRTSKDYNYWGQGTQVTVSS

SEQ ID NO: 8 - protein, 127 aa, Artificial
  polypeptide comprising at least one single variable domain
EVQLVESGGGLVQAGGSLRLSCAASGRTFSNYVMGWFRQAPGKEREFVAAISWSGVTTYHYSADSVKGRFTTSRDNDRNTAHLQMNSLKPEDTAVYYCAARGRTGSDPRKGDDYDYWGQGTQVTVSS

SEQ ID NO: 9 - protein, 129 aa, Artificial
  polypeptide comprising at least one single variable domain
EVQLVESGGGLVQPGGSLRLSCAASGFTLDYYAIGWFRRAPGKEREGVSCTSSSDGSTYYADSVKGRFTISRDNAKNTVYLQMNSLKPEDTAVYYCATTFGDACTVVAGIPDQYDFGSWGQGTQVTVSS

SEQ ID NO: 10 - protein, 132 aa, Artificial
  polypeptide comprising at least one single variable domain
EVQLVESGGGLVQPGGSLRLSCAASGFTLDYYAIGWFRQAPGKEREGVSCISSSDMSDGITYYADSVKGRFTSSRDNVKNTVYLQMNSLKPEDTAVYYCATTFGDACTVVAGIPDQADFDSWGQGTQVTVSS

SEQ ID NO: 11 - protein, 129 aa, Artificial
  polypeptide comprising at least one single variable domain
EVQLVESGGGLVQPGGSLRLSCAASGFTLDYYDIGWFRQAPGKEREGVSCTSSSDGSTYYADSVKGRFTISRDNAKNTVYLQMNSLKPEDTAVYYCATTFGDACTVVAGIPDQYDFGSWGQGTQVTVSS

SEQ ID NO: 12 - protein, 129 aa, Artificial
  polypeptide comprising at least one single variable domain
EVQLVESGGGLVQAGGSLRLSCEDSGRTFGDYIMGWFRQAPGKERDFVAAISWTGDSTYYKYYSDSAKGRFTASRDNAKNTAYLQMNGLKPEDTAVYYCAARTFRIDYDPRTASTYNYWGQGIQVTVSS

SEQ ID NO: 13 - protein, 127 aa, Artificial
  polypeptide comprising at least one single variable domain
EVQLVKSGGGLVQAGDSLRLSCAASGRTSSSVTMGWFRQAPGKERDFVAAISWSGGSTYYADSVKGRFTISRDNAKNTVYLQMNSLKPEDTAVYYCAAVVPSDPIIYYTDYVDYDYWGQGTQVTVSS

SEQ ID NO: 14 - protein, 124 aa, Artificial
  polypeptide comprising at least one single variable domain
EVQLVESGGGLVQAGGSLRLSCAASGFTFDDYAIGWFRQAPGKEREGVSCISSRDGITYYADSVKGRFTISSDNAKNTVYLQMNSLKPEDTAVYYCAADLVGSFPCPVAAYDYWGQGTQVTVSS

SEQ ID NO: 15 - protein, 124 aa, Artificial
  polypeptide comprising at least one single variable domain
EVQLVESGGGLVQAGGSLRLSCAASGFTFDDYAIGWFRQAPGKEREGVSCISSRDGTTYYADSVKGRFTISSDNAKNTVYLQMNSLKPEDTAVYYCAADLVGSFPCPVAAYDYWGQGTQVTVSS

SEQ ID NO: 16 - protein, 124 aa, Artificial
  polypeptide comprising at least one single variable domain
EVQLVESGGGLVQAGGSLRLSCAASGFTFDDYAIGWFRQAPGKEREGVSCINNSDGSTYYADSVKGRFTISSDNAKNTVYLQMNSLKPEDTAVYYCAADFVGGSYCLFPTYNYWGQGTQVTVSS

SEQ ID NO: 17 - protein, 123 aa, Artificial
  polypeptide comprising at least one single variable domain
EVQLVESGGGLVQAGDSLRLSCAASGRTFSTYAMAWFRQAPGKDREFVAAISWSSDMTYYLDSVKGRFTISRDNAKNTVFLQMNSLKPEDTAVYYCASGAYYAGSSTSPYNYWGQGTQVTVSS

SEQ ID NO: 18 - protein, 129 aa, Artificial
  polypeptide comprising at least one single variable domain
EVQLVESGGGLVQAGGSLRLSCAASGRTFTIYTMGWFRQAPGKAREFISALRWSGGSTYTFYADSVKGRFTISRDNAKNTLYLQMNSLKSEDTAIYYCGMVDPRAPYMRPDSTDSYAYWGQGTQVTVSS

SEQ ID NO: 19 - protein, 128 aa, Artificial
  polypeptide comprising at least one single variable domain
EVQLVESGGGQVQAGGSLRLSCVASERTFSYYDLAARAWFRQAPGKERELVSASTWNGGYTYYVDSVKGRFTVSTDDAGDTMYLQMNSLEPEDTAVYYCAARRAYSSDLHDYRTFDYWGQGTQVTVSS

SEQ ID NO: 20 - protein, 124 aa, Artificial
  polypeptide comprising at least one single variable domain
EVQLVESGGGLVQAGGSLRLSCAASGFTFDDYAIGWFRQAPGKEREWVSCISRSDGSTYYADSVKGRFTISSDNAKNTVYLQMNSLKPEDTAVYHCAADAIGSFPCPAGVYDYWGQGTQVTVSS

SEQ ID NO: 21 - protein, 131 aa, Artificial
  polypeptide comprising at least one single variable domain
EVQLVESGGGLAQPGGSLRLSCAASGFAFSSYWMYWVRQAPGKGLEWVSAISTGGGGTYYADSVKGRFTISRDNAKNTLYLQMNSLKPEDTARYYCARDEAPTFDYSGNYAYTGSDPNDYWGQGTQVTVSS

SEQ ID NO: 22 - protein, 130 aa, Artificial
  polypeptide comprising at least one single variable domain
EVQLVESGGGLVQPGGSLRLSCAASGFAFSRYGMYWVRQAPGKGLEWVSTINSGGDYIIYADSVKGRFTISRDNAKNTLYLQMNSLKPEDTAVYYCAEGYRGSEWPPPAFTLQRADFASWGQGTQVTVSS

SEQ ID NO: 23 - protein, 126 aa, Artificial
  polypeptide comprising at least one single variable domain
EVQLVESGGGLVQAGGSLRLSCVASGFILREYNMGWFRQAPGKEREIVAAIAWTGTNSYYVDSVKGRFTISRDDTKNTVYLQMNSLNPEDTGVYHCAAEGYVSNFPRSSADEYDYWGQGTQVTVSS

SEQ ID NO: 24 - protein, 117 aa, Artificial
  polypeptide comprising at least one single variable domain
EVQLVESGGGLVQPGGSLRLSCAASGFTFSSYAMTWVRQGLGKGLEWVSIISGNGGSTSYADSVKGRFTISRDNAKNTLYLQMNSLKPEDTAVYYCAKWDGLGTLPGSQGTQVTVSS

SEQ ID NO: 25 - protein, 124 aa, Artificial
  polypeptide comprising at least one single variable domain
EVQLVESGGGLVQAGGSLRLSCAASGSILSTNDMGWYRQAPGNQRELVARISRGSSTIYTESVKGRFTISRDNAKNTVYLQMNSLKPEDTAVYYCNADMLPSDLSHGYYYRDYWGQGTQVTVSS

SEQ ID NO: 26 - protein, 124 aa, Artificial
  polypeptide comprising at least one single variable domain
EVQLVESGGGLVQAGGSLRLSCAASGFTFDDYAIGWFRQAPGKEREGVSCISSRDGMTYYADSVKGRFTISSDNARNTVYLQMNSLKPEDTAVYYCAADLVGSFPCPVAAYDYWGQGTQVTVSS

SEQ ID NO: 27 - protein, 119 aa, Artificial
  polypeptide comprising at least one single variable domain
EVQLVESGGGLVQAGDSLTLSCVASGRTFSAYGMGWFRQAPGKEREFVASINWGGGNTYYANSVKDRFAISKDHAKNTVYLQMNSLKPEDTALYYCAAVSSNTEIFDTWGQGIQVTVSS

SEQ ID NO: 28 - protein, 114 aa, Artificial
  polypeptide comprising at least one single variable domain
EVQLVESGGGLVQPEGSLRLSCRASGSIFSINTMGWYRQPPGKEREFVARIYPGITHYADSVKGRFTISRDNAKNTVYLQMNSLKPEDTAVYYCFYYYDDRNYWGEGTLVTVSS

SEQ ID NO: 29 - protein, 126 aa, Artificial
  polypeptide comprising at least one single variable domain
EVQLVESGGGLVQAGGSLRLSCAASGLSFSSYAMGWFRQAPGKEREFVAAIRRREGNSYYADSVKGRFTISRDSAKNTVYLQMNSLKPEDTALYSCAATAPHYSGSFAYAGGYDYWGQGTQVTVSS

SEQ ID NO: 30 - protein, 123 aa, Artificial
  polypeptide comprising at least one single variable domain
EVQLVESGGGLVQPGGSLRLSCAASPFTLDYYAIGWFRQAPGKEREGVSCSTSHGKTYHADSVKGRFTISRDNAKNTVYLQMNSLKPEDTAVYYCAAGACMGGSGYEADFGSWGQGTQVTVSS

SEQ ID NO: 31 - protein, 123 aa, Artificial
  polypeptide comprising at least one single variable domain
EVQLVESGGGLVQAGGSLRLSCEASGFTLDYSGVGWFRQAPGKERELVSCISRGGDRAGYANSVKGRFTMSRDNAKNILYLQMNSLKPEDTAVYYCAATHSGSGCYDGAIDYWGKGTLVTVSS

SEQ ID NO: 32 - protein, 123 aa, Artificial
  polypeptide comprising at least one single variable domain
EVQLVESGGGLVQPGGSLRLSCVAAGFTLDYYAIGWFRQAPGKEREGVSCITSDGRTYYADSVKGRFTISRDMAKKMVYLQMNSLKPEDTAVYYCAAGACMGGSGYEADFGSWGQGTQVTVSS

SEQ ID NO: 33 - protein, 126 aa, Artificial
  polypeptide comprising at least one single variable domain
EVQLVESGGGLVQAGDSLRLSCAASGRTYSRNAMAWFRQAPGKEREFVAGIDWSSENTRYIDSVKGRFTISRDNAKSTMYLQMNSLKPEDTAVYYCAAGTSWGALASRLEAAYSSWGQGTQVTVSS

SEQ ID NO: 34 - protein, 126 aa, Artificial
  polypeptide comprising at least one single variable domain
KVQLVESGGGLVQVGGSLRLSCVASGRTYGGNAMAWFRQAPGKEREFVAGIDWSSENTRYTDSVKGRFTISRDNAKNTMYLQMNSLKPEDTAVYYCAAGTSWGALASRLENAYSAWGQGTQVTVSS

SEQ ID NO: 35 - protein, 126 aa, Artificial
  polypeptide comprising at least one single variable domain
EVQLVESGGGLVQAGGSLRLSCAASGGTYSGNAMAWFRQAPGKEREFVAGIDWSSENTRYIDSVKGRFTISRDNAKNTMYLQMNSLKPEDTAVYYCAAGTSWGALASRLEAAYSSWGQGTQVTVSS

SEQ ID NO: 36 - protein, 123 aa, Artificial
  polypeptide comprising at least one single variable domain
EVQLVESGGGLVQAGDSLRLSCAASGRTFSSYAMGWFRQAPGTERQFVARITGKGDSTDYADSVRGRFTISRDNAKNTVYLQMNSLKPEDTAVYYCAADAFNSLLQAGRAEYWGQGTQVTVSS

SEQ ID NO: 37 - protein, 122 aa, Artificial
  polypeptide comprising at least one single variable domain
EVQLVESGGGLVQAGGSLRLSCAASGRTFSSYAMAWFRQAPGEEREFVATISVSGGYTYYADSVKGRFTISRDNAKNTVYLQMNTLKPEDTAVYYCAAGDSSSWLEHMYDYWGQGTQVTVSS

SEQ ID NO: 38 - protein, 124 aa, Artificial
  polypeptide comprising at least one single variable domain
EVQLVESGGGLVQAGDSLRLSCAASGRPFMSYVMGWFRRAPGKEREFVGGINWGSGNTWYTDSVLGRFTISRDNAKNTVYLQMNSLKPEDTAVYYCATAAGVGYYRYERQYDYWGQGTQVTVSS

SEQ ID NO: 39 - protein, 124 aa, Artificial
  polypeptide comprising at least one single variable domain
EVQLVESGGGLVQAGDSLRLSCAASGRPFSAYVMGWFRRAPGKEREFVGGINWNSANTWYTDSVLGRFTISKDNAKNTVYLQMNSLKPEDTAVYYCAAAGGVGYYRYERQYDYWGQGTQVTVSS

SEQ ID NO: 40 - protein, 124 aa, Artificial
  polypeptide comprising at least one single variable domain
EVQLVESGGGLVQAGDSLRLSCAASGRTYTPYVMGWFRRAPGKEREFVGAVSWSGTNTWYTDSVKGRFTISRDNAKNTVYLQMNSLKPEDTAVYYCAAGDGVGIYRYEHQYDYWGQGTQVTVSS

SEQ ID NO: 41 - protein, 124 aa, Artificial
  polypeptide comprising at least one single variable domain
EVQLVESGGGLVQAGDSLRLSCTASERPFSTYVMGWFRRAPGKEREFVGGITWSGINAWYTDSVLGRFTISSDNAKNTVYLQMNSLKPEDTAVYYCAAASGVGRYRYELQYDYWGQGTQVTVSS

SEQ ID NO: 42 - protein, 123 aa, Artificial
  polypeptide comprising at least one single variable domain
EVQLVESGGGLVQAGGSLRLSCAASGLSFSRYAMGWFRQAPGKQREYVAVISSSDTTYYTNSAKGRFTISRDNAENTVYLQMNSLKPEDTAVYFCAADSFVTALQTLTQINYWGQGTQVTVSS

SEQ ID NO: 43 - protein, 119 aa, Artificial
  polypeptide comprising at least one single variable domain
EVQLVKSGGGLVQPGGSLRLSCAASGFTFSHYQMSWVRQAPGKDVEWVSSISMLGGGTTYADSVKGRFTISRDNAKSTLVLQMNNLKVEDTAVYYCARGFSGNYYRADLGQGTQVTVSS

SEQ ID NO: 44 - protein, 124 aa, Artificial
  polypeptide comprising at least one single variable domain
EVQLVESGGGLVQAGDSLRLSCAASGRTFSPYVMGWFRRAPGKEREFVGGINWSGSNTWYTDSVKGRFTISRDNVKNMVYLQMNSLKPEDTAVYYCAAGSGVGMYRYERQYDYWGQGTQVTVSS

SEQ ID NO: 45 - protein, 123 aa, Artificial
  polypeptide comprising at least one single variable domain
EVQLVESGGGLVQAGGSLRLSCAASGLSFSKYAMGWFRQAPGKQREYVAVISSSDTTYYTNSAKGRFTISRDNAENTVYLQMNSLKPEDTAVYFCAADSYVTALQTLTQISYWGQGTQVTVSS

SEQ ID NO: 46 - protein, 124 aa, Artificial
  polypeptide comprising at least one single variable domain
EVQLVESGGGLVQAGDSLRLSCAASGRPFSSYVMGWFRRAPGKEREFVGGINWNSGNTWYSDSVLGRFTISRDNAKNTVDLQMNSLKPEDTAVYYCATASGVGYYRYERQYDYWGQGTQVTVSS

SEQ ID NO: 47 - protein, 124 aa, Artificial
  polypeptide comprising at least one single variable domain
EVQLVESGGGLVQAGDSLRLSCTASERPFMSYVMGWFRRAPGKDREFVGAITWSGINTWYSDSVLGRFTISRDNAKNTVYLQMNSLKPEDTAVYYCAVADGVGLYRYERQYDYWGQGTQVTVSS

SEQ ID NO: 48 - protein, 251 aa, Artificial
  polypeptide comprising at least one single variable domain
EVQLVESGGGLVQPGGSLRLSCAASGFTFSDYDIGWFRQAPGKGREGVSGISSSDGNTYYADSVKGRFTISRDNAKNTLYLQMNSLRPEDTAVYYCAAEPPDSSWYLDGSPEFFKYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 49 - protein, 115 aa, Artificial [sequence omitted]

SEQ ID NO: 50 - protein, 260 aa, Artificial
  polypeptide comprising at least one single variable domain
EVQLVESGGGLVQPGGSLRLSCAASGFTFSDYDIGWFRQAPGKGREGVSGISSSDGNTYYADSVKGRFTISRDNAKNTLYLQMNSLRPEDTAVYYCAAEPPDSSWYLDGSPEFFKYWGQGTLVTVSSGGGGSGGGSEVQLVESGGGLVQAGDSLRLSCTASERPFMSYVMGWFRRAPGKDREFVGAITWSGINTWYSDSVLGRFTISRDNAKNTVYLQMNSLKPEDTAVYYCAVADGVGLYRYERQYDYWGQGTQVTVSS